SEQUENCE LISTING (12 sequences)

SEQ ID NO: 1 - protein, 51 aa, synthetic construct
  REGION 1..51: note = Description of Artificial Sequence: Synthetic polypeptide
  REGION 1..20: note = MISC_FEATURE - This region may encompass 0-20 residues
  REGION 32..51: note = MISC_FEATURE - This region may encompass 0-20 residues
  REGION 1..51: note = See specification as filed for detailed description of substitutions and preferred embodiments
GGGGGGGGGGGGGGGGGGGGRGRDDRRQRRRGGGGGGGGGGGGGGGGGGGG

SEQ ID NO: 2 - protein, 51 aa, synthetic construct
  REGION 1..51: note = Description of Artificial Sequence: Synthetic polypeptide
  REGION 1..20: note = MISC_FEATURE - This region may encompass 0-20 residues
  REGION 32..51: note = MISC_FEATURE - This region may encompass 0-20 residues
  REGION 1..51: note = See specification as filed for detailed description of substitutions and preferred embodiments
GGGGGGGGGGGGGGGGGGGGYGRKKRRQRRRGGGGGGGGGGGGGGGGGGGG

SEQ ID NO: 3 - protein, 49 aa, synthetic construct
  REGION 1..49: note = Description of Artificial Sequence: Synthetic polypeptide
  REGION 1..20: note = MISC_FEATURE - This region may encompass 0-20 residues
  REGION 30..49: note = MISC_FEATURE - This region may encompass 0-20 residues
  REGION 1..49: note = See specification as filed for detailed description of substitutions and preferred embodiments
GGGGGGGGGGGGGGGGGGGGRKKRRQRRRGGGGGGGGGGGGGGGGGGGG

SEQ ID NO: 4 - protein, 35 aa, synthetic construct
  REGION 1..35: note = Description of Artificial Sequence: Synthetic polypeptide
RKKRRQRRRGKKKKKKKKKKKKKKKGRKKRRQRRR

SEQ ID NO: 5 - protein, 45 aa, synthetic construct
  REGION 1..45: note = Description of Artificial Sequence: Synthetic polypeptide
  REGION 1..20: note = MISC_FEATURE - This region may encompass 0-20 residues
  REGION 21..45: note = MISC_FEATURE - This region may encompass 5, 7, 9, 11, 13, 15, 17, 19 21, 23 or 25 residues
  REGION 1..45: note = See specification as filed for detailed description of substitutions and preferred embodiments
GGGGGGGGGGGGGGGGGGGGRRRRRRRRRRRRRRRRRRRRRRRRR

SEQ ID NO: 6 - protein, 19 aa, Drosophila sp.
SGRQIKIWFQNRRMKWKKC

SEQ ID NO: 7 - protein, 39 aa, synthetic construct
  REGION 1..39: note = Description of Artificial Sequence: Synthetic polypeptide
YGRKKRRQRRRGKKKKKKKKKKKKKKKGYGRKKRRQRRR

SEQ ID NO: 8 - protein, 39 aa, synthetic construct
  REGION 1..39: note = Description of Artificial Sequence: Synthetic polypeptide
RGRDDRRQRRRGKKKKKKKKKKKKKKKGRGRDDRRQRRR

SEQ ID NO: 9 - protein, 20 aa, synthetic construct
  REGION 1..20: note = Description of Artificial Sequence: Synthetic peptide
  REGION 1..20: note = MISC_FEATURE - This sequence may encompass 10-20 residues
  REGION 1..20: note = See specification as filed for detailed description of substitutions and preferred embodiments
KKKKKKKKKKKKKKKKKKKK

SEQ ID NO: 10 - protein, 10 aa, synthetic construct
  REGION 1..10: note = Description of Artificial Sequence: Synthetic peptide
GGGRRRRRRR

SEQ ID NO: 11 - protein, 35 aa, synthetic construct
  REGION 1..35: note = Description of Artificial Sequence: Synthetic polypeptide
RKKRRQRRRQKKKKKKKKKKKKKKKQRKKRRQRRR

SEQ ID NO: 12 - protein, 7 aa, synthetic construct
  REGION 1..7: note = Description of Artificial Sequence: Synthetic peptide
KLAKLAK

(19) United States
(12) Patent Application Publication
(10) Pub. No.: US 2025/0064905 A1
(43) Pub. Date: Feb. 27, 2025

(54) INJECTABLE BOTULINUM TOXIN FORMULATIONS AND METHODS OF USE THEREOF HAVING HIGH RESPONSE RATE AND LONG EFFECT DURATION

(71) Applicant: REVANCE THERAPEUTICS, INC., Newark, CA (US)

(72) Inventor: Roman Rubio, Redwood City, CA (US)

(21) Appl. No.: 18/595,365

(22) Filed: Mar. 4, 2024

Related U.S. Application Data
(63) Continuation of application No. 16/770,033, filed on Jun. 4, 2020, now abandoned.
(60) Provisional application No. 62/594,529, filed on Dec. 4, 2017.
(60) Provisional application No. 62/774,850, filed on Dec. 3, 2018.

(51) Int. Cl. A61K 38/48; A61K 8/60; A61K 8/66; A61K 9/00; A61K 47/26; A61K 47/64; A61P 17/00; A61Q 19/08

(52) U.S. Cl. CPC A61K 38/4893; A61K 8/60; A61K 8/66; A61K 47/26; A61K 47/645; A61P 17/00; A61Q 19/08; A61K 9/0019; C12Y 304/24069

(57) ABSTRACT

This invention provides injectable compositions comprising botulinum toxin that may be administered to a subject for various therapeutic, aesthetic and/or cosmetic purposes. The injectable compositions embraced by the invention exhibit one or more advantages over conventional botulinum toxin formulations, including reduced antigenicity, a reduced tendency to undergo unwanted localized diffusion following injection, increased duration of clinical efficacy or enhanced potency, higher responder rates, faster onset of clinical efficacy, and/or improved stability. According to the invention, single treatment of the compositions by injection affords significant clinical responses and at least a 26-week duration of effect in a subject undergoing treatment, as provided by the described treatment methods, as well as still higher responder rates and/or longer duration of effect following subsequent treatments.

### CROSS REFERENCE TO RELATED APPLICATIONS

This application is a continuation of U.S. patent application Ser. No. 16/770,033 filed Jun. 4, 2020 which claims benefit of priority to U.S. Provisional Patent Application No. 62/594,529, entitled “Injectable Botulinum Toxin Formulations and Methods of Use Thereof Having High Response Rate and Long Duration of Effect,” to Rubio, filed on Dec. 4, 2017, and claims benefit of priority to U.S. Provisional Patent Application 62/774,850, entitled “Injectable Botulinum Toxin Formulations and Methods of Use Thereof Having High Response Rate and Long Duration of Effect,” to Rubio, filed on Dec. 3, 2018, both of which are incorporated herein in their entireties.

### SEQUENCE LISTING

The contents of the electronic sequence listing submitted herewith as file “13720.0029U4 2024_06_07”; Size: 22,461 bytes; and Date of Creation: Jun. 7, 2024, is herein incorporated by reference in its entirety.

### FIELD OF THE INVENTION

This invention relates to injectable compositions comprising botulinum toxin that may be administered to a subject for various therapeutic, aesthetic, and/or cosmetic purposes. The injectable compositions and methods in which these compositions are used provide advantageous treatments which result in high responder rates and long duration of effect, for example, a duration of effect for 26 to 40 weeks, as well as still higher responder rates and/or longer duration of effect following subsequent treatments.

### BACKGROUND OF THE INVENTION

Skin protects the body's organs from external environmental threats and acts as a thermostat to maintain body temperature. It consists of several different layers, each with specialized functions. The major layers include the epidermis, the dermis and the hypodermis. The epidermis is a stratifying layer of epithelial cells that overlies the dermis, which consists of connective tissue. Both the epidermis and the dermis are further supported by the hypodermis, an internal layer of adipose tissue.

The epidermis, the topmost layer of skin, is only 0.1 to 1.5 millimeters thick (Inlander, Skin, New York, N.Y.: People's Medical Society, 1-7 (1998)). It consists of keratinocytes and is divided into several layers based on their state of differentiation. The epidermis can be further classified into the stratum corneum and the viable epidermis, which consists of the granular melphigian and basal cells. The stratum corneum is hygroscopic and requires at least 10% moisture by weight to maintain its flexibility and softness. The hygroscopicity is attributable in part to the water-holding capacity of keratin. When the horny layer loses its softness and flexibility it becomes rough and brittle, resulting in dry skin.

The dermis, which lies just beneath the epidermis, is 1.5 to 4 millimeters thick. It is the thickest of the three layers of the skin. Most of the skin's structures, including sweat and oil glands (which secrete substances through openings in the skin called pores, or comedos), hair follicles, nerve endings, and blood and lymph vessels are found in the dermis (Inlander, Skin, New York, N.Y.: People's Medical Society, 1-7 (1998)). However, the main components of the dermis are collagen and elastin.

The hypodermis is the deepest layer of the skin. It acts both as an insulator for body heat conservation and as a shock absorber for organ protection (Inlander, Skin, New York, N.Y.: People's Medical Society, 1-7 (1998)). In addition, the hypodermis also stores fat for energy reserves. The pH of skin is normally between 5 and 6. This acidity is due to the presence of amphoteric amino acids, lactic acid, and fatty acids from the secretions of the sebaceous glands. The term “acid mantle” refers to the presence of the water-soluble substances on most regions of the skin. The buffering capacity of the skin is due in part to these secretions stored in the skin's horny layer.

Wrinkles, one of the telltale signs of aging, can be caused by biochemical, histological, and physiologic changes that accumulate from environmental damage to the skin. (Benedetto, International Journal of Dermatology, 38:641-655 (1999)). In addition, there are other secondary factors that can cause characteristic folds, furrows, and creases of facial wrinkles (Stegman et al., The Skin of the Aging Face Cosmetic Dermatological Surgery, 2^{nd} ed., St. Louis, Mo.: Mosby Year Book: 5-15 (1990)). These secondary factors include the constant pull of gravity, frequent and constant positional pressure on the skin (e.g., during sleep), and repeated facial movements caused by the contraction of facial muscles (Stegman et al., The Skin of the Aging Face Cosmetic Dermatological Surgery, 2^{nd} ed., St. Louis, Mo.: Mosby Year Book: 5-15 (1990)).

Different techniques have been utilized in order to potentially mollify some of the signs of aging. These techniques range from facial moisturizers containing alpha hydroxy acids and retinal to surgical procedures and injections of neurotoxins. For example, in 1986, Jean and Alastair Carruthers, a husband and wife team consisting of an ocuplastic surgeon and a dermatologist, developed a method of using the type A form of botulinum toxin for treatment of movement-associated wrinkles in the glabella area (Schantz and Scott, In Lewis G. E. (Ed) Biomedical Aspects of Botulinum, New York: Academic Press, 143-150 (1981)). The Carruthers' use of the type A form of botulinum toxin for the treatment of wrinkles led to the seminal publication of this approach in 1992 (Schantz and Scott, In Lewis G. E. (Ed) Biomedical Aspects of Botulinum, New York: Academic Press, 143-150 (1981)). By 1994, the same team reported experiences with other movement-associated wrinkles on the face (Scott, Ophthalmol, 87:1044-1049 (1980)). This in turn led to the birth of the era of cosmetic treatment using the type A form of botulinum toxin.

The type A form of botulinum toxin is reported to be the most lethal natural biological agent known to man. Spores of Clostridium botulinum are found in soil and can grow in improperly sterilized and sealed food containers. Botulism, which may be fatal, can be caused by the ingestion of the bacteria. Botulinum toxin acts to produce paralysis of muscles, preventing synaptic transmission by inhibiting the release of acetylcholine across the neuromuscular junction, and is thought to act in other ways as well. Its action essentially blocks signals that normally would cause muscle spasms or contractions, resulting in paralysis. During the last decade, botulinum toxin's muscle paralyzing activity has been harnessed to achieve a variety of therapeutic effects. Controlled administration of botulinum toxin has been used to provide muscle paralysis to treat a variety of medical conditions, for example, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temporomandibular joint disorder, and various types of muscle cramping and spasms. More recently, the muscle-paralyzing effects of botulinum toxin have been applied to therapeutic and cosmetic facial applications such as treatment of wrinkles, frown lines, and other results of spasms or contractions of facial muscles.

In addition to the type A form of botulinum toxin, there are seven other serologically distinct forms of botulinum toxin that are also produced by the gram-positive bacteria C. botulinum. Of these eight serologically distinct types of botulinum toxin, the seven that can cause paralysis have been designated botulinum toxin serotypes A, B, C, D, E, F and G. Each of these is distinguished by neutralization with type-specific antibodies. The different serotypes of botulinum toxin vary in the effect and in the severity and duration of the paralysis they evoke in different animal species. For example, in rats, it has been determined that botulinum toxin type A is 500 times more potent than botulinum toxin type B, as measured by the rate of paralysis. Additionally, botulinum toxin type B has been determined to be non-toxic in primates at a dose of 480 U/kg, about 12 times the primate LDso for type A

As released by C. botulinum bacteria, botulinum toxin is a component of a toxin complex containing the approximately 150 kD botulinum toxin protein molecule along with associated non-toxin proteins. These endogenous non-toxin proteins are believed to include a family of hemagglutinin proteins, as well as non-hemagglutinin protein. The non-toxin proteins have been reported to stabilize the botulinum toxin molecule in the toxin complex and protect it against denaturation by digestive acids when the toxin complex is ingested. Thus, the non-toxin proteins of the toxin complex protect the activity of the botulinum toxin and thereby enhance systemic penetration when the toxin complex is administered via the gastrointestinal tract. Additionally, it is believed that some of the non-toxin proteins specifically stabilize the botulinum toxin molecule in blood.

The presence of non-toxin proteins in the toxin complexes typically causes the toxin complexes to have molecular weights that are greater than that of the bare botulinum toxin molecule. The molecular weight botulinum toxin protein itself is about 150 kD, though the different serotype complexes vary in size. For example, C. botulinum bacteria can produce botulinum type A toxin complexes that have molecular weights of about 900 kD, 500 kD or 300 kD. Botulinum toxin types B and C are produced as complexes having a molecular weight of about 700 kD or about 500 kD. Botulinum toxin type D is produced as complexes having molecular weights of about 300 kD or 500 kD. Botulinum toxin types E and Fare only produced as complexes having a molecular weight of about 300 kD.

To provide additional stability to botulinum toxin, the toxin complexes are conventionally stabilized by combining the complexes with albumin during manufacturing. For example, BOTOX® (Allergan, Inc., Irvine, CA) is a botulinum toxin-containing formulation that contains 100 U of type A botulinum toxin with accessory proteins, 0.5 milligrams of human albumin, and 0.9 milligrams of sodium chloride.

Due to the molecule size and molecular structure of botulinum toxin, it does not on its own cross the stratum corneum of the skin and the multiple layers of the underlying skin architecture. Accordingly, the botulinum toxin typically is administered to patients by injection of compositions containing botulinum toxin complex and albumin. However, there are several problems associated with this approach. Not only are the injections painful, but typically large subdermal wells of toxin are locally generated around the injection sites, in order to achieve the desired therapeutic or cosmetic effect. The botulinum toxin may migrate from these subdermal wells to cause unwanted paralysis in surrounding areas of the body. This problem is exacerbated when the area to be treated is large and many injections of toxin are required to treat the area. Moreover, because the injected toxin complexes contain non-toxin proteins and albumin that stabilize the botulinum toxin and increase the molecular weight of the toxin complex, the toxin complexes have a long half-life in the body and may cause an undesirable antigenic response in the patient. For example, some patients will, over time, develop an allergic reaction to the albumin used as a stabilizer in current commercial formulations. Also, the toxin complexes may induce the immune system of the patient to form neutralizing antibodies, so that larger amounts of toxin are required in subsequent administrations to achieve the same effect. When this happens, subsequent injections must be carefully placed so that they do not release a large amount of toxin into the bloodstream of the patient, which could lead to fatal systemic poisoning. Albumin-containing formulations introduce the risk of, for example, adverse response to albumin, such as greater risk of allergic response, as well as risk of contamination of pathogens for certain blood sources.

In view of the drawbacks associated with current treatments and current botulinum toxin formulations, it would be highly desirable to have an injectable botulinum toxin formulation that is efficacious and stable, but exhibits reduced antigenicity and a lower tendency to diffuse locally after injection. It would also be desirable to use such a botulinum toxin formulation for therapeutic and/or cosmetic purposes, m particular, a stable, longer-acting treatment requiring fewer interventions that produces high, improved response rates without an increase in side effects.

### SUMMARY OF THE INVENTION

The invention relates to methods of using injectable botulinum toxin formulations for administration to an individual to achieve a therapeutic and/or cosmetic benefit having surprisingly high responder rate and long duration of effect.

In one aspect, this invention provides injectable compositions comprising botulinum toxin non-covalently associated with a positively charged carrier. In preferred embodiments, the compositions of the invention possess one or more advantages over conventional commercial botulinum toxin formulations, such as BOTOX® or MYOBLOC®. For instance, in certain embodiments, the compositions may exhibit one or more advantages over conventional injectable botulinum toxin formulations, including reduced antigenicity, a reduced tendency to undergo diffusion into surrounding tissue following injection, increased duration of clinical efficacy, or enhanced potency relative to conventional formulations.

In preferred embodiments, the botulinum toxin component comprises serotype A botulinum toxin having a molecular weight of 150 kDa. The botulinum toxin component may be selected from a botulinum toxin complex (including the 150 kD neurotoxin with accessory proteins found in native complexes produced by C. botulinum), a reduced botulinum toxin complex (including the 150 kD neurotoxin with some, but not all, of the native accessory proteins), and the 150 kD botulinum toxin molecule itself without accessory (non-toxin) proteins.

In preferred embodiments, certain non-native molecules (i.e., molecules not found in botulinum toxin complexes obtained from Clostridium botulinum bacteria) are added to botulinum toxin, botulinum toxin complexes, and in particular reduced botulinum toxin complexes (as defined herein), to improve toxin diffusion through tissues. The non-native molecules associate non-covalently with the toxin and act as penetration enhancers that improve the ability of the toxin to reach target structures after injection. Furthermore, the non-native molecules may increase the stability of the toxin prior to and after injection. By way of example, the penetration enhancers may be positively charged carriers, such as cationic peptides, which have no inherent botulinum-toxin-like activity and which also contain one or more protein transduction domains as described herein.

According to the invention, the positively charged carrier is suitable as a transport system for botulinum toxin, without covalent modification of the toxin molecule, enabling the toxin to be injected with improved characteristics, including longer duration of action and higher responder rate compared to botulinum toxin compositions without the positively charged carrier. The positively charged carrier comprises a positively charged backbone, to which are covalently attached efficiency groups (also referred to as protein transduction domains (PTDs) or cell-penetrating peptides (CPPs)), more preferably at one or both ends of the backbone. In certain embodiments, the efficiency groups are amino acid sequences selected from the group consisting of HIV-TAT or fragments thereof; the PTD of Antennapedia or a fragment thereof; -(gly)n1-(arg)n2 (SEQ ID NO: 5) in which the subscript n1 is an integer of from Oto about 20 and n2 is independently an odd integer from about 5 to about 25; or (gly)p-RGRDDRRQRRR-(gly)q (SEQ ID NO: 1), (gly)p-YGRKKRRQRRR-(gly)q (SEQ ID NO: 2), or (gly)p-RK.KRRQRRR-(gly)q (SEQ ID NO: 3), wherein the subscripts p and q are each independently an integer of from Oto about 20. In one particularly preferred embodiment, the positively charged carrier has the amino acid sequence RKKRRQRRRG-(K)15-GRKKRRQRRR (SEQ ID NO: 4) (also referred to herein as “RTP004”). In still other embodiments, the positively charged carrier has the amino acid sequence YGRKKRRQRRR-G-(K)15-G-YGRKKRRQRRR (SEQ ID NO: 7), RGRDDRRQRRR-G-(K)15-G-RGRDDRRQRRR (SEQ ID NO: 8), or RKKRRQRRR-Q-(K)15-Q-RKKRRQRRR (SEQ ID NO: 11).

In some embodiments, the carrier is provided in the botulinum toxin-containing composition in an amount from about 0.001 to about 1 μg per U of the botulinum toxin component, preferably about 0.01 to about 0.5 μg per U, more preferably about 0.05 to about 0.35 μg per U, or about 0.1 to about 0.3 μg per U, and most preferably about 0.234 μg per botulinum toxin unit. For example, the botulinum toxin-containing composition may contain about 1 to about 20 μg, about 5 to about 15 μg, about 7 to about 12 μg, or about 8 to about 10 μg of the carrier. In one preferred embodiment, the botulinum toxin is in a dosage amount selected from the group consisting of about 20 U to about 60 U, and the carrier is a positively charged carrier present in the composition in an amount selected from about 4.7 to about 14 μg, so as to provide a ratio of about 0.234 μg/U of botulinum toxin.

In some embodiments, the excipient of the botulinum toxin-containing composition comprises one or more additional stabilizing components selected from the group consisting of L-Histidine, L-Histidine hydrochloride, polysorbate 20, and trehalose dihydrate.

In some particularly preferred embodiments, the excipient comprises trehalose dihydrate. For example, per 50 U vial, the excipient may comprise about 1 mg to about 100 mg, about 10 to about 80 mg, about 20 mg to about 60 mg, about 30 mg to about 40 mg, or about 34 mg, about 35 mg, about 36 mg, about 37 mg, about 38 mg, about 39 mg, or about 40 mg trehalose. In some particularly preferred embodiments, the excipient comprises polysorbate 20. For example, per 50 U vial, the excipient may comprise about 0.01 mg to about 1 mg, about 0.05 to about 0.5 mg, about 0.075 mg to about 0.25 mg, about 0.08 mg to about 0.15 mg, or about 0.09 mg, about 0.095 mg, about 0.1 mg, about 0.105 mg, about 0.11 mg, about 0.12 mg, about 0.13 mg, about 0.14 mg, or about 0.15 mg polysorbate 20. In a particularly preferred embodiment, per 50 U, the excipient contains about 36 mg trehalose and about 0.1 mg polysorbate 20.

In one particularly preferred embodiment, referred to as “RT002,” the composition is an injectable formulation, which contains the 150 kD subtype A botulinum toxin molecule, without accessory (non-toxin) proteins, non-covalently associated with a positively charged carrier peptide having the formula RKKRRQRRRG-(K)15-GRKKRRQRRR (SEQ ID NO: 4), and which does not contain accessory proteins or animal-derived components, and as described in WO 2010/151840 (PCT/US2010/040104) to Thompson et al., “Albumin-Free Botulinum Toxin Formulations.” See also, Stone et al., 2011, “Characterization and duration of action of a new botulinum toxin type A formulation” Toxicon, 58:159-167; Garcia-Murray, “Safety and efficacy of RT002, an injectable botulinum toxin type A, for treating glabellar lines: results of a phase 1/2, open-label, sequential dose-escalation study” Dermatol Surg. 2015 January; 41 Suppl 1:S47-55; and Carruthers, et al., Injectable DaxibotulinumtoxinA for the Treatment of Glabellar Lines: A Phase 2, Randomized, Dose-Ranging, Double-Blind, Multicenter Comparison with Onabotulinumtoxin A and Placebo. Dermatol. Surg. 2017; 43: 1321-1331, describing the RT002 formulation; as well as WO 2017/075468 (PCT/US2016/059492) to Ruegg et al., entitled “Injectable Botulinum Toxin Formulations And Methods Of Use Thereof Having Long Duration Of Therapeutic Or Cosmetic Effect;” International Application PCT/US18/48361, to Ruegg entitled “Transmucosal Botulinum Toxin Compositions, Kits, And Methods For Treating Bladder Disorders,” filed Aug. 28, 2018; International Application PCT/US2018/059265, to Rubio entitled “Botulinum Toxin Formulations and Methods of Use Thereof in Plantar Fasciitis,” filed Nov. 5, 2018; and International Application PCT/US18/33397, to Ruegg entitled “Methods of Treatment for Cervical Dystonia,” filed May 18, 2018; each incorporated-by-reference herein in its entirety. RT002 generally is provided in lyophilized form, in a 50 U vial of 150 kDa botulinum toxin A, without accessory (non-toxin) proteins, containing 0.1 mg polysorbate 20, 36 mg trehalose, and 11.7 μg RTP004 as the carrier, to give a mass ratio of peptide:toxin of 51,000:1 in the 50 U vial.

In another aspect, the invention relates to a method for producing a biologic effect by injecting a therapeutically or cosmetically effective amount of a composition of the invention to a subject in need thereof. The biologic effect may include, for example, muscle paralysis, or other effect known to be brought about by administration of botulinum toxin. Such effects include, for example, any one or more of treatment of neurologic pain, headache, or migraine headache, hemifacial spasm, adult onset spasmodic torticollis, anal fissure, blepharospasm, cerebral palsy, strabismus, temporomandibular joint disorder, acne, dystonia, dystonic contractions, hyperhidrosis, or hypersecretion of a gland controlled by the cholinergic nervous system; treatment or management of rhinitis, sinusitis, hyperactive bladder, cervical dystonia, plantar fasciitis; reduction of muscle spasms, and reduction or enhancement of an immune response. In particular examples, the effect is a reduction in the severity of wrinkles, fine lines, furrows, particularly in the face, such as a reduction in the severity of glabellar lines, also known as frown lines.

Methods and compositions described herein deliver the botulinum toxin component in an amount effective to produce the at least one desired biological effect. In certain embodiments, the botulinum toxin is administered from about 1 U to about 300 U, preferably from about 10 U to about 200 U, more preferably from about 20 U to about 100 U or from about 20 U to about 60 U. In preferred embodiments, the botulinum toxin is in a dosage amount selected from the group consisting of about 10 U, about 20 U, about 30 U, about 40 U, about 60 U, about 80 U, and about 80 U, preferably about 40 U.

In another aspect, the invention provides a method of administering botulinum toxin to a subject to achieve an extended duration effect with regard to a desired therapeutic or cosmetic benefit for the subject. In a particular example, the invention provides a method of administering botulinum toxin to achieve an extended duration effect in reducing glabellar lines of an individual. In some embodiments, the method comprises administering by injection a dose of a sterile injectable composition into one or more muscles associated with glabellar lines to achieve the extended duration effect following treatment, that is, sustained efficacy, or a response rate of long duration, following treatment. For example, in some embodiments, administration of the botulinum toxin compositions results in an increased duration of effect, such as an improvement in glabellar lines that lasts longer than treatment with conventional botulinum toxin formulations, thereby extending treatment intervals. Preferred embodiments afford an improvement in glabellar line severity for at least about 3 months through about 11 months, about 5 months through about 10 months, about 6 months through about 10 months, or through about 28 weeks to about 40 weeks. In preferred embodiments, such as using a botulinum toxin dose of 40 U, the duration of effect is at least about 24 weeks, at least about 26, weeks, at least about 28 weeks, at least about 30 weeks, at least about 32 weeks, at least about 34 weeks, at least about 36, weeks, at least about 40 weeks, or at least about 42 weeks, before a second or subsequent treatment dose is administered.

In another aspect, the invention provides a method of achieving a desired therapeutic or cosmetic benefit for the subject, where the method comprises a dosage regimen having multiple treatments with prolonged duration of effect and thus lengthier intervals between successive treatments compared to regimens using conventional botulinum toxin formulations. In a particular embodiment, the invention provides a method of reducing glabellar lines in an individual, where the method comprises a dosage regimen having multiple treatments with prolonged duration of effect and thus lengthier intervals between successive treatments compared to regimens for reducing glabellar lines using conventional botulinum toxin formulations. In particular embodiments, the interval before administering a second or subsequent treatment dose of the composition is greater than or equal to about 26 weeks, about 28 weeks, about 30 weeks, about 32 weeks, about 34 weeks, about 36 weeks, about 38 weeks, about 40 weeks, or greater than or equal to about 42 weeks, following the initial treatment dose or following subsequent treatment doses.

In preferred embodiments, the effect is a reduction in the severity of a glabellar line. In such embodiments, administration may comprise at least one injection into one or more muscle selected from the group consisting of the right corrugator muscle, the left corrugator muscle, and the procerus muscle. For example, in some of these embodiments, about 5 to about 15 U, preferably about 8 U of the botulinum toxin component are injected into the medial aspect of the right corrugator muscle, about 5 to about 15 U, preferably about 8 U are injected into the lateral aspect of the right corrugator muscle; about 5 to about 15 U, preferably about 8 U are injected into the medial aspect of the left corrugator muscle, about 5 to about 15 U, preferably about 8 U are injected into the lateral aspect of the left corrugator muscle; and about 5 to about 15 U, preferably about 8 U are injected into a procerus muscle.

The methods of treatment achieve surprisingly long duration and high responder rates. In particular embodiments, the invention provides methods of reducing a wrinkle, line, or furrow in an individual in need thereof, the method comprising: administering to the individual by injection to one or more muscles or facial structures associated with the wrinkle, line, or furrow of the individual a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)15-GRKKRRQRRR (SEQ ID NO: 4); wherein the botulinum toxin component is administered to the individual in a treatment dose amount of about 20 U to about 60 U, or about 40 U per injection treatment, more preferably wherein the treatment dose is divided amongst injections to one or more muscle selected from the group consisting of the right corrugator muscle, the left corrugator muscle, and the procerus muscle; wherein the positively charged carrier is non-covalently associated with the botulinum component; and wherein the injection of the composition provides a single treatment dose having at least about a 26-week duration of effect in reducing the wrinkle, line, or furrow of the individual, thereby extending treatment interval duration for the individual. The wrinkle, line, or furrow is preferably on the face of the individual, such as a glabellar line.

Accordingly, the methods and uses of the pharmaceutical composition, as described above, allow for methods of treating a glabellar line of an individual in need thereof with injectable botulinum toxin, wherein the method comprises a treatment course having multiple treatment intervals with prolonged duration of effect, and duration of treatment intervals, the treatment course comprising: administering by injection an initial treatment dose of a sterile injectable composition into the glabellar complex of the individual to achieve a reduction in the severity of the glabellar line following the initial treatment with the composition; wherein the composition comprises a pharmaceutically acceptable diluent suitable for injection; a botulinum toxin component that is botulinum toxin of serotype A having a molecular weight of 150 kDa without accessory non-toxin proteins; and a positively charged carrier having the amino acid sequence RKKRRQRRRG-(K)15-GRKKRRQRRR (SEQ ID NO: 4); wherein the botulinum toxin component is administered to the individual in a treatment dose of about 20 U to about 60 U, or about 40 U per injection treatment, more preferably wherein the treatment dose is divided amongst injections to one or more muscle selected from the group consisting of the right corrugator muscle, the left corrugator muscle, and the procerus muscle; wherein the positively charged carrier is non-covalently associated with the botulinum toxin component; wherein the initial treatment dose of the composition administered by injection to the individual provides a duration of effect lasting through at least about 26 weeks; and administering subsequent treatment doses of the composition by injection to the individual at treatment intervals comprising a duration of greater than or equal to about 26 weeks to at least about 40 weeks following the initial treatment dose and between each subsequent treatment dose.

In some embodiments of the methods and uses described in the above two paragraphs, the composition achieves an extended duration of effect for at least about 27 weeks, at least about 28 weeks, at least about 29 weeks, or at least about 30 weeks. In some such embodiments, the positively charged carrier is present in said pharmaceutical composition in an amount of about 0.1 to about 0.3 μg per unit of botulinum toxin component, preferably in an amount of about 0.234 μg per unit of botulinum toxin component. Alternatively or in addition, in some such embodiments, the excipient comprises at least one component selected from the group consisting of L-Histidine, L-Histidine hydrochloride, polysorbate 20, and trehalose dihydrate, preferably trehalose dihydrate.

The duration of effect provided by compositions of the invention, and methods and uses thereof, affords significant advantages compared to the art. Subjects undergoing treatment with compositions containing botulinum toxin consider duration of effect to be of high importance. A long, sustained duration of effect, which can be achieved by even a single treatment with an effective dose according to the invention, permits fewer injections over an entire treatment course for a subject. For example, a prolonged duration of effect from a single injection treatment with a product having clear efficacy and safety, as provided by the inventive compositions and methods herein, offers less discomfort, less cost, and more convenience to subjects undergoing treatments. Accordingly, a product that affords significant and sustained effect, following a single injection treatment, provides a solution to an unmet need in the art for both practitioners and patients. Thus, the compositions and methods of the invention provide a solution to the problem of any one or more of too frequent, painful, and inconvenient treatments, as well as improving patients' overall well-being.

In another aspect, the invention provides for methods of achieving a desired therapeutic or cosmetic benefit for the subject with higher responder rates compared with conventional botulinum toxin formulations. In some embodiments, the invention provides for methods of treating a wrinkle, line, furrow, or glabellar line of an individual in need thereof with injectable botulinum toxin, the method comprising: administering to the individual by injection to one or more muscles or facial structures associated with the wrinkle, line, or furrow of the individual (such as the glabellar complex) a composition comprising: a pharmaceutically acceptable diluent for injection; a botulinum toxin component that is botulinum toxin of serotype A having a molecular weight of 150 kDa without accessory non-toxin proteins; a positively charged carrier having the amino acid sequence RKKRRQRRRG-(K)15-GRKKRRQRRR (SEQ ID NO: 4); wherein the botulinum toxin component is administered to the individual in a treatment dose amount of about 20 U to about 60 U, or about 40 U per injection treatment, more preferably wherein the treatment dose is divided amongst injections to one or more muscle selected from the group consisting of the right corrugator muscle, the left corrugator muscle, and the procerus muscle; wherein the positively charged carrier is non-covalently associated with the botulinum component; and wherein the injection of the composition provides an effect of reducing the severity of the wrinkle, line, furrow, or glabellar line, with an increased rate of response for individuals, each administered the pharmaceutical composition, compared to individuals administered conventional botulinum toxin formulations.

In preferred such embodiments, the effect endures for at least about 4 weeks in over 55%, preferably over 60%, more preferably over 70% of individuals each administered the pharmaceutical composition. In more preferred embodiments, the effect endures for at least about 16 weeks in over 35%, preferably over 50%, more preferably over 70%, of individuals each administered the pharmaceutical composition. In even more preferred embodiments, the effect endures for at least about 24 weeks in over 15%, most preferably over 25%, of individuals each administered the pharmaceutical composition.

This invention also provides kits for preparing formulations containing a botulinum toxin, a botulinum toxin complex, or a reduced botulinum toxin complex and positively charged carrier, or a premix that may in turn be used to produce such a formulation. Also provided are kits that contain means for sequentially administering the botulinum toxin component and the positively charged carrier.

In still another aspect, the invention provides for methods and compositions for use in increasing botulinum toxin duration of action for reducing wrinkles, lines, or furrows in an individual in need thereof by administrating a plurality of successive botulinum toxin treatments, where a first treatment of botulinum toxin composition is administered to the individual by injection to or near a wrinkle, line, or furrow; followed by at least one successive treatment. In preferred embodiments, the first treatment reduces said wrinkle, line, or furrow for at least about 20 weeks, and one or more successive treatments reduce the wrinkle, line, or furrow for a longer duration than achieved following said first treatment. In particular embodiments, the composition comprises a botulinum toxin component in a dose of about 10 U to about 100 U, said botulinum toxin component comprising botulinum toxin of serotype A having a molecular weight of 150 kDa, without accessory (non-toxin) proteins, and a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from Oto 20; wherein the positively charged carrier is non-covalently associated with the botulinum component. In more preferred embodiments, the dose is 40 U and/or the positively charged carrier has the amino acid sequence RKKRRQRRRG-(K)15-GRKKRRQRRR (SEQ ID NO: 4). In still more preferred embodiments, the said positively charged carrier and said botulinum toxin component are present in said pharmaceutical composition in a mass ratio of about 20,000:1 to about 55,000:1; and/or the positively charged carrier is present in the composition in an amount of about 1 to about 50 μg/40U, about 5 to about 25 μg/40; about 10 to about 15 ug/40, or about 12 g per 40 U of said botulinum toxin component. In yet still more preferred embodiments, the excipient comprises at least one component selected from the group consisting of L-Histidine, L-Histidine hydrochloride, polysorbate 20, and trehalose dihydrate, most preferably trehalose dihydrate.

In yet still another aspect, the invention provides for methods and compositions for use in increasing likelihood of achieving a botulinum toxin response of reducing wrinkles, lines, or furrows in an individual in need thereof by administrating a plurality of successive botulinum toxin treatments, where a first treatment of botulinum toxin composition is administered to the individual by injection to or near a wrinkle, line, or furrow; followed by at least one successive treatment that has a greater likelihood of reducing the wrinkle, line, or furrow than the first treatment. In particular embodiments, the composition comprises a botulinum toxin component in a dose of about 10 U to about 100 U, said botulinum toxin component comprising botulinum toxin of serotype A having a molecular weight of 150 kDa, and a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; wherein the positively charged carrier is non-covalently associated with the botulinum component. In more preferred embodiments, the dose is 40 U and/or the positively charged carrier has the amino acid sequence RKKRRQRRRG-(K)15-GRKKRRQRRR (SEQ ID NO: 4). In still more preferred embodiments, the said positively charged carrier and said botulinum toxin component are present in said pharmaceutical composition in a mass ratio of about 20,000:1 to about 55,000:1; and/or the positively charged carrier is present in the composition in an amount of about 1 to about 50 μg/40U, about 5 to about g/40; about 10 to about 15 ug/40, or about 12 μg per 40 U of said botulinum toxin component. In yet still more preferred embodiments, the excipient comprises at least one component selected from the group consisting of L-Histidine, L-Histidine hydrochloride, polysorbate 20, and trehalose dihydrate, most preferably trehalose dihydrate.

In particular embodiments of the inventions described in the above two paragraphs, the wrinkle, line, or furrow is a glabellar line. In such embodiments, administration may comprises at least one injection into one or more muscle selected from the group consisting of the right corrugator muscle, the left corrugator muscle, and the procerus muscle. In still more preferred embodiments, repeat treatments result in fewer side effects following administration of the composition, particularly, reduced eyelid ptosis.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 presents a bar graph showing the required time to return to the baseline digit abduction score (DAS) value (0.4) following repeated administration of either BOTOX® or RT003, a formulation including 150 kDa botulinum toxin type A without accessory proteins (RTT150) and a positively charged carrier having amino acid sequence RKKRRQRRRG-(K)15-GRKKRRQRRR (SEQ ID NO: 4).

FIGS. 2A and 2B: FIG. 2A shows the hind leg of a mouse injected with a dark dye to indicate the portion of a mouse's gastrocnemius muscle that is affected by lateral-to-midline injection. FIG. 2B shows the hind leg of a mouse injected with a dark dye to indicate the portion of a mouse's gastrocnemius muscle that is affected by midline injection.

FIG. 3 presents digital abduction scores (DAS) measured as a function of time following injection of RT003, RTT150, or BOTOX® into either the lateral-to-midline or midline portion of the gastrocnemius muscle of a mouse.

FIGS. 4A and 4B present Kaplan-Meier Curves showing the duration of response in various treatment groups from the clinical study described in Example 5 herein. The Kaplan-Meier Curves represent results from primary efficacy analyses of the clinical study and demonstrate duration of response for at least a I-point improvement from baseline for Investigator Global Assessment-Facial Wrinkle Severity (IGA-FWS) Assessment in the indicated treatment groups. FIG. 4A presents Kaplan-Meier Curves for the Placebo treatment group, the VISTABEL®/BOTOX® 20 U treatment group and the RT002 40 U treatment group. FIG. 4B presents Kaplan-Meier Curves for the Placebo treatment group, the VISTABEL®/BOTOX® 20 U treatment group, the RT002 20 U treatment group, the RT002 40 U treatment group, and the RT002 60 U treatment group.

FIGS. 5A-5C present primary endpoint results from two study arms of a Phase 3 clinical trial described in Example 7 herein. FIG. 5A presents primary endpoints at Week 4 (Intent-to-Treat Population) for the two arms, showing the proportion of subjects who achieved at least a 2-point composite response at maximum frown (*p<0.0001 vs Placebo on a Cochran-Mantel-Haenszel test stratified by study center). FIG. 5B presents Kaplan-Meier Plots of Time to Return to, or Worse Than, Baseline on both IGA-FWS and Patient Facial Wrinkle Severity (PFWS) scales for the two arms (Observed Cases) (ITT). FIG. 5C presents Kaplan-Meier Plots of Time of Maintenance of None or Mild Wrinkle Severity on either IGA-FWS or PFWS Scales of the two arms (Observed Cases) (ITT).

FIGS. 6A-6B compare the two arms of the Phase 3 study with Example 5. FIG. 6A presents a comparison of the primary endpoint at Week 4 of the two arms of the Phase 3 study with that of Example 5's results, comparing percentage of subjects in each ITT population who achieve at least a 2-point composite response at maximum frown. FIG. 6B compares the two arms of the Phase 3 study with Example 5, in terms of the none or mild response on IGA-FWS and PFWS over time.

FIG. 7 presents percent of subjects maintaining none or a mild wrinkles based on these scores over time for each of the two arms, compared to results of Example 5, as well as to other botulinum toxin formulations.

FIG. 8 depicts the two arms of the Phase 3 study's ITT none or mild response on PFWS relative to Example 5.

FIG. 9 depicts the two arms of the Phase 3 study's none or mild response on PFWS relative to Example 5 and relative to abobotulinumtoxinA.

FIG. 10 depicts the two arms of the Phase 3 study's rate of 2:1 point response on IGA-FWS over time relative to Example 5 (PP).

FIG. 11 depicts percent of subjects maintaining at least a 1 point improvement from baseline on IGA-FWS score over time, for each of the two arms and for Example 5.

FIG. 12 depicts percent of subjects maintaining at least a 1 point improvement from baseline on PFWS score over time, for each of the two arms and for Example 5.

FIG. 13 depicts percent of subjects maintaining at least a 1 point improvement from baseline on IGA-FWS and PFWS scores over time, for each of the two arms of the Phase 3 study.

FIG. 14 depicts percent of subjects maintaining none or mild wrinkle severity status (Score of O or 1) on IGA-FWS over time, for each of the two arms (purple and blue lines) and for Example 5 (red line).

FIG. 15 depicts percent of subjects maintaining none or mild wrinkle severity status (Score of O or 1) on IGA-FWS over time, for each of the two arms (purple and blue lines) based on 2-point composite responders at Week 4.

FIG. 16 depicts percent of subjects maintaining none or mild wrinkle severity status (Score of O or 1) on either IGA-FWS of PFWS over time, for each of the two arms (purple and blue lines).

FIG. 17 depicts percent of subjects returning to baseline on both IGA-FWS and PFWS over time, for each of the two arms of the Phase 3 study (purple and blue lines).

FIG. 18 depicts patient global satisfaction rating after treatment for each of two arms of the Phase 3 study.

FIG. 19 presents photographs of a subject showing 2-point improvement on IGA-FWS and PFWS at Week 4 (maximum frown) after treatment; and a I-point sustained duration of effect through Week 24.

FIGS. 20A-20B presents photographs of a different subject showing 2-point improvement on IGA-FWS and PFWS at Week 4 (maximum frown) after treatment; and a I-point sustained duration of effect through Week 24 (FIG. 20B) and Week 32 (FIG. 20B).

FIGS. 21A-B depicts study design for an open label safety study described herein (FIG. 21A) and an overview of this study compared with Example 7's Arms I and 2 (FIG. 21B).

FIG. 22 depicts exemplary injection sites for treatment of mild to moderate glabellar lines with RT002.

FIG. 23 depicts an exemplary schedule of trial assessments for in a clinical trial for evaluating safety of RT002.

FIGS. 24A-24B depict proportion of subjects maintaining improvement m glabellar lines at Week 4 across studies. FIG. 24A depicts subjects who achieve at least a 2-point composite response at Week 4 in the Phase 3 study of Example 8; FIG. 24B depicts subjects who achieve at least a score of +I on both Investigator and Subject GAIS scores at Week 4 across the Phase 3 studies of Example 7, Arms I and 2, and Example 8.

FIGS. 25A-25B depict percent of subjects in Example 7 and Example 8 having wrinkle scores of “none” or “mild” in response to treatment at various time points following the treatment, as assessed by IGA-FWS (FIG. 25A) and PFWS (FIG. 25B).

FIGS. 26A-26B depict percent of subjects in Example 7 and Example 8 versus time following treatment of loss of “none” or “mild” scores on both IGA-FWS and PFWS (FIG. 26A); and of loss to return to baseline on both IGA-FWS and PFWS (FIG. 26B).

FIGS. 27A-27B depict percent of subjects in Example 8 showing a response as assessed by Subject's GAIS (P-GAIS) at maximum frown (FIG. 27A) or by Investigator's GAIS (1-GAIS) at maximum frown (FIG. 27B), over time following treatment.

FIGS. 28A-28B depict photographs of subjects exemplifying a 2-point improvement by IGA-FWS and PFWS at week 4, with sustained duration of effect through Week 16, and a I-point improvement remaining at Week 24.

FIG. 29 depicts median time to loss of none or mild scores on both IGA-FWS and PFWS by subgroup.

by subgroup. FIG. 30 depicts median time to return to baseline on both IGA-FWS and PFWS

### DETAILED DESCRIPTION OF THE INVENTION

This invention relates to botulinum toxin-containing compositions for use in the producing higher responder rates and/or longer duration of effect, or longer duration of action, in therapeutic and/or cosmetic applications of botulinum toxin. In particular, the invention relates to botulinum toxin-containing compositions for use in producing higher responder rates in patients with frown lines, for over an extended period of time, compared with commercially available botulinum toxin preparations, such as BOTOX®.

In one aspect, the compositions used are in sterile injectable formulations that can be administered to an individual by injection, such as by injection into one or more muscles associated with the condition being treated, e.g., one or more muscles associated with moderate to severe glabellar lines. As used herein, the terms compositions and formulations are essentially interchangeable when referring to the compositions and formulations according to the present invention.

### (a) Injectable Compositions

Injectable compositions of this invention, in preferred embodiments, stabilize the toxin and/or enable its delivery through tissues after injection, such that the toxin has one or more advantages, such as reduced antigenicity, a better safety profile, enhanced potency, faster onset of clinical efficacy, higher responder rates, and longer duration of clinical efficacy compared to conventional commercial botulinum toxin formulations (e.g., BOTOX® or MYOBLOC®). In particularly preferred embodiments, the injectable compositions provide an attribute of reduced diffusion or spread from the injection site following injection, thereby localizing the toxin and its effect where desired and decreasing nonspecific or unwanted effects of the toxin at locations distant from the site of injection. The injectable compositions comprise a botulinum toxin in non-covalent association with an effective amount of a positively charged carrier, the carrier comprising a positively charged backbone with covalently attached positively charged “efficiency groups,” which also are referred to as protein transduction domains (PTDs) or cell-penetrating peptides (CPPs). According to the present invention, the positively charged carrier is suitable as a transport system for botulinum toxin, enabling the toxin to be injected with improved characteristics, as discussed above, without covalent modification of the toxin molecule.

The following sections describe the various components of the compositions for use in the present invention.

### (a) The Botulinum Toxin Component

The term “botulinum toxin” as used herein may refer to any of the known types of botulinum toxin (e.g., 150 kD botulinum toxin protein molecules associated with the different serotypes of C. botulinum), whether produced by the bacterium or by recombinant techniques, as well as any types that may be subsequently discovered including newly discovered serotypes, and engineered variants, or fusion proteins. As mentioned above, currently seven immunologically distinct botulinum neurotoxins have been characterized, namely botulinum neurotoxin serotypes A, B, Cl, D, E, F and G, each of which is distinguished by neutralization with type-specific antibodies. The different serotypes of botulinum toxin vary in the animal species that they affect and in the severity and duration of the paralysis they evoke. In preferred embodiments, the composition comprises a botulinum toxin of serotype A

The botulinum toxin serotypes are commercially available, for example, from Sigma-Aldrich (St. Louis, MO) and from Metabiologics, Inc. (Madison, WI), as well as from other sources. At least two types of botulinum toxin, types A and B, are available commercially in formulations for treatment of certain conditions. Type A, for example, is contained in preparations of Allergan, Inc., having the trademark BOTOX®, as well as in preparations of Ipsen Limited, having the trademark DYSPORT®. The original BOTOX® formulation, was prepared by Schantz in 1979 (Schantz et al., “Preparation and characterization of botulinum toxin type A for human treatment” Therapy with Botulinum Toxin. Vol. 109. New York, NY: Marcel Dekker; 1994. pp. 10-24). Type B is contained, for example, in preparations of Elan Pharmaceuticals having the trademark MYOBLOC®. Recombinant botulinum toxin can also be purchased, e.g., from List Biological Laboratories, Campbell, CA

The term “botulinum toxin” can alternatively refer to a botulinum toxin derivative, that is, a compound that has botulinum toxin activity but contains one or more chemical or functional alterations on any part or on any amino acid chain relative to naturally occurring or recombinant native botulinum toxins. For instance, the botulinum toxin may be a modified neurotoxin that is a neurotoxin which has at least one of its amino acids deleted, modified, or replaced, as compared to a native form, or the modified neurotoxin can be a recombinantly produced neurotoxin or a derivative or fragment thereof. For instance, the botulinum toxin may be one that has been modified in a way that, for instance, enhances its properties or decreases undesirable side effects, but that still retains the desired botulinum toxin activity. Alternatively the botulinum toxin used in this invention may be a toxin prepared using recombinant or synthetic chemical techniques, e.g., a recombinant peptide, a fusion protein, or a hybrid neurotoxin, for example prepared from subunits or domains of different botulinum toxin serotypes (See, U.S. Pat. No. 6,444,209, for instance). The botulinum toxin may also be a portion of the overall molecule that has been shown to possess the necessary botulinum toxin activity and, in such case, may be used per se or as part of a combination or conjugate molecule, for instance a fusion protein. Alternatively, the botulinum toxin may be in the form of a botulinum toxin precursor, which may itself be non-toxic, for instance a non-toxic zinc protease that becomes toxic on proteolytic cleavage.

The term “botulinum toxin complex,” or “toxin complex,” as used herein refers to the approximately 150 kD botulinum toxin protein molecule (belonging to any one of botulinum toxin serotypes A-G), along with associated endogenous non-toxin proteins (i.e., hemagglutinin protein and non-toxin non-hemagglutinin protein produced by C. botulinum bacteria). In some embodiments, the botulinum toxin complex need not be derived from C. botulinum bacteria as one unitary toxin complex, but rather may be, for example, botulinum toxin that is recombinantly prepared first and then subsequently combined with the non-toxin proteins.

The term “reduced botulinum toxin complex,” or “reduced toxin complex,” refers to botulinum toxin complexes having reduced amounts of non-toxin protein compared to the amounts naturally found in botulinum toxin complexes produced by C. botulinum bacteria. In one embodiment, reduced botulinum toxin complexes are prepared using any conventional protein separation method to extract a fraction of the hemagglutinin protein or non-toxin non-hemagglutinin protein from botulinum toxin complexes derived from C. botulinum bacteria. For example, reduced botulinum toxin complexes may be produced by dissociating botulinum toxin complexes through exposure to red blood cells at a pH of 7.3, HPLC, dialysis, columns, centrifugation, and other methods for extracting proteins from complexes. Other procedures that can be used are described in, e.g., U.S. Pat. No. 9,469,849 to Ruegg, entitled “Methods And Systems For Purifying Non-Complexed Botulinum Neurotoxin;” WO 2006/096163 to Allergan, Inc., entitled “Animal Product Free System And Process For Purifying ABotulinum Toxin;” and EP 1514556 B1, to Allergan, Inc., entitled “Botulinum toxin pharmaceutical compositions,” each hereby incorporated herein by reference in its entirety. Alternatively, when the reduced botulinum toxin complexes are to be produced by combining synthetically produced botulinum toxin with non-toxin proteins, one may simply add less hemagglutinin or non-toxin, non-hemagglutinin protein to the mixture than what would be present for naturally occurring botulinum toxin complexes.

Any of the non-toxin proteins (e.g., hemagglutinin protein or non-toxin non-hemagglutinin protein or both) in the reduced botulinum toxin complexes may be reduced independently, by any amount. For example, although the amount of endogenous non-toxin proteins may be reduced by the same amount in some cases, this invention also contemplates reducing each of the endogenous non-toxin proteins by different amounts, as well as reducing at least one of the endogenous non-toxin proteins, but not the others.

In certain exemplary embodiments, one or more non-toxin proteins are reduced by at least about 0.5%, 1%, 3%, 5%, 10%, 20%, 30%, 40%, 50%, 60%, 70%, 80%, 90% or 100% compared to the amounts normally found in botulinum toxin complexes. As noted above, C. botulinum bacteria produce seven different serotypes of toxin. Commercial preparations are manufactured with different relative amounts of non-toxin proteins. For example, MYOBLOC® has 5000 U of Botulinum toxin type B per ml with 0.05% human serum albumin, 0.01 M sodium succinate, and 0.1 M sodium chloride. DYSPORT® has 500 U of botulinum toxin type A-hemagglutinin complex with 125 μg albumin and 2.4 mg lactose. In certain embodiments, substantially all of the non-toxin protein (e.g., greater than 95%, 96%, 97%, 98% or 99% of the hemagglutinin protein and non-toxin non-hemagglutinin protein) that would normally be found in botulinum toxin complexes derived from C. botulinum bacteria is removed from the botulinum toxin complex.

Accordingly, in various embodiments, the botulinum toxin component of the present compositions can be selected from a botulinum toxin complex (including the 150 kD neurotoxin with accessory proteins found in native complexes produced by C. botulinum bacteria, as described above), a reduced botulinum toxin complex (including the 150 kD neurotoxin with some, but not all, of the native accessory proteins), and the 150 kD botulinum toxin molecule itself, without accessory (non-toxin) proteins.

In the present composition, botulinum toxin non-covalently associates with a carrier to form a complex without covalent modification to the botulinum toxin molecule. The association between the carrier and the botulinum toxin involves one or more types of non-covalent interaction, non-limiting examples of which include ionic interactions, hydrogen bonding, van der Waals forces, or combinations thereof. See also, e.g., WO 2005/084410 (PCT/US2005/007524), to Dake et al., “Compositions and Methods for Topical Application and Transdermal Delivery of Botulinum Toxins,” further describing how non-covalent association avoids the need to covalently modify the toxin molecule being delivered. The carrier molecules for use in the compositions are described below.

### (a) Carrier Molecules

According to the present invention, a positively charged carrier molecule, having covalently attached efficiency groups, as described herein, has been found suitable as a transport system for botulinum toxin. In certain embodiments, the positively charged carrier will not have other enzymatic or therapeutic biologic activity.

The positively charged carriers enable toxin to be injected with improved delivery to target structures, resulting in decreased diffusion away from injected muscles, such as one or more muscles associated with glabellar lines. Besides enhancing delivery of botulinum toxin, the positively charged carriers may, in certain preferred embodiments, stabilize the botulinum toxin against degradation. In such embodiments, the hemagglutinin protein and non-toxin, non-hemagglutinin protein that are normally present to stabilize botulinum toxin may be reduced or omitted entirely, for example, as described above. Similarly, the exogenous albumin that is normally added during manufacturing may be omitted.

Exemplary positively charged carriers that can be used in injectable compositions of the invention are described, e.g., in WO 2002/007773 (PCT/US2001/023072) to Waugh et al., “Multi-Component Biological Transport Systems;” WO 2005/084410 (PCT/US2005/007524), to Dake et al., “Compositions and Methods for Topical Application and Transdermal Delivery of Botulinum Toxins;” WO 2010/151840 (PCT/US2010/040104) to Thompson eta!, “Albumin-Free Botulinum Toxin Formulations”; WO 2009/015385 (PCT/US2008/071350) to Stone et al., “Antimicrobial Peptide, Compositions, and Methods of Use;” WO 2013/112974 (PCT/US2013/023343) to Waugh et al., “Methods and Assessment Scales for Measuring Wrinkle Severity;” U.S. Pat. No. 9,956,435, to Ruegg et al. “Injectable Botulinum Toxin Formulations;” and WO 2014/066916 (PCT/US2013/67154) to Ruegg et al. “Compositions and Methods for Safe Treatment of Rhinitis;” additional carriers are described, e.g., in US 2016/0166703 A1 to Tan et al., entitled “Carrier Molecule Compositions and Related Methods” and in US 2014/0056811 A1 to Jacob, et al., entitled “New Cell-Penetrating Peptides And Uses Thereof,” each of which is incorporated herein by reference in their entireties.

By the use of the terms “positively charged” or “cationic,” it is meant that the carrier has a positive charge under at least some solution-phase conditions, more preferably, under at least some physiologically compatible conditions. More specifically, “positively charged” or “cationic” means that the group in question contains functionalities that are charged under physiological pH conditions, for instance, a quaternary amine, or that the group contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. Generally, the positively charged carrier comprises a positively charged backbone, described in more detail below.

### (a) Positively Charged Backbones of the Carrier Molecules

The positively charged backbone typically is a chain of atoms, either with groups in the chain carrying a positive charge at physiological pH, or with groups carrying a positive charge attached to side-chains. Generally, the backbone is a linear hydrocarbon backbone which is, in some embodiments, interrupted by heteroatoms selected from nitrogen, oxygen, sulfur, silicon, and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) and can be a homopolymer or a heteropolymer.

In certain preferred embodiments, the positively charged backbone comprises a cationic peptide, such as a polypeptide having multiple positively charged sidechain groups (e.g., lysine, arginine, ornithine, homoarginine, and the like). One of skill in the art will appreciate that when amino acids are used in this portion of the invention, the sidechains can have either the D- or L-form (R or S configuration) at the center of attachment.

As used herein, the term “peptide” refers to an amino acid sequence, but carries no connotation with respect to the number of amino acid residues within the amino acid sequence. Accordingly, the term “peptide” may also encompass polypeptides and proteins. For example, cationic peptide backbones of the invention may comprise from about 5 to about 100 amino acid residues, from about 10 to about 50 amino acid residues, or from about 12 to about 20 amino acid residues. In preferred embodiments, the cationic peptide backbone comprises 10 to 20 amino acids, or 10, 11, 12, 13, 14, 15, 16, 17, 18, 19, or 20 amino acids, preferably being polylysine amino acid residues.

In particularly preferred embodiments, the positively charged backbone is a polylysine. In some embodiments, the polylysine may have a molecular weight that is at least about 100,200,300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention. The polylysine contemplated by this invention can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000.

In some preferred embodiments, the polylysine has a molecular weight from about 1,000 to about 1,500,000 D, from about 2,000 to about 800,000 D, or from about 3,000 to about 200,000 D. In more preferred embodiments, the polylysine has molecular weight from about 100 to about 10,000 D, from about 500 to about 5,000 D, from about 1,000 to about 4,000 D, from about 1,500 to about 3,500 D, or from about 2,000 to about 3,000 D. Especially preferred is a polylysine polypeptide having 10 to 20 lysines (SEQ ID NO: 9), more preferably, 15 lysines. The selection of an appropriate polylysine will depend on the remaining components of the composition and will be sufficient to provide an overall net positive charge to a positively charged carrier.

In another embodiment, the positively charged backbone is a nonpeptidyl polymer, which may be a hetero- or homo-polymer such as a polyalkyleneimine, for example a polyethyleneimine or polypropyleneimine. In some embodiments, the positively charged backbone is a polypropyleneamine wherein a number of the amine nitrogen atoms are present as ammonium groups (tetra-substituted) carrying a positive charge. In another group of embodiments, the backbone has attached a plurality of side-chain moieties that include positively charged groups (e.g., ammonium groups, pyridinium groups, phosphonium groups, sulfonium groups, guanidinium groups, or amidinium groups).

Alternatively, the backbone may comprise amino acid analogs and/or synthetic amino acids. The backbone may also be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the alpha-carbon atoms. As above, a portion or all of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the alpha-carbon locations.

A variety of other backbones can be used employing, for example, steric or electronic mimics of polypeptides wherein the amide linkages of the peptide are replaced with surrogates, such as ester linkages, thioamides (—CSNH—), reversed thioamide (—NHCS—), amino methylene (-NHCH2-) or the reversed methylene amino (-CH2NH-) groups, keto-methylene (-COCH2-) groups, phosphinate (-P02RCH2-), phosphonamidate and phosphonamidate ester (-P02RNH-), reverse peptide (—NHCO—), trans-alkene (—CR═CH—), fluoroalkene (—CF═CH—), dimethylene (-CH2CH2-), thioether (-CH2S-), hydroxyethylene (-CH(OH)CH2-), methyleneoxy (-CH20-), tetrazole (CN4), sulfonamido (-SO2NH-), methylenesulfonamido (-CHRS02NH-), reversed sulfonamide (-NHS02-), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from alpha-amino acids.

In one particularly preferred embodiment, the carrier comprises a relatively short polylysine or polyethyleneimine (PEI) backbone (which may be linear or branched) and which has positively charged efficiency groups covalently attached. When the carrier comprises a relatively short linear polylysine or PEI backbone, the backbone will have a molecular weight of less than 75,000 D, more preferably less than 30,000 D, and most preferably, less than 25,000 D. When the carrier is a relatively short branched polylysine or PEI backbone, however, the backbone will have a molecular weight less than 60,000 D, more preferably less than 55,000 D, and most preferably less than 50,000 D. In more particularly preferred embodiments, the positively charged backbone is a polylysine and positively charged efficiency groups are attached to the lysine at the C- and/or N termini. The efficiency groups are described in detail below.

### (a) Efficiency Groups

Generally, the positively charged backbone has covalently attached one or more efficiency groups (PTDs or CPPs). The efficiency groups can be placed at spacings along the backbone that are consistent in separations or variable. In preferred embodiments, the one or more efficiency groups are attached to either end, or more preferably to each of the two ends, of the backbone of the carrier. Additionally, the length of the efficiency groups can be similar or dissimilar. In embodiments using peptoid backbones, as provided above, efficiency groups can be covalently attached at various atoms or groups of the backbone. For example, the sulfonamide-linked backbones (-SO2NH- and -NHSO2-) can have efficiency groups attached to the nitrogen atoms. Similarly, the hydroxyethylene (-CH(OH)CH2-) linkage can bear efficiency groups attached to the hydroxy substituents. One of skill in the art can readily adapt the other linkage chemistries to provide efficiency groups using standard synthetic methods.

As used herein, an efficiency group is any agent that has the effect of promoting the translocation of the positively charged backbone through a tissue or cell membrane and/or improving delivery of a molecule associated with the backbone to a target site. Non-limiting examples of efficiency groups include HIV-TAT or fragments thereof, the PTD of Antennapedia or a fragment thereof, or -(gly)n1-(arg)n2 (SEQ ID NO: 5) in which the subscript n1 is an integer of from Oto about 20, more preferably Oto about 8, still more preferably about 2 to about 5, and the subscript n2 is independently an odd integer of from about 5 to about 25, more preferably about 7 to about 17, most preferably about 7 to about 13.

In some embodiments, the HIV-TAT fragment does not contain the cysteine-rich region of the HIV-TAT molecule, in order to minimize the problems associated with disulfide aggregation. Preferably, the fragments of the HIV-TAT and Antennapedia PTDs retain the protein transduction activity of the full protein. A preferred efficiency group is, for example, -Gly3Arg7 (SEQ ID NO: 10). Still further preferred efficiency groups, in some embodiments, are those where the HIV-TAT fragment has the amino acid sequence (gly)p-RGRDDRRQRRR-(gly)q (SEQ ID NO: 1), (gly)p-YGRKKRRQRRR-(gly)q (SEQ ID NO: 2), or (gly)p-RK.KRRQRRR-(gly)q (SEQ ID NO; 3), wherein the subscripts p and q are each independently an integer of from Oto about 20, or wherein p and q are each independently the integer 1. In certain preferred embodiments, p is one and q is zero or p is zero and q is one. Preferred HIV-TAT fragments are those in which the subscripts p and q are each independently integers of from 0 to 8, more preferably 0 to 5. In some embodiments, the fragment or efficiency group is attached to the backbone via either the C-terminus or the N-terminus of the fragment or amino acid sequence of the efficiency group.

In some embodiments, the efficiency groups are the Antennapedia (Antp) PTD, or a fragment thereof that retains activity. These are known in the art, for instance, from Console et al., J Biol. Chem. 278:35109 (2003) and a non-limiting example of an Antennapedia PTD contemplated by this invention is the PTD having the amino acid sequence SGRQIKIWFQNRRMKWKKC (SEQ ID NO: 6).

In some embodiments, the efficiency groups comprise a peptide having the amino acid KLAKLAK (SEQ ID NO: 12). Other exemplary efficiency groups include any of the CPPs disclosed in US 2016/0166703 A1 to Tan et al., entitled “Carrier Molecule Compositions and Related Methods” and in US 2014/0056811 A1 to Jacob, et al., entitled “New Cell-Penetrating Peptides And Uses Thereof,” each of which is incorporated herein by reference in their entireties.

In some particularly preferred embodiments, the positively charged carrier is a positively charged peptide having the amino acid sequence RKKRRQRRR-G-(K)15-G-RKKRRQRRR (SEQ ID NO: 4); or a positively charged peptide having the amino acid sequence YGRKKRRQRRR-G-(K)15-G-YGRKKRRQRRR (SEQ ID NO: 7); or a positively charged peptide having the amino acid sequence RGRDDRRQRRR-G-(K)15-G-RGRDDRRQRRR (SEQ ID NO: 8); or positively charged peptide having the amino acid sequence RKKRRQRRR-Q-(K)15-Q-RKKRRQRRR (SEQ ID NO: 11), for use in the compositions and methods of the invention.

### (a) Effective Amounts of the Carrier

For the compositions of the invention, the amount of carrier is selected relative to the amount of botulinum toxin present in a composition to promote stability and/or delivery of the toxin to target sites.

Without wishing to be constrained by theory, it is believed that the positively charged backbones forms a non-covalent electrostatic interaction with anionic surface domains of botulinum toxin to improve penetration to target tissues. It is believed that the positively charged backbone of the carrier also interacts with negatively charged extracellular structures and cell surfaces at the point of administration, such that these interactions restrict the botulinum toxin to the target site, reducing unwanted side effects due to spread to unintended structures. It further is believed that carriers described herein help minimize aggregation of the backbones and the botulinum toxin in therapeutic compositions, which would cause transport efficiency to decrease dramatically. In preferred embodiments, the concentration of carriers in the compositions is sufficient to enhance the delivery of the botulinum toxin to molecular targets such as, motor nerve plates of one or more muscles associated with glabellar lines, e.g., the glabellar complex.

Furthermore, again without wishing to be bound by theory, it is believed that the penetration rate follows receptor-mediated kinetics, such that tissue penetration increases with increasing amounts of penetration-enhancing-molecules up to a saturation point, upon which the transport rate becomes constant. Thus, in preferred embodiments, the amount of carrier in a botulinum toxin-containing composition is selected to be equal, or about equal, to the amount that maximizes penetration rate right before saturation.

In some embodiments, the carrier is provided in the botulinum toxin-containing composition in an amount of about 0.001 to about 1 μg per U of the botulinum toxin component, preferably about 0.01 to about 0.5 μg per U, more preferably about 0.05 to about 0.35 μg per U or about 0.1 to about 0.3 μg per U, and most preferably about 0.234 μg per botulinum toxin unit. In some preferred embodiments, a positively charged carrier is used in an amount greater than about 2.5, greater than about 5, or greater than about 7.5 μg per 40 U of 150 kDa botulinum toxin molecule itself without accessory (non-toxin) proteins. For example, injectable compositions of the present invention may comprise about 0.16 μg/U, about 0.18 μg/U, about 0.2 μg/U, about 0.21 pg/U, about 0.22 μg/U, about 0.23 μg/U, about 0.234 μg/U, about 0.24 μg/U, about 0.25 μg/U, about 0.26 μg/U, about 0.28 μg/U, or about 0.3 μg per U of botulinum toxin.

In some embodiments, the botulinum toxin-containing composition may contain about 1 to about 20 μg, about 5 to about 15 μg, about 7 to about 12 μg, or about 8 to about 10 μg, or about 9 μg of the carrier. In one preferred embodiment, the botulinum toxin is in a dosage amount selected from the group consisting of about 20 U to about 60 U, e.g., about 40 U, and the carrier is a positively charged carrier present in the composition in an amount selected from about 4.7 to about 14 μg, so as to provide a ratio of about 0.234 μg/U of botulinum toxin.

Generally, mass ratio of carrier, preferably RTP004, to botulinum toxin component, preferably the 150 kDa type A toxin without accessory proteins, is about 15,000:1 to about 60,000:1, preferably about 20,000:1 to about 55,000:1, such as about 25,000:1, about 30,000:1, about 35,000:1, about 40,000:1, about 45,000:1, or about 50,000:1. In more particular embodiments, mass ratio of carrier, preferably RTP004, to botulinum toxin component, preferably the 150 kDa type A toxin without accessory proteins, is about 21,000:1, about 22,000:1, about 23,000:1, about 24,000:1, or about 25,000:1; in some other more particular embodiments, mass ratio of carrier, preferably RTP004, to botulinum toxin component, preferably the 150 kDa type A toxin without accessory proteins, is about 49,000:1, about 50,000:1, about 51,000:1, about 52,000:1, or about 53,000:1. For example, per 50 U or per 100 U of toxin, the mass of the peptide carrier may be about 10 μg, about 11 μg, or about 12 μg, such as about 11.7 μgin some particularly preferred embodiments, such as where the toxin amount is about 40 U. In one embodiment, the molar ratio of carrier, preferably RTP004, to botulinum toxin component, preferably the 150 kDa type A toxin without accessory proteins, is a 3:1 molar ratio of carrier:toxin.

In some embodiments, the carrier is provided in the botulinum toxin-containing composition in an amount of about 0.001 to about 1 μg per U of the botulinum toxin component, preferably about 0.01 to about 0.5 μg per U, more preferably about 0.05 to about 0.1 μg per U, and most preferably about 0.075 μg per botulinum toxin unit. In some preferred embodiments, a positively charged carrier is used in an amount greater than about 1.75 μg per 40 U of 150 kDa botulinum toxin molecule without accessory proteins, that is, greater than about 0.04 μg/U, up to about 0.1 μg/U, about 0.2 μg/U, about 0.4 μg/U, about 0.6 μg/U, about 0.8 μg/U, or about 1/μg/U. For example, injectable compositions of the present invention may comprise about 0.045 μg/U, about 0.050 μg/U, about 0.055 μg/U, about 0.060 μg/U, about 0.065 μg/U, about 0.070 μg/U, about 0.075 μg/U, about 0.080 μg/U, about 0.085 μg/U, about 0.090 μg/U, about 0.095 μg/U, or about 0.1 μg per U of botulinum toxin.

In one particular embodiment, the positively charged carrier is RKKRRQRRRG-(K)15-GRKKRRQRRR (SEQ ID NO: 4) (also referred to herein as “RTP004”) and is present at about 3 g per 40 U of botulinum toxin, referring to the 150 kDa toxin protein molecule without accessory proteins. In a particular embodiment of the injectable compositions, botulinum toxin is present in an amount of about 20 U, 40 U, or 60 U (referring to the 150 kDa toxin protein molecule without accessory proteins) and the RTP004 carrier is an amount of about 1.5 μg, about 3.0 μg, or about 4.5 μg, respectively.

### (a) Pharmaceutical Formulations

Pharmaceutical formulations of the compositions for use in achieving high responder rates and/or long duration of a therapeutic or cosmetic effect, generally are prepared by mixing the botulinum toxin component (containing the associated non-toxin proteins, reduced associated non-toxin proteins, or the 150 kD molecule alone without accessory non-toxin proteins) with a carrier described herein, and further with one or more pharmaceutically acceptable excipients or diluents suitable for injection. In their simplest form, they may contain an aqueous pharmaceutically acceptable diluent, such as buffered saline (e.g., phosphate buffered saline). The pharmaceutical formulation also may contain other ingredients typically found in injectable pharmaceutical or cosmeceutical compositions, including a pharmaceutically acceptable carrier, vehicle, or medium that is compatible with the tissues to which it will be applied.

The term “pharmaceutically acceptable” describes compositions or components that are suitable for use in contacting tissues to which the compositions or components will be applied, or for use in patients in general, without undue toxicity, incompatibility, instability, allergic response, and the like. As appropriate, compositions of the invention may comprise any ingredient conventionally used in the fields under consideration, particularly in cosmetics and dermatology.

For example, formulations for injectable use may contain, as appropriate, ingredients typically used in such products, such as antimicrobials, hydration agents, tissue bulking agents or tissue fillers, preservatives, emulsifiers, natural or synthetic oils, solvents, surfactants, detergents, gelling agents, antioxidants, fillers, thickeners, powders, viscosity-controlling agents and water, and optionally including anesthetics, anti-itch actives, botanical extracts, conditioning agents, minerals, polyphenols, silicones or derivatives thereof, vitamins, and phytomedicinals.

In preferred embodiments, the botulinum toxin-containing pharmaceutical formulations do not comprise albumin or other animal protein-derived excipients. As noted above, an exogenous stabilizer (e.g., albumin) is typically added to stabilize conventional botulinum toxin formulations. For instance, in the case of BOTOX®, 0.5 mg of human albumin per 100 U of type A botulinum toxin complex is used to stabilize the complex. In preferred embodiments, the amount of added stabilizer in botulinum toxin compositions herein is less than the amount conventionally added, owing to the ability of the carrier component to act as a stabilizer in its own right. For instance, the amount of added exogenous albumin can be any amount less than the conventional thousand-fold excess of exogenous albumin. In particularly preferred embodiments, no exogenous albumin is added as a stabilizer to the compositions of the invention, thus producing albumin-free botulinum toxin compositions. In some more particularly preferred embodiments, the formulation contains little or no other animal-derived proteins, giving an animal protein-free product.

### (a) Formulations for Injection

Injectable formulations may be in any form suitable for administration by injection and/or for storage until use in such administration. For example, injectable formulations of the compositions used to treat glabellar lines, in accordance with some embodiments of this invention, may include solutions, emulsions (including microemulsions), suspensions, gels, powders, or other typical solid or liquid compositions used in connection with administration by injection to muscle and other target tissues in the face of relevance to the cosmetic use of botulinum toxin.

In preferred embodiments, the compositions of the invention are present in low-viscosity, sterile formulations suitable for injection with a syringe. The compositions of the invention may be in the form of a lyophilized powder that is reconstituted for use, for example, using sterile saline or other known physiologically and pharmaceutically acceptable diluents, excipients, or vehicles, especially those known for use in injectable formulations. In certain embodiments, the lyophilized powder is reconstituted with a liquid diluent to form an injectable formulation with a viscosity of about 0.1 to about 2000 cP, more preferably about 0.2 to about 500 cP, even more preferably about 0.3 to about 50 cP, and still more preferably about 0.4 to about 2.0 cP.

In some embodiments, the injectable formulations may be in the form of controlled-release or sustained-release compositions, which comprise the botulinum toxin component and a positively charged carrier encapsulated or otherwise contained within a material such that they are released within the tissue in a controlled manner over time. For example, the composition comprising the botulinum toxin and positively charged carrier may be contained within matrixes, liposomes, vesicles, microcapsules, microspheres and the like, or within a solid particulate material, all of which is selected and/or constructed to provide release of the botulinum toxin over time. The botulinum toxin and the positively charged carrier may be encapsulated together (i.e., in the same capsule) or separately (i.e., in separate capsules).

In some embodiments, the excipient of the botulinum toxin-containing composition for injection comprises one or more additional stabilizing components. In some embodiments, compositions of the invention comprise liquid (aqueous) formulations comprising a botulinum toxin and a positively charged carrier as described herein, as well as one or more selected from the group consisting of a non-reducing sugar (such as a non-reducing disaccharide or a non-reducing trisaccharide), a non-ionic surfactant, and a physiologically compatible buffer, which is capable of maintaining a suitable pH. Suitable pH's include, for example, pH in the range of pH 4.5 to pH 7.5, or pH 4.5 to pH 6.8, or pH 4.5 to pH 6.5. It is to be understood that a suitable pH also includes the upper and lower pH values in the range, e.g., a pH of 6.5 or a pH of 7.5. Such pharmaceutical formulations are described, for example, in U.S. Pat. No. 9,340,587 to Thompson et al., entitled “Albumin-Free Botulinum Toxin Formulations;” U.S. Pat. No. 9,956,435, to Ruegg et al. entitled “Injectable Botulinum Toxin Formulations;” and WO 2014/066916 (PCT/US2013/67154) to Ruegg et al. “Compositions and Methods for Safe Treatment of Rhinitis,” incorporated by reference in their entireties.

In some embodiments, the concentration of the non-reducing sugar in the liquid composition is in the range of about 10% through about 40% (w/v) and the concentration of the non-ionic surfactant is in the range of about 0.005% through about 0.5% (w/v). The liquid compositions may be dried, preferably by lyophilization, to produce stabilized solid compositions, which may thereafter be reconstituted for use, as described above. Preferably, the dried, e.g., lyophilized, solid compositions are noncrystalline and amorphous solid compositions, and may be in the form of powders.

In certain embodiments, the compositions of the invention contain a non-reducing sugar, which is preferably a disaccharide, non-limiting examples of which include trehalose, including its anhydrous and hydrated forms, or sucrose, as well as combinations thereof. In some embodiments, the hydrated form of trehalose, trehalose dihydrate, is preferable. In other embodiments, the compositions contain a trisaccharide, a non-limiting example of which is raffinose. In general, the concentration of the non-reducing sugar, preferably a disaccharide, is in the range of 10% to 40% (w/v), preferably about 10% to about 25% (w/v), more preferably about 15% to about 20% (w/v). In some preferred embodiments, the concentration of the non-reducing sugar, preferably a disaccharide is about 10%, 11%, 12%, 13%, 14%, 15%, 16%, 17%, 18%, 19% or 20% (w/v).

In general, the compositions of the invention may include any non-ionic surfactant that has the ability to stabilize botulinum toxin and that is suitable for pharmaceutical use. In some embodiments, the non-ionic surfactant is a polysorbate, such as, by way of nonlimiting example, polysorbate 20, polysorbate 40, polysorbate 60, and polysorbate 80. In other embodiments, the non-ionic surfactant is a sorbitan ester, non-limiting examples of which include SPAN® 20, SPAN® 60, SPAN® 65, and SPAN® 80. The non-ionic surfactants Triton®X-100 or NP-40 may also be used. In addition, a combination of the different non-ionic surfactants may be used. In certain preferred embodiments, the non-ionic surfactant is a polysorbate, a poloxamer and/or a sorbitan; polysorbates and sorbitans are particularly preferred. In some embodiments, the non-ionic surfactant is present in the compositions of the invention in the range of about 0.005% to about 0.5%, about 0.01% to about 0.2%, about 0.02% to about 0.1%, or about 0.05 to about 0.08%, inclusive of the upper and lower values. In some preferred embodiments, the compositions of the invention contain a non-ionic surfactant in the amount of (about) 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

In general for injectable formulations herein, any physiologically compatible buffer capable of maintaining appropriate pH is suitable for use. Non-limiting examples of such buffers include salts of citric acid, acetic acid, succinic acid, tartaric acid, maleic acid, and histidine. Non-limiting examples of suitable buffer concentrations include buffer concentrations in the range of about 0.400% to about 0.600%, about 0.450% to about 0.575%, or about 0.500% to about 0.565%. The compositions of the invention may also comprise a mixture of buffer salts, non-limiting examples of which include citrate/acetate, citrate/histidine, citrate/tartrate, maleate/histidine, or succinate/histidine.

A particular composition of the invention is an albumin-free, liquid (aqueous) composition which comprises a botulinum toxin, preferably botulinum toxin of serotype A, or a botulinum toxin A having a molecular weight of 150 kDa and not having associated accessory proteins; a positively charged carrier (e.g., peptide); a non-reducing disaccharide or a non-reducing trisaccharide, preferably a disaccharide, present in a range of 10% through 40% (w/v); a non-ionic surfactant, preferably, a polysorbate or sorbitan ester, present in the range of 0.005% through 0.5% (w/v); and a physiologically compatible buffer, such as citric acid, acetic acid, succinic acid, tartaric acid, maleic acid, or histidine, present in the range of 0.400% to 0.600%; 0.450% to 0.575%, or 0.500% to 0.565%, for maintaining the pH between 4.5. and 7.5.

In particularly preferred embodiments, the pharmaceutical formulation for injection comprises L-Histidine and/or L-Histidine hydrochloride as further stabilizing agents. In particularly preferred embodiments, the excipient comprises trehalose dihydrate, polysorbate 20, L-histidine and L-histidine hydrochloride.

### (a) Use of the Injectable Formulations

In another aspect of the invention, the pharmaceutical formulations described herein are used to produce a biological effect, preferably an extended duration biological effect and/or an effect produced in higher percentages of treated patients, with regard to a desired therapeutic or cosmetic benefit. The pharmaceutical formulation generally is administered to an individual in need thereof to provide a therapeutically or cosmetically effective amount of botulinum toxin. The term “in need” is meant to include both pharmaceutical or health-related needs (e.g., treating conditions involving undesirable facial muscle spasms), as well as cosmetic and subjective needs (e.g., altering or improving the appearance of facial tissue).

Botulinum toxin formulations according to the invention can be delivered by injection (typically using a syringe) to muscles underlying the skin, or to glandular structures within the skin, or other target structures, in an effective amount to produce paralysis, produce relaxation, alleviate contractions, prevent or alleviate spasms, reduce glandular output, or other desired effects. Local delivery of the botulinum toxin in this manner can afford dosage reductions, reduce toxicity and allow more precise dosage optimization for desired effects relative to other injectable or implantable materials.

The compositions of the invention are administered to deliver a therapeutically or cosmetically effective amount of the botulinum toxin. The term “effective amount” or “therapeutically or cosmetically effective amount” as used herein means an amount of a botulinum toxin sufficient to produce the desired muscular paralysis or other biological or aesthetic effect, but that implicitly is a safe amount, i.e., one that is low enough to avoid serious side effects. Desired effects include the relaxation of certain muscles with the aim of, for instance, decreasing the appearance of fine lines and/or wrinkles, especially in the face, or adjusting facial appearance in other ways such as widening the eyes, lifting the corners of the mouth, or smoothing lines that fan out from the upper lip, or the general relief of muscular tension, e.g., in the face or elsewhere.

The botulinum toxin can be administered by injection to muscles or to other skin-associated or target tissue structures. Generally, a formulation according to the invention is injected at a location or locations where an effect associated with botulinum toxin is desired. The administration may be made, for example, to the face, legs, shoulders, back (including lower back), axilla, palms, feet, neck, face, groin, dorsa of the hands or feet, elbows, upper arms, knees, upper legs, buttocks, torso, pelvis, or any other parts of the body where administration of the botulinum toxin is desired.

Administration of the injectable botulinum toxin-containing compositions of this invention may be carried out to treat any condition for which prevention of synaptic transmission of or release of acetylcholine would confer a therapeutic or cosmetic benefit. For example, the conditions that may be treated by the compositions according to the invention include, without limitation, neurologic pain, migraine headache or other headache pain, overactive bladder, rhinitis, sinusitis, acne, dystonia, dystonic contractions (whether subjective or clinical), hyperhidrosis (whether subjective or clinical), and hypersecretion of one or more glands controlled by the cholinergic nervous system. The compositions of this invention may also be used for reducing or enhancing immune response, or treatment of other conditions for which administration of botulinum toxin by injection has been suggested or performed. In some embodiments, the biological effect is reducing undesirable facial muscle spasms, or other muscular spasms.

In particular embodiments, the botulinum toxin-containing composition is administered to reduce severity of wrinkles, fine lines, furrows, particularly in the face, such as a reduction in the severity of glabellar lines. The compositions of the invention are particularly suited for treatment of fine lines, such as facial fine lines and glabellar lines of a subject.

The glabella is the skin between the eyebrows and above the nose. Glabellar lines or glabellar facial lines (often called “frown lines”) are those vertical lines that develop between the eyebrows and may appear as a single vertical line or as two or more lines and may also appear angled towards the inner corners of the eyebrows. When a person frowns, the muscles of the lower forehead contract in a downward direction causing the skin between the eyebrows to crease. Lines are formed by the repeated action of frowning due to the lack of elasticity in the skin. More specifically, glabellar lines arise from the lateral corrugator and vertical procerus muscles in the face. The corrugator depresses the skin creating a vertical line, i.e., a furrow, surrounded by ridges of tensed muscle (i.e., frown lines). Age, sun exposure, and genetics are contributing factors. Botulinum toxin is used to block the nerve impulses, temporarily paralyzing muscles that cause the frown lines, giving the skin a smoother, more refreshed appearance.

The severity of a subject's glabellar lines can be assessed by various criteria, by either a health professional and/or the subject. For assessment by someone other than the subject, an Investigator Global Assessment-Facial Wrinkle Severity (IGA-FWS) rating score system can be used. Specifically, an IGA-FWS rating score of (0) is used to indicate no facial wrinkle severity; an IGA-FWS rating score of (1) is used to indicate mild facial wrinkle severity; an IGA-FWS rating score of (2) is used to indicate moderate facial wrinkle severity; and an IGA-FWS rating score of (3) is used to indicate severe facial wrinkle severity. As appreciated by the skilled practitioner, a photo guide exhibits the grades of wrinkle severity and can be used by the person conducting the assessment, during prior training and/or as a reference during the assessment.

A Patient Facial Wrinkle Severity (PFWS) can be used by the subject himself/herself to assess his/her facial wrinkle severity. Subjects can complete the PFWS based on maximum frown to assess the severity of the glabellar lines. The subject may look in a mirror or at a picture of himself/herself. The PFWS rating score system is as follows: a PFWS rating score of (0) indicates no wrinkle severity, with associated description of “no wrinkles;” a PFWS rating score of (1) indicates mild wrinkle severity, with associated description of “very shallow wrinkles;” a PFWS rating score of (2) indicates moderate wrinkle severity, with associated description of “moderate wrinkles;” and a PFWS rating score of (3) indicates severe wrinkle severity, with associated description of “deep wrinkles.”

Visual appearance of wrinkles, lines, or furrows also may be assessed using a number of other or additional criteria, such as the Patient Global Aesthetic Improvement Scale (GAIS). This scale can be used by the subject or a person other than the subject to assess visual appearance of a glabellar line, such as at maximum frown and/or at rest after maximum frown, to determine improvement from baseline condition. In some embodiments, a 7-point severity GAIS is used, where a rating score of “3” indicates “very much improved;” a rating score of “2” indicates “much improved; “a rating score of” 1” indicates “improved;” a rating score of “0” indicates “no change;” a rating score of “−1” indicates “worse;” a rating score of “−2” indicates “much worse;” and a rating score of “−3” indicates “very much worse.” A subject may use a mirror or photograph of himself/herself for the assessment.

In some embodiments, methods of the invention produce an effect of reduction in severity of moderate and/or severe wrinkles, according to one or more of the scales described above, preferably moderate to severe wrinkles. A reduction in severity may be a 1 point, 2 point, or 3 point improvement, or more, in one or more scales described herein.

Safety and efficacy of the injectable formulations described herein also may be assessed by evaluating one or more cranial nerves following injection to the face, head, or neck region, such as one or more cranial nerves that innervate target and adjacent musculature. For example, in some embodiments, cranial nerves II-VII are evaluated, where cranial nerve II is the optic nerve, cranial nerve III is the oculomotor nerve, cranial nerve IV is the trochlear nerve, cranial nerve V is the trigeminal nerve, cranial nerve VI is the abducens nerve, and cranial nerve VII is the facial nerve.

Safety and efficacy of the injectable formulations described herein also may be assessed by evaluating facial muscle strength following injection to the face, head, or neck area. Facial muscle strength can evaluated using the Medical Research Council Scale for Assessment of Muscle Power (MRC). The MRC is a reliable and validated scale for assessing muscle weakness and aids the investigation of peripheral nerve injuries. For example, strength of the orbicularis oculi (eyelid), lateral brow elevators, and lateral orbicularis zygomaticus muscles on each side of the face may be evaluated. In the MRC Scale, a rating of (0) corresponds to “no movement;” a rating of (1) corresponds to “flicker perceptible in the muscle;” a rating of (2) corresponds to “movement only if gravity is eliminated;” a rating of (3) corresponds to “can move limb against gravity;” a rating of (4) corresponds to “can move against gravity and some resistance exerted by examiner;” and a rating of (5) corresponds to “normal power.”

### (a) Dosage and Administration

Methods and compositions described herein deliver the botulinum toxin component in a dose or amount effective to achieve at least one biological effect, preferably an extended duration biological effect, with regard to a desired therapeutic or cosmetic benefit, more preferably achieving the effect in a higher percentage of subjects receiving treatment. Generally, therapeutically or cosmetically effective amounts are provided as doses in botulinum toxin units contained in the pharmaceutical formulations for administration by injection, in accordance with the present invention.

In certain embodiments using injectable formulations, the botulinum toxin is administered to provide from about 1 U to about 300 U, preferably from about 10 U to about 200 U, more preferably from about 20 U to about 100 U; or more specifically, from about 10 U to about 30 U, from about 30 U to about 50 U, or about 50 U to about 70 U per injection treatment. In preferred embodiments, the botulinum toxin-containing compositions of the invention are administered to a subject in need thereof by injection, so as to provide a dose greater than about 10 U, about 20 U, about 30 U, about 40 U, about 60 U, or about 80 U of the botulinum toxin. In preferred embodiments, the composition is administered by injection in an amount that provides 20 U or at least 20 U; 30 U or at least 30 U; 40 U or at least 40 U; 50 U or at least 50 U; 60 U or at least 60 U; 70 U or at least 70 U; 80 U or at least 80 U; 90 U or at least 90 U; or 100 U or at least 100 U of botulinum toxin per injection treatment. Amounts or doses between the foregoing amounts or doses are also contemplated, for example, 25 U or at least 25 U; 35 U or at least 35 U; 45 U or at least 45 U, and the like. In particularly preferred embodiments, the botulinum toxin is in a dosage amount selected from the group consisting of about 10 U, about 20 U, about 30 U, about 40 U, about 60 U, and about 80 U, more preferably botulinum toxin of serotype A, most preferably the 150 kDa molecule of serotype A botulinum toxin without accessory proteins. Generally, an amount of about 100 μg/kg of the 150 kDa molecule of botulinum toxin A without accessory proteins will correspond to about 16 U/kg, in liquid injectable formulations of the present invention. In a particular embodiment, the composition is administered by injection as a single treatment dose in an amount that provides 20, 30 U, 35 U, 40 U, 45 U, 50 U, or 60 U of botulinum toxin to result in a decrease in wrinkles and/or facial lines, such as a reduction in the severity of glabellar lines.

An “injection treatment” refers to a single treatment that may comprise one or more injections to the patient, e.g., all within a single patient visit, such as a series of injections administered within seconds or minutes of each other; and/or administered in the same general area of the patient's body (e.g., the glabellar complex) through one or more injection sites in relative close proximity (e.g., about 1 cm, about 2 cm, about 3 cm, about 4 cm, or about 5 cm apart).

In general, methods and procedures for measuring the activity of botulinum toxin, i.e., units (U) of botulinum toxin activity, are known to and practiced by those having skill in the art. Briefly, median lethality assays (LDso assays) in mice are conventionally used to estimate the number of units of botulinum toxin with a high degree of precision. Doses of all commercially available botulinum toxins are expressed in terms of units of biologic activity. By way of example, one unit of botulinum toxin corresponds to the calculated median intraperitoneal lethal dose (LD50) in female Swiss-Webster mice. See, Hoffman, R O. et al., 1986, Int. Ophthalmol. Clin., 26:241-50, as well as DePass, L. R., 1989, Toxicol. Letters, 49:159-170; and Pearce, L. B. et al., 1994, Toxicol. Appl. Pharmacol., 128:69-77, which also describe lethality assays in the art.

More particularly, a suitable method for determining botulinum toxin units for a botulinum toxin component of the compositions of the invention is as follows: Forty-eight (48) female CD-I mice weighing 17-23 μgrams are randomly assigned to six doses of the test article (1.54, 1.31, 1.11, 0.95, 0.80, and 0.68 U/0.5 mL), eight (8) animals per dose group. The test article refers to the botulinum toxin preparation or sample being assayed or tested. The animals are housed eight per cage and are weighed within 24 hours of dosing with the test article. On the day of dosing, the test article is diluted to the appropriate concentrations in isotonic saline (0.9% NaCl). Each animal is administered 0.5 mL of diluted test article via intraperitoneal injection. After injection, mice are returned to the cage and fatalities are recorded daily for three days. Lethality is scored 72 hours post injection and the results are analyzed by probit or logistic analysis to derive the LDso value relative to a reference standard that is assessed using the same dosing regimen. By way of example, the reference standard is a specifically qualified and calibrated lot of the same composition of the invention that is used for comparison to derive relative potency of the test article. The determined LDso value is then corrected for the cumulative dilutions performed to assign a relative potency value for the neat (undiluted) test article.

Alternatives to LDso testing include assays using neuronal cell lines or endopeptidase assays, which avoid testing in animals (see, e.g., Sesardic et al., “Alternatives to the LD50 assay for botulinum toxin potency testing: Strategies and progress towards refinement, reduction and replacement” Proc. 6^{th} World Congress on Alternatives & Animal Use in the Life Sciences, Aug. 21-25, 2007, 14 Special Issue, pp 581-585). Such methods may be used, in addition or instead of LDso assays, for determining botulinum toxin units for a botulinum toxin component of the compositions of the invention.

The pharmaceutical formulations of the invention may contain a therapeutically or cosmetically effective amount of the botulinum toxin for application as a single-dose treatment, such as a single injection or single injection treatment. Alternatively, the pharmaceutical formulations may be more concentrated, e.g., for dilution at the place of administration, or may contain therapeutically or cosmetically effective amounts of the botulinum toxin for use in multiple applications, such as use in a specified number of sequential applications over a course of treatment or over a period of time. Local delivery of the botulinum toxin, as described herein, may afford dosage reductions, reduce toxicity, and allow more precise dosage optimization for desired effects relative to conventional botulinum toxin formulations. In preferred embodiments, the dose (e.g., in units and the volume) is selected to optimize delivery of the toxin to target receptor/neurotransmitter containing muscle or fascial/periosteal nociceptors. Optimization may be based, for example, on dose dilution distribution principles (see, e.g., U.S. Pat. Nos. 8,632,768 and 8,506,970).

Generally, the botulinum toxin-containing pharmaceutical formulation is administered to a patient in need thereof by injection into, or near to, one or more of the muscles associated with the condition to be treated, in a patient in need thereof. Administration “near to” or “at” a structure means administration close enough to the structure to allow the botulinum toxin component to readily diffuse to the structure, taking into consideration the reduced diffusion of the botulinum toxin compositions disclosed herein. For example, administration near to the glabellar complex means administration within about 0.05 mm, about 0.1 mm, about 0.5 mm, about 1 mm, about 5 mm, about 10 mm, about 15 mm, or about 20 mm of the structure being targeted. In certain embodiments, ultrasound or other visualization techniques may be used to guide placement of the injection, or injection fractions.

In particular embodiments, the condition is severe to moderate glabellar lines, and the pharmaceutical formulation is injected into one or more muscles of the glabellar complex. The formulation may be injected by applying finger pressure on the superior medial orbital rim while advancing the needle through the skin into the underlying muscle. In more particular embodiments, a treatment dose is divided amongst injections to one or more muscle selected from the group consisting of the right corrugator muscle, the left corrugator muscle, and the procerus muscle, administered during a given injection treatment. For example, the total treatment dose may be divided in half, thirds, quarters, fifths, sixths, sevenths, eighths, ninths, or tenths, etc., and specific dose amounts are injected into different target structures.

For example, in one embodiment, a fraction of the total treatment dose is injected into each of a number of injection sites on the forehead, between the eyebrows of the subject undergoing treatment. For example, a dose of about 10 U to about 20 U, about 10 U to about 15 U, or about 13 U of botulinum toxin is injected into each of the left corrugator muscle, the right corrugator muscle, and the procerus muscle. As another example, a dose of about 10 U to about 30 U, about 15 U to about 20 U, or about 16 U of botulinum toxin is injected into each of the left corrugator muscle and the right corrugator muscle, and a dose of about 5 U to about 15 U, about 7 U to about 10 U, or about 8 U of botulinum toxin is injected into the procerus muscle. In an especially preferred embodiment, two injections are applied into each corrugator muscle, and one injection into the procerus muscle, for a total of 5 injections, for example, a dose of about 5 U to about 15 U, about 7 U to about 10 U, or about 8 U of botulinum toxin is injected into each of the medial aspect of the left corrugator muscle, the lateral aspect of the left corrugator muscle, the medial aspect of the right corrugator muscle, the lateral aspect of the right corrugator muscle, and the procerus muscle.

In some embodiments, the patient to be treated is 65 years of age, at least 65 years old, or over 65 years old. For example, the patient may be 65, 66, 68, 70, 72, 73, 75, 77, 78, 80 years, or older.

Most preferably, the formulations are administered by or under the direction of a physician or other health care professional. They may be administered in a single treatment or in a series of treatments over time. Because of its nature, the botulinum toxin preferably is administered at an amount, application rate, and frequency that will produce the desired result without producing serious adverse or undesired results.

### (a) Extended Duration

In another aspect, the invention provides methods and uses of the pharmaceutical formulations, described herein, to achieve an extended duration of effect. In preferred embodiments, formulations described herein are used to administer botulinum toxin to a subject in need thereof to produce an extended duration therapeutic effect compared to treatments using conventional botulinum toxin formulations. In some embodiments, the method comprises administering by injection a therapeutically or cosmetically effective dose of a sterile injectable formulation, as described herein, preferably into one or more muscles or other structures associated with glabellar lines, or other lines or wrinkles, to achieve the extended duration effect following the injection treatment. In preferred embodiments, administration of the botulinum toxin compositions results in an increased duration of effect, such as an improvement in at least biological effect associated with a therapeutic or cosmetic benefit, that lasts longer than treatment with conventional botulinum toxin formulations, thereby allowing lengthier intervals between treatments.

Particularly preferred embodiments afford a therapeutic and/or cosmetic effect, in particular, a reduction in glabellar line severity, for about 3 months through about 11 months, about 5 months through about 10 months, about 6 months through about 10 months, or about 16 weeks through about 24 weeks, or about 28 weeks through about 40 weeks. In preferred embodiments, the duration of effect is at least about 16 weeks, at least about 20 weeks, at least about 24 weeks, at least about 6 months, at least about 28 weeks, at least about 7 months, at least about 30 weeks, at least about 32 weeks, at least about 8 months, at least about 34 weeks, at least about 36, weeks, at least about 9 months, at least about 40 weeks, at least about 10 months, or at least about 42 weeks, before a second or subsequent treatment dose is administered. In particular embodiments, the interval before administering a second or subsequent treatment dose of the composition is greater than or equal to 26 weeks, 28 weeks, 30 weeks, 32 weeks, 34 weeks, 36 weeks, 38 weeks, 40 weeks, or greater than or equal to 42 weeks, following the initial treatment dose or following subsequent treatment doses.

Duration of effect regarding reducing lines, wrinkles, or furrows may be assessed by any measure described herein or known in the art, or a combination thereof. For example, any one or more measures discussed in the Examples herein, in particular Example 7, e.g., for primary and/or secondary endpoints, may be used to measure duration of effect. For example, a reduction in a glabellar line may be considered to endure until the time the line returns to baseline, before initial treatment; or may be considered to endure until one more “points” are lost, based on one or more measures for assessing wrinkle severity, as described herein; or may be considered to endure as long as scores of 0 or 1 (none or mild wrinkles) are maintained, again based on one or more measures for assessing wrinkle severity, as described herein.

In another aspect, the invention provides methods and uses of the pharmaceutical formulations, described herein, in a treatment regimen for achieving a biological effect associated with a therapeutic or cosmetic benefit, where intervals between one or more successive treatments are longer than those in a treatment regimen for same using conventional botulinum toxin formulations, such as where multiple treatments are used to maintain a treatment goal. For example, the invention provides, in some embodiments, a method of reducing the severity of glabellar lines in a subject, where the method comprises a treatment course having multiple treatments with prolonged duration of effect and, accordingly, lengthier intervals between successive treatments compared to regimens using conventional botulinum toxin formulations (i.e., formulations not containing a carrier molecule, as described herein). For example, products containing botulinum toxin without a carrier described herein typically provide an effect for less than 6 months, such as only for about 3-4 months.

In particular embodiments, the interval before administering a second or subsequent treatment dose of the botulinum toxin-containing composition is greater than or equal to at least about 26 weeks, at least about 28 weeks, at least about 30 weeks, at least about 32 weeks, at least about 34 weeks, at least about 36 weeks, at least about 38 weeks, at least about 40 weeks, or at least about 42 weeks, or equal to about 42 weeks, following the initial treatment dose or following subsequent treatment doses. A median duration between doses may be 23 weeks, at least 23 weeks, or greater than 23 weeks; 24 weeks, at least 24 weeks, or greater than 24 weeks; 25 weeks, at least 25 weeks, or greater than 25 weeks; 26 weeks, at least 26 weeks, or greater than 26 weeks; 27 weeks, at least 27 weeks, or greater than 27 weeks; 28 weeks, at least 28 weeks, or greater than 28 weeks; 30 weeks, at least 30 weeks, or greater than 30 weeks, e.g., up to about one year.

In particular, one or more of the results in the above paragraphs of this section are obtained in embodiments comprising administering by injection a dose of a sterile injectable formulation into at least one muscle or facial structure associated with the wrinkle, facial line, or furrow (such as the glabellar complex) to achieve a reduction in its severity following treatment, preferably following a first treatment. In some such embodiments, the composition comprises a pharmaceutically acceptable diluent for injection; botulinum toxin, such as botulinum toxin A, preferably the 150 kDa molecule without accessory proteins; and a positively charged carrier comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2), or (gly)p-RK.KRRQRRR-(gly)q (SEQ ID NO: 3), wherein the subscripts p and q are each independently an integer of from Oto 20, preferably where the carrier comprises or consists of the ammo acid sequence of RKKRRQRRRG-(K)15-GRKKRRQRRR (SEQ ID NO: 4). More preferably, the botulinum toxin is administered by injection to the individual in a single treatment dose in an amount that provides about 10 U to about 100 U, or about 20, 40, or 60 U botulinum toxin, most preferably 40 U per injection treatment. In a particular example, the pharmaceutical formulation further comprises a non-reducing disaccharide, such as sucrose or trehalose, preferably trehalose dihydrate; a non-ionic surfactant, such as polysorbate 20, polysorbate 40, polysorbate 60, polysorbate 80, or a sorbitan ester; and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5. to pH 7.5, e.g., in w/v amounts as described herein; and/or the carrier:toxin mass ratio is about 20,000:1 to about 55,000:1, more preferably about 51,000:1. In still more preferred embodiments, the formulation is albumen-free and/or free of animal protein and the pharmaceutical composition comprises 0.1 mg polysorbate 20, 36 mg trehalose dihydrate, and 8 μg, 9 μg, 10 μg, 11 μg, 12 μg, 13 μg, 14 μg, and 15 μg, most preferably 11.7 μg, RTP004 per 50 U of the 150 kDa type A toxin without accessory proteins, and the treatment dose is 40 U.

In some such embodiments, administration comprises about 3-7 injections in a single treatment, preferably 5 injections into the glabellar complex, such as where about 5 U to about 15 U, about 7 U to about 10 U, or about 8 U of the botulinum toxin component are injected into each of the medial aspect of the right corrugator muscle, the lateral aspect of the right corrugator muscle, the medial aspect of the left corrugator muscle, the lateral aspect of the left corrugator muscle, and the procerus muscle, in a single treatment (e.g., all within a single visit, in a series of injections within seconds to minutes of each other), more preferably using about 0.01 to about 1 ml/injection, about 0.02 to about 0.8 mL/injection, about 0.04 to about 0.6 mL/injection, about 0.06 to about 0.4 mL/injection, about 0.08 to about 0.2 mL/injection, or about 0.1 mL/injection. See also Example 7.

In preferred embodiments, such as those recited above, the therapeutic or cosmetic effect may have at least about a 6 month to about a 10 month duration, or about a 26 week to 40 week duration, before a second or a subsequent treatment dose is administered. More preferably, the extended therapeutic or cosmetic effect is achieved following treatment by a single injection treatment of the composition, such as by a pharmaceutical formulation as described in the paragraph above. Still more preferably, treatment regimens as described herein provide sustained improvement in the appearance of facial lines, such as sustained reduction in the severity of glabellar lines.

In a particular embodiment, a single dose of a composition of the invention containing a positively charged carrier as described and 150 kDa botulinum toxin A, without accessory proteins, in a dosage amount of 40 U provides a long duration effect in treating glabellar lines, e.g., for at least 24 weeks, 25 weeks, 26 weeks, 27 weeks, or 28 weeks, or 30, 32, 34 or 36 weeks, e.g., up to about one year.

In a course of treatment according to the present invention, the injectable formulations may be administered at less frequent intervals following an initial treatment dose based on the extended duration of effect afforded by the therapeutically and cosmetically effective doses of the compositions and methods of the invention as described herein. For example, the compositions of the invention may be administered (or dosed) to an individual in need about twice per year (about every 6 months), or fewer times that twice a year, such as, e.g., every 7 months, 8 months, 9 months, or 10 months, or 11 months, or once a year, by the practice of the methods of the invention. In a particular embodiment, an individual is administered a dose of a composition of the invention twice per year. A median duration between doses may be 23 weeks, 24 weeks, 25 weeks, 26 weeks, 27 weeks, 28 weeks, 29 weeks, or 30 weeks, or 31 weeks, 32 weeks, 33 weeks, 34 weeks, 35 weeks, or 36 weeks, depending on the therapeutic or cosmetic treatment and/or the desire for treatment as determined by the individual being treated.

### (a) Higher Responder Rates

Along with extended duration of effect or in alternative embodiments, it also has been surprisingly found that the biological effect occurs in a higher proportion of individuals receiving treatment compared with commercially available botulinum toxin preparations, such as BOTOX®. That is, in some preferred embodiments, the therapeutic or cosmetic effect, following administration of a botulinum toxin-containing formulation disclosed herein, occurs in and/or lasts for an extended duration of time for a higher proportion of individuals receiving the botulinum toxin pharmaceutical formulation compared with conventional botulinum toxin formulations such as formulations lacking a positively charged carrier, as described herein.

For example, administration of a pharmaceutical composition described herein may produce a biological effect, with therapeutic or cosmetic benefit, such as a reduction in severity of a wrinkle, line (e.g., a glabellar line), or furrow, that endures for at least about 4 weeks in 40-90% of individuals each administered the formulation. In preferred embodiments, the response is maintained, or the effect endures, for about 4 weeks in about 55 to about 60%, about 65% to about 70%, or about 65% to about 75% of individuals each administered the pharmaceutical composition. In some embodiments, the response is maintained, or the effect endures, for at least about 4 weeks in at least over about 55%, over about 56%, over about 58%, over about 60%, over about 62%, over about 65%, over about 66%, over about 68%, over about 70%, over about 72%, over about 73%, or over about 75% of individuals each administered the pharmaceutical formulation, as described herein (e.g., as in Example 7), up to about 75%, about 80%, or about 90% of individuals each administered the pharmaceutical formulation.

In more preferred embodiments, the effect, such as a reduction in severity of a wrinkle, line (e.g., a glabellar line), or furrow, endures for at least about 16 weeks in about 30% to about 80% of individuals each administered the formulation, such as enduring for about 16 weeks in about 35% to about 40%, about 40% to about 50%, or about 50% to about 70% of individuals each administered the pharmaceutical composition. In some embodiments, the response is maintained, or the effect endures, for at least about 16 weeks in at least over about 35%, over about 36%, over about 38%, over about 40%, over about 43%, over about 45%, over about 47%, over about 50%, over about 53%, over about 55%, over about 57%, over about 60%, over about 63%, over about 65%, over about 68%, more preferably over 70%, over 73%, or over 75%, of individuals each administered the pharmaceutical formulation, as described herein (e.g., as in Example 7), up to about 75%, about 80%, or about 90% of individuals each administered the pharmaceutical formulation.

In even more preferred embodiments, the effect, such as a reduction in severity of a wrinkle, line (e.g., a glabellar line), or furrow, endures for at least about 24 weeks in about 10% to about 30% of individuals each administered the formulation, such as enduring for about 24 weeks in about 15% to about 20%, or about 20% to about 30%, of individuals each administered the pharmaceutical composition. In some embodiments, the response is maintained, or the effect endures, for at least about 24 weeks in at least over about 15%, over about 16%, over about 18%, over about 20%, over about 22%, over about 23%, over about 25%, over about 27%, or over about 30%, of individuals each administered the pharmaceutical formulation, as described herein (e.g., as in Example 7), up to about 30%, about 40%, or about 50% of individuals each administered the pharmaceutical formulation.

Thus, methods and compositions for use, as described above, afford methods of reducing severity of a wrinkle, line (e.g., a glabellar line), or furrow of an individual in need thereof with an increased rate of response for individuals, each administered the pharmaceutical composition, compared to individuals administered conventional botulinum toxin formulations. In particular, one or more of the results in the above paragraphs of this section are obtained in embodiments comprising administering by injection a dose of a sterile injectable formulation into at least one muscle or facial structure associated with the wrinkle, facial line, or furrow (such as the glabellar complex) to achieve a reduction in its severity following treatment, preferably following a first treatment. In some such embodiments, the composition comprises a pharmaceutically acceptable diluent for injection; botulinum toxin, such as botulinum toxin A, preferably the 150 kDa molecule without accessory proteins; and a positively charged carrier comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2), or (gly)p-RKKRRQRRR-(gly)q (SEQ ID NO: 3), wherein the subscripts p and q are each independently an integer of from Oto 20, preferably where the carrier comprises or consists of the amino acid sequence of RKKRRQRRRG-(K)15-GRKKRRQRRR (SEQ ID NO: 4). More preferably, the botulinum toxin is administered by injection to the individual in a single treatment dose in an amount that provides about 10 U to about 100 U, or about 20, 40, or 60 U botulinum toxin, most preferably 40 U per injection treatment. In a particular example, the pharmaceutical formulation further comprises a non-reducing disaccharide, such as sucrose or trehalose, preferably trehalose dihydrate; a non-ionic surfactant, such as polysorbate 20, polysorbate 40, polysorbate 60, polysorbate 80, or a sorbitan ester; and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5. to pH 7.5, e.g., in w/v amounts as described herein; and/or the carrier:toxin mass ratio is about 20,000:1 to about 55,000:1, more preferably about 51,000:1. In still more preferred embodiments, the formulation is albumen-free and/or free of animal protein and the pharmaceutical composition comprises 0.1 mg polysorbate 20, 36 mg trehalose dihydrate, and 8 μg, 9 μg, 10 μg, 11 μg, 12 μg, 13 μg, 14 μg, 15 μg, most preferably 11.7 μg RTP004, per 50 U of the 150 kDa type A toxin without accessory (non-toxin) proteins, and treatment dose is 40 U.

In some such embodiments, administration comprises about 3-7 injections in a single treatment, preferably 5 injections into the glabellar complex, such as where about 5 U to about 15 U, about 7 U to about 10 U, or about 8 U of the botulinum toxin component are injected into each of the medial aspect of the right corrugator muscle, the lateral aspect of the right corrugator muscle, the medial aspect of the left corrugator muscle, the lateral aspect of the left corrugator muscle, and the procerus muscle, in a single treatment (e.g., all within a single visit, in a series of injections within seconds to minutes of each other), more preferably using about 0.01 to about 1 ml/injection, about 0.02 to about 0.8 mL/injection, about 0.04 to about 0.6 mL/injection, about 0.06 to about 0.4 mL/injection, about 0.08 to about 0.2 mL/injection, or about 0.1 mL/injection. See also Example 7.

### (a) Kits

This invention also contemplates the use of a variety of delivery devices for administering botulinum toxin-containing compositions described herein across skin to produce a biological effect, preferably an extended duration effect in a high percentage of subjects receiving treatment, with regard to a desired therapeutic or cosmetic benefit. Such devices may include, without limitation, a needle and syringe, or may involve more sophisticated devices capable of dispensing and monitoring the dispensing of the composition, and optionally monitoring the condition of the subject in one or more aspects (e.g., monitoring the reaction of the subject to the substances being dispensed).

It should be noted that the choice of materials for the construction of the device is important. Preferred materials for the construction of delivery devices are those that do not lead to a loss of activity of the botulinum toxin/carrier composition, either through degradation or unwanted adsorption of the botulinum toxin on a surface of the device. Such undesired behavior has been observed, for example, when botulinum toxin/carrier in an aqueous solution contacts polypropylene surfaces, but not when the botulinum toxin/carrier solution contacts polyvinyl chloride (PVC) surfaces.

In some embodiments, the compositions can be pre-formulated and/or pre-installed in a delivery device. This invention also contemplates embodiments wherein the compositions are provided in a kit that stores one or more components separately from the remaining components. For example, in certain embodiments, the invention provides for a kit that separately stores the botulinum toxin component and the carrier in separate containers (e.g., first and second containers) for combining at or prior to the time of application. The amount of carrier to botulinum toxin will depend on which carrier is chosen for use in the composition in question.

For example, the amount of carrier to botulinum toxin may be provided in a ratio selected from the group consisting of about 0.01 μg/U, about 0.02 μg/U, about 0.04 μg/U, about 0.06 μg/U, about 0.08 μg/U, about 0.1 μg/U, about 0.12 μg/U, about 0.14 μg/U, about 0.15 μg/U, about 0.16 μg/U, about 0.18 μg/U, about 0.20 μg/U, about 0.22 μg/U, about 0.23 μg/U, about 0.234 μg/U, about 0.24 μg/U, about 0.25 μg/U, about 0.26 μg/U, about 0.28 μg/U, about 0.3 μg/U, about 0.32 μg/U, about 0.34 μg/U, about 0.36 μg/U, about 0.38 μg/U, or about 0.4 μg/U botulinum toxin, preferably the 150 kDa type A toxin without accessory proteins, and more preferably where the carrier is RTP004. In particular embodiments, botulinum toxin is provided in an amount of about 40 U (referring to the 150 kDa toxin protein molecule of type A without accessory proteins) and the RTP004 carrier is provided an amount of about 6 μg, about 7 μg, about 8 μg, about 9 μg, about 10 μg, about 11 μg, or about 12 μg.

In other embodiments, the carrier is RTP004 and is provided in an amount greater than about 1.75 μg per 40 U of 150 kDa botulinum toxin molecule without accessory proteins, that is, greater than about 0.04 μg/U. For example, the amount of carrier to botulinum toxin may be provided in a ratio selected from the group consisting of about 0.045 μg/U, about 0.050 μg/U, about 0.055 μg/U, about 0.060 μg/U, about 0.065 μg/U, about 0.070 μg/U, about 0.075 μg/U, about 0.080 μg/U, about 0.085 μg/U, about 0.090 μg/U, about 0.095 μg/U, or about 0.1 μg per U of botulinum toxin. In particular embodiments, botulinum toxin is provided in an amount of about 20 U, 40 U, or 60 U (referring to the 150 kDa toxin protein molecule without accessory proteins) and the RTP004 carrier is provided an amount of about 1.5 μg, about 3.0 μg, or about 4.5 μg, respectively.

The invention also contemplates approaches for administering the botulinum toxin component to a subject or patient in need thereof, in which a therapeutically effective amount of botulinum toxin is administered in conjunction with a carrier, as described herein. By “in conjunction with” it is meant that the two components (botulinum toxin and carrier) are administered in a combination procedure, which may involve either combining them prior to administration to a subject, or separately administering them, but in a manner such that they act together to provide the requisite delivery of a therapeutically effective amount of the toxin. The botulinum toxin may be stored in dry form in a syringe or other dispensing device and the carrier may be injected or topically applied before application of the toxin so that the two act together, resulting in the desired tissue penetration enhancement and/or other improved characteristics over conventional botulinum toxin formulations, as detailed above. In that sense, the two substances (carrier and botulinum toxin) act in combination or interact to form a composition or combination in situ. Accordingly, the invention also includes a kit with a device for dispensing botulinum toxin and a liquid, gel, or the like, that contains the carrier and that is suitable for topical application or injection to the target tissue. Kits for administering the compositions of the inventions, either under direction of a health care professional or by the patient or subject, may also include a custom applicator suitable for that purpose.

(a) Repeat Treatments with Long Duration, High Responder Rates, and Continued Safety

In still another aspect, the invention is directed to methods and compositions for use in administrating a plurality of successive botulinum toxin treatments, that consistently provide results and advantages, as described above. In particular, a botulinum toxin component in non-covalent association with a positively charged carrier can be administered more than once to an individual to reduce severity of a wrinkle, line, or furrow, particular in the face, e.g., a glabellar line. In some preferred embodiments, a subsequent treatment achieves a longer duration of therapeutic or cosmetic effect, such as longer duration of reduction in wrinkles, lines, or furrows, compared with duration of effect following a first or earlier treatment. In some preferred embodiments, a subsequent treatment achieves a response in a higher percent of individuals receiving treatment, compared with the response rate following a first or earlier treatment, such as a higher percent of subjects showing reduction in glabellar lines and/or maintaining the reduction for extended periods of time. In some preferred embodiments, a subsequent treatment achieves a response with fewer side effects compared with side effects associated with a first or earlier treatment, such as fewer adverse events following repeat for glabellar lines. In more preferred embodiments, a subsequent treatment achieves two or more improvements over a first or earlier treatment, such as achieving both longer duration and fewer adverse events, both longer duration and higher likelihood of response, higher likelihood of response and fewer adverse events. In most preferred embodiments, a subsequent treatment achieves longer duration, fewer adverse events, and higher likelihood of response in an individual receiving the repeat treatment, compared to that individual's response following a prior treatment.

In particular embodiments, the composition for use in achieving one or more improvements upon repeat treatment comprises a botulinum toxin component in a dose of about 1 U to about 300 U, preferably from about 10 U to about 200 U, more preferably from about 20 U to about 100 U; or more specifically, from about 10 U to about 30 U, from about 30 U to about 50 U, or about 50 U to about 70 U per injection treatment. In preferred embodiments, the botulinum toxin-containing compositions of the invention are administered to a subject in need thereof by injection, so as to provide a dose greater than about 10 U, about 20 U, about 30 U, about 40 U, about 60 U, or about 80 U of the botulinum toxin. In preferred embodiments, the composition is administered by injection in an amount that provides 20 U or at least 20 U; 30 U or at least 30 U; 40 U or at least 40 U; 50 U or at least 50 U; 60 U or at least 60 U; 70 U or at least 70 U; 80 U or at least 80 U; 90 U or at least 90 U; or 100 U or at least 100 U of botulinum toxin per injection treatment. Amounts or doses between the foregoing amounts or doses are also contemplated, for example, 25 U or at least 25 U; 35 U or at least 35 U; 45 U or at least 45 U, and the like. In particularly preferred embodiments, the botulinum toxin is in a dosage amount selected from the group consisting of about 10 U, about 20 U, about 30 U, about 40 U, about 60 U, and about 80 U, more preferably botulinum toxin of serotype A, most preferably the 150 kDa molecule of serotype A botulinum toxin. Generally, an amount of about 100 pg/kg of the 150 kDa molecule of botulinum toxin A without accessory proteins, will correspond to about 16 U/kg, in liquid injectable formulations of the present invention.

Repeat treatments may use the same dose or different doses, e.g., escalating doses or decreasing doses at different treatments. In a particular embodiment, the composition is administered by injection in repeat treatments that each provide approximately the same dose, such as a dose set forth in the paragraph above, preferably a dose of about 20 U, about 40 U, or about 60 U of type A botulinum toxin having 150 kDa MW, without accessory proteins, to result in a decrease in wrinkles, lines, or furrows, such as a reduction in the severity of glabellar lines, for an extended duration of time, exceeding duration following a first or prior treatment.

In particular, one or more of the results in the above paragraphs of this section are obtained in embodiments comprising successively administering by injection a dose of a sterile injectable formulation into at least one muscle or facial structure associated with the wrinkle, facial line, or furrow (such as the glabellar complex). In some such embodiments, the composition comprises a pharmaceutically acceptable diluent for injection; botulinum toxin, such as botulinum toxin A, preferably the 150 kDa molecule without accessory proteins; and a positively charged carrier comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2), or (gly)p-RKKRRQRRR-(gly)q (SEQ ID NO: 3), wherein the subscripts p and q are each independently an integer of from Oto 20, preferably where the carrier comprises or consists of the amino acid sequence of RKKRRQRRRG-(K)15-GRKKRRQRRR (SEQ ID NO: 4). More preferably, the botulinum toxin is administered by injection to the individual in a treatment dose in an amount that provides about 10 U to about 100 U, or about 20, 40, or 60 U botulinum toxin, most preferably 40 U per injection treatment. In a particular example, the pharmaceutical formulation further comprises a non-reducing disaccharide, such as sucrose or trehalose, preferably trehalose dihydrate; a non-ionic surfactant, such as polysorbate 20, polysorbate 40, polysorbate 60, polysorbate 80, or a sorbitan ester; and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5. to pH 7.5, e.g., in w/v amounts as described herein; and/or the carrier:toxin mass ratio is about 20,000:1 to about 55,000:1, more preferably about 51,000:1. In still more preferred embodiments, the formulation is albumen-free and/or free of animal protein and the pharmaceutical composition comprises 0.1 mg polysorbate 20, 36 mg trehalose dihydrate, and 8 μg, 9 μg, 10 μg, 11 μg, 12 μg, 13 μg, 14 μg, or 15 μg, most preferably 11.7 μg, RTP004 per 50 U of the 150 kDa type A toxin without accessory (non-toxin) proteins, and the treatment dose is 40 U.

In some such embodiments, administration comprises about 3-7 injections in a single treatment, preferably 5 injections into the glabellar complex, such as where about 10 U to about 30 U, about 15 U to about 20 U, or about 16 U of the botulinum toxin component are injected into each of the right corrugator muscle and the left corrugator muscle, and about 5 U to about 15 U, about 7 U to about 10 U, or about 8 U of the botulinum toxin component into the procerus muscle, in a given treatment (e.g., all within a single visit, in a series of injections within seconds to minutes of each other), more preferably using about 0.01 to about 1 ml/injection, about 0.02 to about 0.8 mL/injection, about 0.04 to about 0.6 mL/injection, about 0.06 to about 0.4 mL/injection, about 0.08 to about 0.2 mL/injection, or about 0.1 mL/injection. See also Example 8.

(a) Further Extended Duration following Repeat Treatment

In some particular embodiments, the invention provides methods and compositions for use in increasing botulinum toxin duration of action for reducing wrinkles, lines, or furrows in an individual in need thereof by administrating a plurality of successive botulinum toxin treatments, where a first treatment of botulinum toxin composition is administered to the individual by injection to or near a wrinkle, line, or furrow; followed by at least one successive treatment. In preferred embodiments, the first treatment reduces said wrinkle, line, or furrow for at least about 20 weeks, and one or more successive treatments reduce the wrinkle, line or furrow for longer duration than achieved following the first treatment.

In particular embodiments, the wrinkle, line, or furrow to be reduced in severity is a glabellar line. In such embodiments, administration may comprise at least one injection into one or more muscle selected from the group consisting of the right corrugator muscle, the left corrugator muscle, and the procerus muscle. In more particular, embodiments, administration is as described above, in Example 7 or Example 8 below.

Duration of effect in reducing glabellar lines following repeat treatments generally can be assessed according to any methods known in the art or described herein, including measures disclosed above for assessing during after a first treatment and/or in Example 8. Particularly preferred embodiments afford a reduction in glabellar line severity, for at least about 20 weeks, at least about 24 weeks, at least about 6 months, at least about 28 weeks, at least about 7 months, at least about 30 weeks, at least about 32 weeks, at least about 8 months, at least about 34 weeks, at least about 36, weeks, at least about 9 months, at least about 40 weeks, at least about 10 months, or at least about 42 weeks, before a second or subsequent treatment dose is administered. In particular embodiments, the interval before administering a second or subsequent treatment dose of the composition is greater than or equal to 26 weeks, 28 weeks, 30 weeks, 32 weeks, 34 weeks, 36 weeks, 38 weeks, 40 weeks, or greater than or equal to 42 weeks, following the initial treatment dose or following subsequent treatment doses.

Generally, a subsequent or successive treatment is administered after the period of duration of effect, e.g., after severity of a subject's glabellar lines increase from being not visible or mild, to appearing moderate or severe, or after returning to their baseline before treatment. In some embodiments, subsequent or successive treatment is administered earlier, e.g., before the wrinkle becomes visible, or before the wrinkle returns to its appearance before the first treatment. Accordingly, in some embodiments, a successive or subsequent treatment is administered about 12 weeks to about 36 weeks, about 16 weeks to about 34 weeks, about 16 weeks to about 32 weeks, about 18 weeks to about 36 weeks, about 20 weeks to about 36 weeks, about 21 weeks to about 35 weeks, about 22 weeks to about 34 weeks, about 23 weeks to about 33 weeks, about 24 weeks to about 32 weeks, about 25 weeks, to about 31 weeks, and about 26 weeks to about 30 weeks.

In some embodiments, the patient to be treated is 65 years of age, at least 65 years old, or over 65 years old. For example, the patient may be 65, 66, 68, 70, 72, 73, 75, 77, 78, 80 years, or older. In preferred such embodiments, the extended duration of effect and/or the interval between successive treatments may be any of the period of time disclosed above or longer, in particular, about 26 to about 52 weeks, about 27 to about 50 weeks, about 28 to about 48 weeks, about 29 to about 46 weeks, about 30 to about 44 weeks, about 31 to about 42 weeks, or about 32 to about 40 weeks.

It will be appreciated that increasing duration of effect following subsequent treatments allows for longer and longer intervals of time between successive treatments. For example, a course of treatment with compositions comprising botulinum toxin non-covalently associated with a positively charged carrier, may have a first interval between first and second treatments that is shorter than the second interval between second and third treatments, which may be equal to or shorter than the third interval between third and fourth treatments, etc. For example, the treatment course may comprise a first interval of about 12 weeks and a second of greater than 12 weeks, such as about 14 weeks, about 16 weeks, or about 20 weeks. The treatment course may comprise a first interval of about 16 weeks and a second of greater than 16 weeks, such as about 18 weeks, about 20 weeks, or about 22 weeks. The treatment course may comprise a first interval of about 20 weeks and a second of greater than 20 weeks, such as about 22 weeks, about 24 weeks, or about 26 weeks. The treatment course may comprise a first interval of about 24 weeks and a second of greater than 24 weeks, such as about 26 weeks, about 28 weeks, or about 30 weeks. Intervals over the course of treatment may increase over one, two, three or more cycles of treatment; or only over the first few cycles, such as only over cycles one, two, and three, or only over cycles one and two.

In some embodiments, the interval between subsequent botulinum toxin treatments is about 12 weeks to about 36 weeks, about 16 weeks to about 34 weeks, about 16 weeks to about 32 weeks, about 18 weeks to about 36 weeks, about 20 weeks to about 36 weeks, about 21 weeks to about 35 weeks, about 22 weeks to about 34 weeks, about 23 weeks to about 33 weeks, about 24 weeks to about 32 weeks, about 25 weeks, to about 31 weeks, and about 26 weeks to about 30 weeks. In preferred embodiments, the interval between subsequent botulinum toxin treatments is greater than about 20 weeks to about 36 weeks, greater than about 22 weeks to about 34 weeks, greater than about 24 weeks to about 32 weeks, or greater than about 26 weeks to about 30 weeks. In more preferred embodiments, the subsequent interval is at least one of the second interval, the third interval, the fourth interval, and the fifth interval.

It further will be appreciated that longer intervals translate to fewer treatments over a period of time, such as over the period for which the subject desires treatment.

### (a) Increased Responder Rate Following Repeat Treatment

In some particular embodiments, the invention provides for methods and compositions for use in increasing likelihood of achieving a botulinum toxin response of reducing wrinkles, lines, or furrows in an individual in need thereof by administrating a plurality of successive botulinum toxin treatments, where a first treatment of botulinum toxin composition is administered to the individual by injection to or near a wrinkle, line, or furrow; followed by at least one successive treatment that has a greater likelihood of reducing the wrinkle, line, or furrow than the first treatment. In preferred embodiments, the wrinkle, line, or furrow is a glabellar line. In particular, the response in reducing wrinkles, lines, or furrows is an extended duration of response, such that an individual has increased likelihood of maintaining botulinum toxin response for an extended period following repeat treatments. For example, a subject may have an increased likelihood of maintaining a reduction in glabellar lines, as assessed by one or more measures described herein, for 4 weeks following a subsequent treatment, compared to likelihood of maintaining the reduction for 4 weeks following a first treatment. More preferably, a subject has an increased likelihood of maintaining a reduction in glabellar lines, as assessed by one or more measures described herein, for 8 weeks, for 12 weeks, for 16 weeks, for 20 weeks, for 24 weeks, for 28 weeks, for 32 weeks, or for 36 weeks following a subsequent treatment, compared to likelihood of maintaining the reduction for 8 weeks, for 12 weeks, for 16 weeks, for 20 weeks, for 24 weeks, for 28 weeks, for 32 weeks, or for 36 weeks, respectively, following the first treatment.

In some embodiments, the reduction in the wrinkle, line, or furrow, such as a glabellar line, endures for at least about 4 weeks in at least 80% of individuals following administration of the first treatment dose, and/or for at least 85% of individuals following administration of the second or subsequent treatment dose. In preferred embodiments, the reduction in the wrinkle, line, or furrow, such as a glabellar line, endures for at least about 4 weeks in at least 85% of individuals following administration of the first treatment dose, and/or for at least 90% of individuals following administration of the second or subsequent treatment dose. In more preferred embodiments, reduction in the wrinkle, line, or furrow, such as a glabellar line, endures for at least about 4 weeks in at least 90% of individuals following administration of the first treatment dose, and/or for at least 95% of individuals following administration of the second or subsequent treatment dose (see, e.g., Example 8).

For example, administration of a pharmaceutical composition described herein may produce a reduction in severity of a wrinkle, line (e.g., a glabellar line), or furrow, that endures for at least about 4 weeks in 40-90% of individuals following a first treatment, and endures for at least about 4 weeks in a greater percentage following a subsequent treatment. In preferred embodiments, the response is maintained, or the effect endures, for about 4 weeks in about 55 to about 60%, about 65% to about 70%, or about 65% to about 75% of individuals each administered the first treatment, and endures for about 4 weeks in about 60 to about 65%, about 70% to about 75%, or about 70% to about 80% of individuals each administered the second treatment. In some embodiments, the response is maintained, or the effect endures, for at least about 4 weeks in at least over about 55%, over about 56%, over about 58%, over about 60%, over about 62%, over about 65%, over about 66%, over about 68%, over about 70%, over about 72%, over about 73%, or over about 75%, up to about 80% of individuals each administered the first treatment, and for at least about 4 weeks in at least over about 57%, over about 58%, over about 60%, over about 62%, over about 64%, over about 67%, over about 68%, over about 70%, over about 72%, over about 74%, over about 75%, or over about 77% of individuals each administered the second treatment (see, e.g., Example 8), up to about 80%, about 85%, or about 90% of individuals each administered the second treatment of the pharmaceutical formulation.

### (a) Reduced Side Effects Following Repeat Treatment

In some particular embodiments, the invention provides for methods and compositions for use in reducing side effects associated with botulinum toxin administration in reducing wrinkles, lines, or furrows in an individual in need thereof by administrating a plurality of successive botulinum toxin treatments, where a first treatment of botulinum toxin composition is administered to the individual by injection to or near a wrinkle, line, or furrow; followed by at least one successive treatment that results in fewer adverse effects than the first treatment. In preferred embodiments, the wrinkle, line, or furrow is a glabellar line.

The side effect, or adverse event, associated with botulinum toxin administration is any adverse event that is a definite, probable, or possible treatment-emergent or treatment-related adverse event, in terms of its relation to administration of botulinum toxin, e.g., as described in Example 8. The adverse event may be mild, moderate, severe, or serious, e.g., as described in Example 8. In particular, use of a composition comprising botulinum toxin non-covalently associated with a positively charged carrier may reduce side effects (adverse events) generally associated with distant spread of the toxin. Such adverse events include, without limitation, accommodation disorder, are flexia, aspiration, blurred vision, botulism, eyelid function disorder, eyelid ptosis, facial palsy, facial paresis, fourth cranial nerve paresis, peripheral nerve palsy, peripheral paralysis, pelvic floor muscle weakness, pneumonia aspiration, pupillary reflex impaired, bradycardia, brow ptosis, bulbar palsy, constipation, cranial nerve palsies, cranial nerve paralysis, diaphragmatic paralysis, diplopia, dry mouth, dysarthria, dysphagia, dysphonia, dyspnea, extraocular muscle paresis, paresis, gastrointestinal disorders, quadriparesis, headaches, hemiparesis, hypoglossal nerve paresis, hyporeflexia, hypotonia, monoparesis, muscular weakness, paralysis, paralysis flaccid, paralytic ileus, paraparesis, paresis cranial nerve, respiratory failure, respiratory arrest, respiratory depression, speech disorder, third cranial nerve paresis, trigeminal nerve paresis, urinary retention, vocal cord paralysis, vocal cord paresis, and xerophthalmia (dry eyes).

In preferred embodiments, repeated treatment according to methods and uses herein leads to in fewer occurrences and/or reduced severity of one or more of such adverse events compared with the initial treatment. In a more preferred embodiment, frequency and/or severity of eyelid ptosis is reduced following a subsequent treatment compared with eyelid ptosis following a first treatment.

It is understood that the following examples and embodiments described herein are for illustrative purposes and that various modifications or changes in light thereof will be suggested to persons skilled in the art and are to be included within the spirit and purview of this application and scope of the appended claims. Numerical values qualified by “about” herein also refer to the exact numerical value.

All publications, patents, and published patent applications cited herein are hereby incorporated by reference in their entireties for all purposes.

### EXAMPLES

### (i) Example 1: Duration Of Local Muscle Paralysis In A Murine Model

This example compares the duration of local muscle paralysis in mice injected with either RT003 or BOTOX®. RT003 is an exemplary injectable formulation according to the invention that contains type A botulinum toxin (purified to remove all endogenous non-toxin proteins) and positively charged carrier with the sequence RKKRRQRRRG-(K)15-GRKKRRQRRR. BOTOX® also contains type A botulinum toxin, but exogenous albumin is added to stabilize the type A botulinum toxin molecule.

The muscle paralysis was measured using digit abduction score (DAS) assay as reported by Aoki, K. R. in “A comparison of the safety margins of botulinum neurotoxin serotypes A, B, and Fin mice”, Toxicon 2001; 39(12):1815-1820. In the DAS assay, a mouse is briefly suspended by its tail to cause a characteristic startle response in which the mouse extends its hind limbs and abducts its hind digits. The extent to which the mouse is able to exhibit this startle response is scored on a five-point scale (from 0-4), with zero representing a normal startle response and four representing maximal reduction in digit abduction and leg extension. The scoring is done by an observer with no knowledge of the extent to which the subject mouse has been treated with neurotoxin. The baseline score using the DAS assay was determined to be 0.4 for an untreated population of animals.

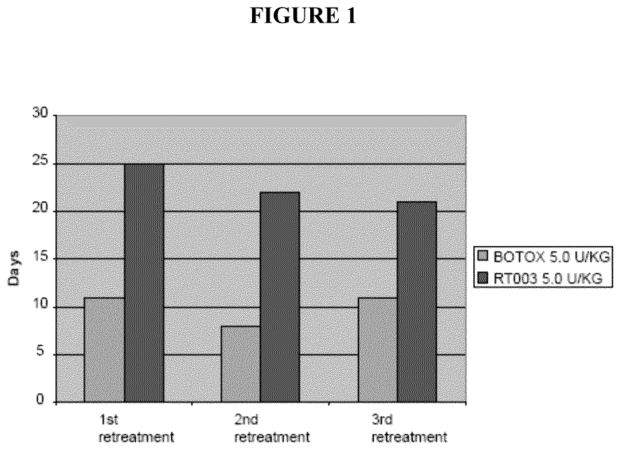

The study reported in this example involved ten animals (5 mice in RT003 group and 5 mice in BOTOX® group). Each of the animals was injected three times with the respective botulinum toxin formulation (i.e., RT003 or BOTOX®), with a 40-day period in between each dosing. After injection, the number of days that all of the animals in each test group was above the 0.4 baseline of the DAS assay was counted. The results, shown in FIG. 1, indicate that the DAS assay score for the RT003-treated group stayed above the 0.4 baseline value for 25, 22, and 21 days, following the first, second, and third treatment, respectively. In contrast, the DAS assay score for the BOTOX®-treated group stayed above the 0.4 baseline value for 11, 8, and 11 days, following the first, second, and third treatment, respectively.

These DAS assay data indicate that local muscle paralysis caused by the RT003 formulation lasts approximately twice as long as the local muscle paralysis caused by BOTOX®. This result has important implications for therapeutic uses of RT003 and other injectable botulinum toxin-containing compounds according to the invention. In particular, by using injectable compositions according to the invention, one can significantly reduce the frequency of follow-up injections required to maintain a particular cosmetic or therapeutic effect caused by the botulinum toxin. In turn, the reduced frequency of application can result in better long-term efficacy, as the subject is less prone to develop antibodies to the botulinum toxin.

### (i) Example 2: Injectable Botulinum Toxin Formulations With an Improved Safety Profile

Over the last few decades, botulinum toxin has found use as a therapeutic agent for treating a variety of conditions, including wrinkles, hyperhidrosis, and muscle spasms. However, as botulinum toxin is the most potent naturally occurring toxin known to humans, improper administration of the toxin can be extremely dangerous. For instance, accidental systemic delivery of botulinum toxin can lead to paralysis, difficulty breathing, and even death. Moreover, even if botulinum toxin were properly delivered to a localized region of the body as a part of a therapeutic treatment, the toxin has a natural tendency to diffuse over time, thereby increasing the risk of unwanted paralysis in other parts of the body. For example, when botulinum toxin is injected around the eyes to treat wrinkles, it may diffuse to the muscles that control the movement of the eyelids. If this happens, the eyelid muscles may become partially paralyzed, leading to a well-known condition known as “eyelid droop,” in which the eyelid is partially closed and interferes with normal vision.

One aspect of this invention is to provide injectable botulinum toxin formulations with an improved safety profile compared to currently available commercial botulinum toxin formulations. In preferred embodiments, the injectable botulinum toxin formulations have a reduced tendency to diffuse after injection. In this way, certain preferred formulations of the invention permit more accurate delivery of botulinum toxin, dramatically reducing unwanted side effects associated with uncontrolled local diffusion of botulinum toxin.

This example reports a comparative study of the tendency of botulinum toxin in various formulations to diffuse following injection. The study involved three botulinum toxin formulations: (1) BOTOX®; (2) RT003, a buffered and stabilized solution containing the 150 kD type A botulinum toxin molecule itself without accessory proteins, non-covalently associated with a positively charged carrier having the formula RKKRRQRRRG-(K)15-GRKKRRQRRR; and (3) RTTI 50, which is identical to the RT003 formulation, except that is does not contain the positively charged carrier present in RT003.

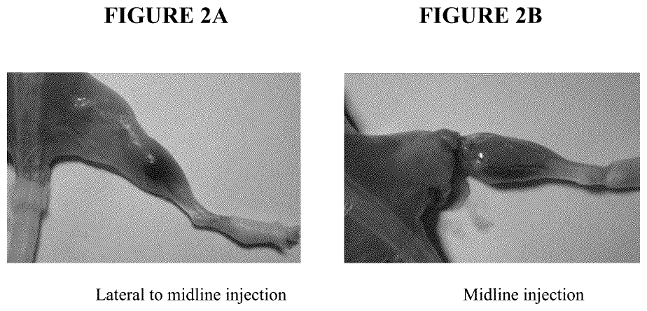

The gastrocnemius muscle of each of the mice used in the study was injected with one of the aforementioned botulinum toxin formulations, either at the lateral-to-midline portion of the muscle (FIG. 2A), or at the midline portion of the muscle (FIG. 2B). DAS assays were performed on each of the mice for four days after injection with the botulinum toxin to determine whether the botulinum toxin of the respective formulation exhibited any tendency to diffuse from the gastrocnemius muscle toward the hind paws of the mouse. From the DAS assays, any decreased ability of the test animals to abduct their hind digits was interpreted as an indication of botulinum toxin diffusion.

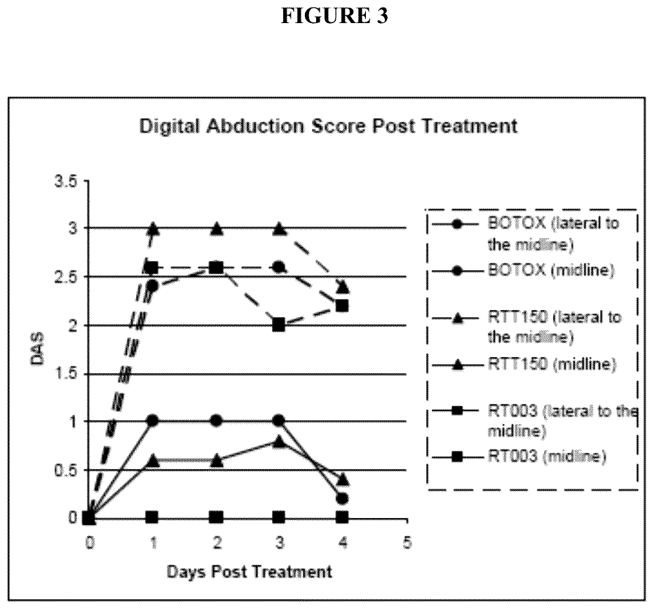

FIG. 3 shows the results of the DAS assays performed after injecting the test animals with the different botulinum toxin formulations as described above. Note that the digital abduction scores are grouped into two clusters, corresponding to whether the injection was at the midline or the lateral-to-midline portion of the gastrocnemius muscle. The generally lower DAS scores for midline injections, as compared to DAS scores for lateral-to-midline injections, indicates that the degree of paralysis in the hind paws of the test animals is generally less following midline injection. Without wishing to be limited by theory, it is believed that this behavior results from the greater distance that botulinum toxin has to travel to reach the hind digits of a test animal following midline injection, as compared to lateral-to-midline injection. This greater required distance of travel by the botulinum toxin is believed to decrease the likelihood of paralysis of the hind digits.

FIG. 3 shows a digital abduction score of zero for all four days following midline injection of the RT003 formulation. This result indicates that the botulinum toxin in the RT003 formulation stays localized in the midline portion of the gastrocnemius muscle upon injection and that no paralysis-causing diffusion occurs on the timescale of the experiment. By contrast, digital abduction scores above the 0.4 DAS baseline are observed following injection of the RTT150 and BOTOX® formulations, with the average DAS score being higher for the BOTOX® formulation. The DAS results for the RTT150 and BOTOX® formulations indicate that hind digit paralysis of the test animals was observed after midline injection of these formulations, with a greater degree of paralysis observed after the injection of the BOTOX® formulation. These data suggest that the botulinum toxin molecules in the RTT150 and BOTOX® formulations are capable of locally diffusing after injection, with a greater degree of local diffusion for the botulinum toxin molecules in the BOTOX® formulation.

FIG. 3 also shows that hind digit paralysis is observed for all test animals following lateral-to-midline injection, irrespective of the specific botulinum toxin formulation. As discussed above, this greater degree of paralysis following lateral-to-midline injection, as compared to midline injection, is believed to relate to a shorter travel distance for the botulinum toxin to the hind paws of the test animals. However, while all three botulinum toxin formulations exhibit paralysis-causing diffusion following lateral-to-midline injection, the degree of paralysis in test animals injected with RT003 is less, on average, than the degree of paralysis observed for the RTT150 and BOTOX® formulations during the timescale of the experiment. Thus, the DAS assay data corresponding to lateral-to-midline injection is qualitatively similar to that for midline injection in that it shows a decreased tendency for local diffusion of botulinum toxin for the RT003 formulation, as compared to RTT150 and BOTOX®.

A comparison of the local diffusion rate following midline injection and lateral-to-midline injection can be made by considering a parameter called the “diffusion index”, which is defined according to Equation (1):

$$diffusionindex = \frac{midline digital abduction score}{lateral - to - midline digital abduction score} \times 100$$

Since digital abduction scores can range from Oto 4, and lateral-to-midline digital abduction scores are expected to be higher than midline digital abduction scores (as discussed above), diffusion index values will typically range from O to 100. A diffusion index value that approaches 100 indicates that the ratio of the midline and lateral-to-midline digital abduction scores approaches unity. This may occur if the rates of diffusion following injection are sufficiently high that the diffusion times for the botulinum toxin to reach and to paralyze the hind digits of the test animal following midline and lateral-to-midline injection are comparable or nearly the same. At the other extreme, diffusion index values that approach zero indicate that the ratio of the midline and lateral-to-midline digital abduction scores is approaching zero. This may occur if diffusion of the botulinum toxin following midline injection is so low that it is insufficient to cause paralysis in the hind digits of the test animals, even though paralysis is observed following lateral-to-midline injection.

Table 1 below shows diffusion index values calculated using digital abduction scores following midline or lateral-to-midline injection of BOTOX®, RT003, and RTT150, as reported in the experiment corresponding to FIG. 3. On the timescale of the experiment, the diffusion index values corresponding to injection of the BOTOX® formulation are higher than the values observed for the RTT150 and RT003 formulations. This indicates that, for injection of the BOTOX® formulation, the ratio of the midline and lateral-to-midline digital abduction scores are closer to unity, compared to the ratios observed for the RTT150 and RT003 formulations. Since botulinum toxin must diffuse further to cause hind-digit paralysis of a test animal following midline injection, the observation that the ratio of the midline and lateral-to-midline digital abduction scores following BOTOX® injection is closer to unity suggests that the botulinum toxin diffusion rate following midline injection of BOTOX® is fairly substantial relative to the rate following lateral-to-midline injection. In other words, the increased diffusion path length associated with midline injection is less of a barrier to causing hind-digit paralysis.

In contrast, the diffusion index values for RT003 are all zero on the four-day timescale of the experiment. This result indicates that no paralysis-inducing diffusion is observed following midline injection of RT003. In other words, the RT003 formulation, which contains the type A botulinum toxin molecule non-covalently associated with a positively charged carrier, permits enhanced localization injected type A botulinum toxin. In this way, the RT003 formulation affords an improved safety profile compared to that of the BOTOX® formulation and minimizes unwanted paralysis.

The observed diffusion index values for RTT150, while not zero as in the case of RT003, are still less than those observed for the BOTOX® formulation. See, Table 1. This result indicates that enough botulinum toxin diffusion occurs to produce observable hind digit paralysis on the four-day timescale of the experiment, but that the time required for paralysis-causing diffusion of botulinum toxin is relatively longer following midline injection.

TABLE 1
Botulinum toxin diffusion index measurements for
RTT150, BOTOX ® and RT003

| | Days Post Treatment | | | | |
|---|---|---|---|---|---|
| | 0 | 1 | 2 | 3 | 4 |
| BOTOX ® | NA | 42 | 38 | 38 | 9 |
| RT003 | NA | 0 | 0 | 0 | 0 |
| RTT150 | NA | 20 | 20 | 27 | 17 |

### (i) Example 3: Injectable Botulinum Toxin Formulations with Reduced Tendency to Generate Antibodies

When botulinum toxin is periodically injected into a patient to treat an unwanted condition such as wrinkles, it is often observed that efficacy of the botulinum toxin decreases with successive injections, even though the duration of the effects of the botulinum toxin may remain the same. This phenomenon is believed to be the result of the formation of antibodies to the botulinum toxin by the immune system of the patient. From a treatment perspective, the formation of antibodies to botulinum toxin by the patient is undesirable, because increasingly larger doses of botulinum toxin are then required to achieve the same effect, which presents serious issues related to both safety and cost.

In certain embodiments, this invention provides injectable botulinum toxin formulations that have a decreased tendency to induce antibody formation, as compared to currently available commercial injectable botulinum toxin formulations. Thus, in these embodiments, botulinum toxin formulations help to minimize the risk associated with botulinum toxin injection by permitting one, over time, to use less toxin to achieve the same effect.

In this example, the DAS assay data obtained after repeated RT003 and BOTOX® injections as described in Example 2 are analyzed as a function of time to determine how the efficacy of these two formulations changes upon repeated administration to the same test animals. Generally, after repeated administration of either formulation, the duration of effects associated with botulinum toxin were the same. However, the degree of muscle paralysis upon repeated administration varied depending on the formulation. To quantify the change in the degree of muscle paralysis, the percent change in the digital abduction scores following injection of either RT003 or BOTOX® was determined according to Equation (2):

$$\% change in DAS = \frac{DAS for nth treatment - DAS for first treatment}{DAS for first treatment} \times 1000$$

Since the numerator of Equation (2) is the difference between the measured digital abduction scores for the n^{th} and the first treatment, the percent change in DAS will be negative if the digital abduction score measured for the n^{th} treatment is less than the digital abduction score measured for the first treatment. In other words, the percent change in DAS is negative when less paralysis is observed after the nth treatment, as compared to the first treatment. Table 2 shows the percent change in the measured DAS values following repeated administration of RT003 and BOTOX® formulations according to the procedure described in Example 2.

TABLE 2
Percent Change in DAS Value after Repeated Administration
of RT003 and BOTOX ®

| | 1^{st} treatment | 1^{st} retreatment | 2^{nd} retreatment |
|---|---|---|---|
| RT003 | 0% | 0% | −30% |
| BOTOX ® | 0% | −44% | −67% |

As indicated in Table 2, after the first retreatment, the percent change in the digital abduction score was −44% for the BOTOX® formulation, which suggests a substantial drop in the efficacy. In contrast, the percent change in the digital abduction score for the RT003 formulation was zero, indicating that the DAS score after the second retreatment was the same as after the initial administration and first retreatment. This result indicates that the degree of paralysis observed after the first retreatment of RT003 is the same as the degree of paralysis following the first treatment and that negligible formation of neutralizing antibodies occurred in the test animals even after the first retreatment. After the 2nd retreatment of RT003 and BOTOX®, the calculated percent changes in DAS values were negative for both formulations, although the magnitude of the percent change in DAS values for the RT003 formulation was half of the value determined for BOTOX®. The larger and negative percent change in DAS values observed for BOTOX® suggest that the test animals had a higher rate of antibody generation to BOTOX®, as compared to RT003. Thus, these data indicate that formulations contemplated by this invention, such as RT003, may have a lower tendency to induce the formation of antibodies that neutralize the effect of botulinum toxin. Accordingly, this result suggests that by using formulations contemplated by this invention, one can, over time, use less botulinum toxin to achieve the same therapeutic effect.

### (i) Example 4: Injectable Botulinum Toxin Formulations With Improved Stability

This example demonstrates that the positively charged carrier molecules used in the injectable botulinum toxin formulations of the invention not only enhance the safety profile of the formulations (Example 2), but also improve their stability. Table 3 shows the results of aging experiments wherein the RT003 and RTT150 formulations are aged at 4° C. (RT003 only) and at 40° C. (both RT003 and RTT150) for various time intervals. After aging at the specified temperatures for the specified times, the potency of the RT003 and RTT150 formulations were measured via a series of mouse IP LDso assays. The results, summarized in Table 3, indicate that the potency of RT003 is essentially unchanged following aging at 4° C., even after six months. Furthermore, the potency of the RT003 formulation, as measured by the formulation's ability to kill the target animals in a mouse IP LDso assay, decreases only slightly even if the RT003 formulation is aged at elevated temperature (40° C.) for six months. By contrast, the RTT150 formulation exhibited a significant decrease in potency following only one month of aging at 40° C. Since the RT003 and RTT150 formulations are identical, with the exception that the RT003 formulation also contains a positively charged carrier molecule having the formula RKKRRQRRRG-(K)15-GRKKRRQRRR, these data indicate that the positively charged carrier molecule improves the stability of the botulinum toxin in the RT003 formulation.

TABLE 3
Results of Mouse IP LD₅₀ Assays following Aging of RT003
and RTT150 At Various Conditions

| | Condition | Time | |
|---|---|---|---|
| | (° C.) | (months) | % Target |
| RT003 | 4 | 0 | 100% |
| | 4 | 6 | 118% |
| | 40 | 6 | 93% |
| RTT150 | 40 | 1 | <50% |

### (i) Example 5: Injectable Botulinum Toxin Formulation Showing Long-Lasting Duration Effects in the Treatment of Glabellar Lines

This Example describes a clinical study and interim analysis of results at week 24 to evaluate the safety, efficacy and duration of effect of an injectable composition of the invention containing botulinum toxin A and a positively charged carrier comprising a positively charged polylysine polypeptide having covalently attached one or more positively charged efficiency groups, called RT002. The RT002 product is an injectable formulation, which contains the 150 kD subtype A botulinum toxin molecule, which is not covalently associated with a positively charged carrier peptide having the formula RKKRRQRRRG-(K)15-GRKKRRQRRR (RTP004; SEQ ID NO: 4), and which does not contain accessory proteins or animal-derived components. RT002 is used in the study for the treatment of moderate to severe glabellar lines. The excipient comprises 0.1 mg polysorbate 20, 36 mg trehalose dihydrate, and 11.7 μg RTP004, per 50 U of the 150 kDa type A toxin without accessory proteins and the treatment dose is 40 U.

The clinical study was a phase 2, randomized, double blind, dose ranging, active and placebo-controlled, multi-center study designed and conducted to evaluate the safety and efficacy and duration of effect of a single (one-time) treatment by injection of RT002 for the temporary improvement in the appearance of glabellar lines in adults. Three doses, 20 U, 40 U and 60 U, of RT002 were evaluated compared to an active, i.e., VISTABEL®/BOTOX® (20 U dose by intramuscular injection) and a placebo control (intramuscular injection). The injection treatment was a single intramuscular injection. The duration of effect of a single treatment of RT002 at the three dosage levels versus VISTABEL®/BOTOX® Cosmetic was also assessed.

The RT002 product is composed of purified 150 kDa botulinum neurotoxin without accessory proteins, referred to as RTT150, formulated in a lyophilized powder. In nonclinical studies, RT002 has been shown to exhibit less diffusion than other forms of botulinum neurotoxin A (BoNTA) and may offer more control of effect at target sites with less side effects due to distant spread of toxin into neighboring muscles. In addition, the RT002 additive-free botulinum toxin type A formulation has the ability to afford less immunogenic potential due to the absence of non-active proteins present in the formulation. In addition, RT002 was well tolerated after repeat dose intramuscular administration of up to 50 U/kg in rats.

Dosing regimen and injection technique: The dosing regimen of RT002 for this study was a single treatment of either RT002 (20 U, 40 U, or 60 U), placebo, or VISTABEL®/BOTOX®, which was dosed at 20 U per subject, as a 0.1 mL intramuscular injection into each of 5 injections sites on the forehead (0.5 mL total), between the eyebrows, of the subject undergoing treatment. All treatments were intramuscular injections administered by a trained physician. More specifically, study subjects received a single treatment of 0.1 mL per injection treatment to five injection sites: two injections into each corrugator muscle, and one injection in the procerus muscle. Investigators, site staff, subjects, and the sponsor were blinded to the treatment group assignments. Approximately 250 adult, female and male subjects, 30 to 65 years of age and in good general health, with moderate to severe glabellar lines at entry, were enrolled in the study.

Glabellar facial lines arise from the lateral corrugator and vertical procerus muscles in the face. These can be readily identified by palpation of the muscle mass while having the patient frown maximally. The corrugator depresses the skin creating a vertical line, i.e., a furrow, surrounded by ridges of tensed muscle (i.e., frown lines). Because the location, size and use of the muscles vary markedly among individuals, physicians administering injectable botulinum toxin must understand the relevant anatomy of the area involved and any alterations to the anatomy due to prior surgical procedures. In order to reduce the risk of ptosis, the following steps are optimally performed: (i) injection of or near the levator palpebrae superioris should be avoided, particularly in patients with larger brow depressors; (ii) medial corrugator injections should be at least 1 centimeter above the bony supraorbital ridge; (iii) it should be ensured that the injected volume/dose is accurate; and (iv) toxin should not be injected closer than 1 centimeter above the central eyebrow. Botulinum toxin is injected by applying finger pressure on the superior medial orbital rim while advancing the needle through the skin into the underlying muscle.

For the study, the severity of a subject's glabellar lines was assessed by the Investigator and the subject. For the Investigator assessment, an Investigator Global Assessment-Facial Wrinkle Severity (IGA-FWS) rating score system was used as follows: an IGA-FWS rating score of (0) indicated no facial wrinkle severity; an IGA-FWS rating score of (1) indicated mild facial wrinkle severity; an IGA-FWS rating score of (2) indicated moderate facial wrinkle severity; and an IGA-FWS rating score of (3) indicated severe facial wrinkle severity. As appreciated by the skilled practitioner, a photo guide exhibits the grades of wrinkle severity used for Investigator and reference.

Patient Facial Wrinkle Severity (PFWS) Assessment: A Patient Facial Wrinkle Severity (PFWS) was used for a subject's assessment of his/her facial wrinkle severity. Subjects completed the Patient Facial Wrinkle Severity (PFWS) at maximum frown to assess the severity of the glabellar lines at the Screening Visit, Treatment Visit (Day 0) pre-treatment, Follow-up Visits (Weeks 2, 4, 8, 12, 16, 20, 24, 28, 32), and End-of-Study Visit (Week 24, 28, 32 or 36, as appropriate) or Early Discontinuation Visit, if applicable. The assessment form was provided directly to the subject to complete while reviewing the glabellar lines using a supplied handheld mirror. The PFWS rating score system was as follows: a PFWS rating score of (0) indicated no wrinkle severity, with associated description of “no wrinkles;” a PFWS rating score of (1) indicated mild wrinkle severity, with associated description of “very shallow wrinkles;” a PFWS rating score of (2) indicated moderate wrinkle severity, with associated description of “moderate wrinkles;” and a PFWS rating score of (3) indicated severe wrinkle severity, with associated description of “deep wrinkles.” In accordance with the study, an IGA-FWS and a PFWS rating of (2) moderate or (3) severe for a subject's glabellar lines were required for a subject to be enrolled in the study.

Subjects were randomized 1:1:1:1:1 to one of the treatments of Table 4 below.

TABLE 4
Description of Treatment Groups

| Treatment Group | Test Article and Dose | No. of Subjects |
|---|---|---|
| 1 | RT002 20 U | 50 |
| 2 | RT002 40 U | 50 |
| 3 | RT002 60 U | 50 |
| 4 | Placebo | 50 |
| 5 | Active comparator | 50 |
| | (VISTABEL ®/BOTOX ® 20 U) | |

Subjects enrolled in the study had screening and treatments visits and follow-up safety and efficacy evaluations throughout the study for up to 36 weeks. A subject diary was provided for the initial 2-week period to document onset of treatment response. Subjects were evaluated with a phone call at Week 1 and during visits at Weeks 2, 4, 8, 12, 16, 20, 24, 28, 32 and 36 of the study. All subjects were followed for at least 24 weeks post-treatment. If the subject's Investigator Global Assessment-Facial Wrinkle Severity (IGA-FWS) score at maximum frown returned to baseline between the 24 week and 36 week visits, the visit at which that score was recorded was considered the End-of-Study Visit for the subject.

The study duration was up to 38 weeks of study, including a screening period of up to two weeks followed by a single treatment, and a follow-up period of up to 36 weeks post-treatment. All subjects were followed for at least 24 weeks post-treatment. Injection sites were evaluated at the Screening Visit, Treatment Visit (Day 0), pre- and post-treatment (to determine if there was an immediate reaction to the investigational product), Follow-up Visits (Weeks 2, 4, 8, 12, 16, 20, 24, 28, 32), and End-of-Study Visit (Week 24, 28, 32 or 36, as appropriate) or Early Discontinuation Visit, if applicable. The assessment was done as a global evaluation of the 5 injection sites and evaluated erythema, edema, burning or stinging sensation, and itching, as described by the subject.

In addition, cranial nerves II-VII were evaluated by the Investigator at the Treatment Visit (Day 0) pre-treatment, at Follow-up Visits (Weeks 2, 4, 8, 12, 16, 20, 24, 28, 32) and at the End-of-Study Visit (Week 24, 28, 32, or 36, as appropriate) or Early Discontinuation Visit, if applicable. Scores for the cranial nerve assessments were captured as follows: a rating of (1) corresponded to “Normal”; a rating of (2) corresponded to “Abnormal, not clinically significant;” a rating of (3) corresponded to “Abnormal, clinically significant;” a rating of (4) corresponded to “Not assessed.” For these assessments, cranial nerve II is the optic nerve; cranial nerve III is the oculomotor nerve; cranial nerve IV is the trochlear nerve; cranial nerve Vis the trigeminal nerve; cranial nerve VI is the abducens nerve and cranial nerve VII is the facial nerve. The Regional House-Brackmann Facial Nerve Grading System (Yen, T L. et al., 2003, Oto!. Neurotol., 24(1):118-122) was designed to evaluate synkinesis and the four major branches of the facial nerve (VII) that innervates target and adjacent musculature. The Investigator evaluated functionality of the facial nerve (VII) at the Treatment Visit (Day 0) pre-treatment, Follow-up Visits (Weeks 2, 4, 8, 12, 16, 20, 24, 28, 32) and End-of-Study Visit (Week 24, 28, 32, or 36, as appropriate) or Early Discontinuation Visit, if applicable.

Facial muscle strength was evaluated using the Medical Research Council Scale for Assessment of Muscle Power (MRC). The MRC is a reliable and validated scale for assessing muscle weakness and aids the investigation of peripheral nerve injuries (Paternostro-Sluga, 2008). The orbicularis oculi (eyelid), lateral brow elevators, and lateral orbicularis zygomaticus muscles on each side of the face were evaluated. In the MRC Scale for Muscle Power Assessment, a rating of (0) corresponds to “no movement;” a rating of (1) corresponds to “flicker perceptible in the muscle;” a rating of (2) corresponds to “movement only is gravity is eliminated;” a rating of (3) corresponds to “can move limb against gravity;” a rating of (4) corresponds to “can move against gravity and some resistance exerted by examiner; and a rating of (5) corresponds to “normal power.”

Distant spread of toxin queries were conducted with subjects at the Treatment Visit (Day 0) post-treatment, Follow-up Phone Call (Week 1), Follow-up Visits (Weeks 2, 4, 8, 12, 16, 20, 24, 28, 32), and End-of-Study Visit (Week 24, 28, 32, or 36, as appropriate) or Early Discontinuation Visit, if applicable. In addition, adverse events (AEs) were also evaluated at these same time points. Without wishing to be limiting, examples of AEs include double vision, eyelid paralysis, muscle weakness, extreme tiredness and difficulty swallowing, breathing and speaking.

Efficacy assessments included Investigator assessment of glabellar line severity and glabellar line improvement scales, subject assessment of glabellar line severity and improvement including subject questionnaires, and onset of effect evaluated by subject diary. Efficacy assessments were conducted with the subject in a sitting position. In order to have consistent eye positioning during the assessment, the Investigator asks the subject to focus on a fixed point in the examination room. The assessment should be conducted in a room with good overhead lighting (an exam light should not be used) or natural light from a window (but not direct sunlight). At each clinic visit, the visual appearance (at maximum frown and at rest after maximum frown) of the glabellar lines was assessed by the Investigator using a fit-for-purpose 4 point IGA-FWS scale/rating score for Facial Wrinkle Severity Score, as follows: a rating score of (0) corresponded to no facial wrinkles; a rating score of (1) corresponded to mild facial wrinkles; a rating score of (2) corresponded to moderate facial wrinkles; and a rating score of (3) corresponded to severe facial wrinkles. The assessment represented wrinkle severity at each given time-point and was not based on a comparison to the pre-treatment level. Assessments were optimally completed by the same Investigator and as close as possible to the same time of day at each visit. In an effort to standardize the rating of wrinkle severity across Investigators, a set of training photographs exhibiting the grades of wrinkle severity was used for Investigator training. A photo guide was also provided to each study center to assist in the Investigator's assessment.

Patient Global Aesthetic Improvement Scale (GAIS): The Investigator and subject assessed the visual appearance (at maximum frown and at rest after maximum frown) of the glabellar line improvement from the baseline condition using the 7 point severity Patient Global Aesthetic Improvement Scale (GAIS) shown in Table 5 below. Study subjects completed the Patient Global Aesthetic Improvement Scale (GAIS) at maximum frown and at rest after maximum frown, to assess the visual appearance of the glabellar line improvement from the baseline condition at Follow-up Visits (Weeks 2, 4, 8, 12, 16, 20, 24, 28, 32), and End-of-Study Visit (Week 24, 28, 32, or 36, as appropriate) or Early Discontinuation Visit, if applicable. The GAIS assessment form was provided directly to the subject to complete while reviewing the treated area using a supplied handheld mirror. Subjects with contact lenses optimally viewed their glabellar lines while wearing their contacts. Subjects wearing glasses were advised to view their glabellar lines without glasses if possible. If glasses were needed for the subject to see their glabellar lines, then glasses were worn for the assessment. The subject assessment was completed before the Investigator completed the IGA-FWS assessment.

TABLE 5
Global Aesthetic Improvement Scale

| Rating Score | Wrinkle Improvement |
|---|---|
| −3 | Very Much Worse |
| −2 | Much Worse |
| −1 | Worse |
| 0 | No Change |
| 1 | Improved |
| 2 | Much Improved |
| 3 | Very Much Improved |

At each clinic visit, the subject assessed the visual appearance (at maximum frown) of the glabellar lines using the following fit-for-purpose 4 point scale for subject's assessment of Patient-Facial Wrinkle Severity (Table 6 below). The assessment form was provided directly to the subject to complete while reviewing the glabellar treatment area using the supplied handheld mirror. As for the above GAIS assessment, subjects with contact lenses optimally viewed their glabellar lines while wearing their contacts. Subjects wearing glasses were advised to view their glabellar lines without glasses if possible. If glasses were needed for the subject to see their glabellar lines, then glasses were worn for the assessment. The subject assessment was completed before the Investigator completes the IGA-FWS assessment. The assessment represented wrinkle severity at each given time-point and was not based on a comparison to the pre-treatment defect level. Assessments were optimally completed by the subject as close to the same time as possible at each visit.

TABLE 6
Patient-Facial Wrinkle Severity (PFWS)

| Rating | Wrinkle | Description |
|---|---|---|
| 0 | None | No wrinkles |
| 1 | Mild | Very shallow wrinkles |
| 2 | Moderate | Moderate wrinkles |
| 3 | Severe | Deep wrinkles |

Additional subject assessments during the study included a rating of the importance of the duration of effect when choosing an aesthetic treatment (provided at the Treatment Visit (Day 0); a rating of subjects' satisfaction with the treatment results (at the Week 4 visit), in the form of a questionnaire to rate their satisfaction with the treatment results—the subjects were asked how satisfied or dissatisfied they were with the appearance of the treated area of the face; and a rating of their satisfaction with the duration of the treatment effect (at the End-of-Study Visit (Week 24, 28, 32, or 36, as appropriate) or Early Discontinuation Visit, if applicable.

Digital photographs of the treatment area were taken at the Treatment Visit (Day 0) pre-treatment, Follow-up Visits (Weeks 2, 4, 8, 12, 16, 20, 24, 28, 32), and at End-of-Study Visit (Week 24, 28, 32, or 36, as appropriate) or Early Discontinuation Visit. Digital photographs were taken in a controlled and standardized manner. Reference photographs and appropriate training were provided to site staff and Investigators. Subjects optimally did not wear eye or facial make-up of any kind. In order to minimize light reflection from the skin, treated areas were blotted with an alcohol pad and allowed to dry to remove skin oil prior to taking any photographs. Photographs included the subject's frontal view at maximum frown and at rest after maximum frown.

Statistical Analysis: All statistical programming and analyses were performed using SAS version 9.3 or higher. As this study was not powered to detect any statistically significant differences between treatment groups at the 0.05 level, the p-value obtained from various tests described below are expected to establish statistical trending. No adjustments were made for multiplicity of testing. Demographic and baseline characteristics were summarized for the intent-to-treat (ITT), per-protocol (PP) and safety populations. Descriptive statistics were provided for all efficacy variables at all time-points by treatment group as well as by treatment group and geography/country. Efficacy analyses were performed for the ITT and PP populations. Safety analyses were performed on the safety population.

Populations: All subjects who were randomized and received treatment (at least I dose of study medication) were included in the intent-to-treat (ITT) population. All subjects who were randomized, received treatment, and had provided at least one post-treatment safety assessment were included in the Safety Population. The Per Protocol (PP) population included subjects from the ITT population who complete the 24-week evaluation without a major protocol violation. Subjects were excluded from the PP population for any of the following reasons: (i) the subject violated inclusion/exclusion criteria; (ii) the subject missed the week 24 visit; (iii) the subject used a prohibited medication; (iv) the subject's Week 24 visit was ±5 days off-schedule (outside of allowed variation in scheduled visit days).

For safety groups and efficacy comparisons, subjects were randomized into 5 treatment groups (RT002 20 U; RT002 40 U; RT002 60 U; Placebo; Active Comparator). The primary efficacy comparisons were performed between each RT002 dose and active comparator; each RT002 dose and placebo; as well as active comparator to placebo. A risk-to-benefit ratio was evaluated to examine trends in favor of at least one of the RT002 doses versus active comparator in key study evaluations (proportion of responders at month 6 and duration of response measured up to 36 weeks; frequency of AEs).

Within each treatment group, the missing scores for IGA-FWS, PFWS, and GAIS for the ITT population were imputed by Markov Chain Monte Carlo (MCMC) multiple imputation for analyses based on proportion of responders. The sensitivity analysis for the primary endpoint was performed using imputations based on the last observation carried forward method.

Descriptive statistics were used to summarize demographic characteristics (e.g., age, gender, race, etc.) and background characteristics (e.g., IGA-FWS, PFWS, etc.). Past or ongoing medical history, study visit compliance, and prior and concomitant medication usage were summarized for all subjects and presented in a listing by subject.

Efficacy: For efficacy, primary clinical efficacy were assessed by blinded evaluator who graded the severity of the subject's glabellar lines at maximum frown using the IGA-FWS. A responder is defined as a subject who has a one point or greater improvement in IGA-FWS versus baseline and who has not returned to baseline IGA-FWS at the time point of evaluation. For the primary analysis purposes, the Proportion of Responders was compared between each RT002 dose and active comparator at Week 24. Each RT002 treatment group was compared separately to placebo and active comparator. Active comparator was compared to placebo at each visit also. Comparisons were made with Cochran-Mantel-Haenszel (CMH) tests stratified by baseline IGA-FWS.

For the primary analysis purposes, Duration of Response was compared between each of the RT002 doses and active comparator using the Kaplan-Meier method. The duration of response was measured from the time of injection to the time point when a subject reverted to his/her baseline severity as measured by Blinded Investigator based on IGA-FWS. If the subject did not achieve a one point improvement from baseline by IGA-FWS on or before Week 4, the duration of response was considered zero. If subject achieved at least a I point improvement based on IGA-FWS on or before week 4, but did not revert to his/her baseline by Week 36 (last time point), such a subject was censored at Week 36 (date of last evaluation) for the analysis. The log-rank test was used to compare duration of response between RT002 and active comparator. A Risk to Benefit Ratio computed for each treatment group was equal to the sum of the number of treatment related adverse events divided by the sum of the duration of response days for the subjects in the treatment group. If a subject achieved at least 1 point improvement based on IGA-FWS on or before week 4 but did revert to his/her baseline by Week 36 (last time point), then his/her contribution to the benefit sum was the number of days between baseline and the last visit day.

For secondary analyses, secondary endpoints used are defined as follows: (1) Proportion of Responders at Week 2 and at Weeks 4-36 with the emphasis on Week 12 and Week 24 evaluations. The comparisons between treatment groups were based on the CMH test stratified by baseline severity of the variable analyzed where possible. Each treatment group was compared to placebo and separately compared to active comparator for those subjects who had a baseline severity which could possibly permit the required improvement for success. Active comparator was also compared to placebo at each visit.

Responders were evaluated based on several definitions: (i) those who improve by at least 2 points based on IGA-FWS versus Baseline; (ii) those who improve by at least 1 point based on IGA-FWS versus Baseline; (iii) those who have IGA-FWS scores of O or 1; (iv) those who improve by at least 1 point based on PFWS; (v) those who improve by at least 2 points based on PFWS; (vi) those who have a score of at least 1 on GAIS scale;

(2) Secondary endpoints based on various definitions of duration of response. Subjects who did not achieve an improvement as specified in each definition below by Week 4 were assigned O duration; subjects who achieved an improvement as defined below but did not revert back to baseline by Week 36 were censored at Week 36 (date of last evaluation). Treatment groups were compared using the log rank test. For each definition and treatment group, a risk to benefit ratio was computed as described above. Definitions for duration of response include (i) time from injection to GAIS score less than 1 for a responder definition of at least 1 in GAIS; (ii) time from injection to reversion to baseline for a responder definition of at least 2 point improvement in IGA-FWS; (iii) time from injection to reversion to baseline for a responder definition of at least 1 point improvement in PFWS; (iv) time from injection to reversion to baseline for a responder definition of at least 2 point improvement in PFWS; (v) time from injection to reversion to baseline for a responder definition of at least 1 point improvement in IGA-FWS using proportional hazards model with term for treatment, baseline severity, and treatment by baseline severity interaction; and (vi) time from injection to reversion to baseline for a responder definition of at least I point improvement in PFWS using proportional hazards model with term for treatment, baseline severity, and treatment by baseline severity interaction. An exploratory analysis was conducted to correlate the subject's GAIS assessment scores with the responder rates based on the PFWS for both 1- and 2-point changes. Correlation analyses and logistic regressions were used, as appropriate.

Patient Data: Patient reported outcomes supported investigator findings of duration and efficacy of RT002 treatment. At 24 Weeks (6 months), the 40 U RT002 dose continued to deliver clinically meaningful higher response rates on the Subject Global Aesthetic Improvement Scale (GAIS) with 46.3% of the RT002 40 U-treated subjects versus 31% of BOTOX® Cosmetic-treated subjects having a rating score of at least a 1. At week 16, compared with the “up to 120 days” duration of BOTOX® Cosmetic, based on its label information, RT002 40 U dose achieved statistically significant higher response rates as measured by at least a I-point improvement on the Patient Wrinkle Severity (PWS) Scale and at least a I-point rating on the Subject Global Aesthetic Improvement Scale. 76.9% of subjects treated with RT002 40 U maintained at least a I-point improvement on PWS compared with 58.5% of subjects treated with BOTOX® Cosmetic. In addition, 89.7% of subjects treated with RT002 40 U maintained at least a I-point score on GAIS compared with 70.7% of subjects treated with BOTOX® Cosmetic.

Safety: The RT002 product exhibited a safety and efficacy profile highly comparable to BOTOX® Cosmetic. Adverse events were generally mild, and were mainly associated with effects from the injection itself All RT002 dose groups exhibited an excellent overall safety profile with AEs that were predominantly localized, transient and mild in severity. No serious AEs occurred in any active dose group. The 20 U and 40 U RT002 dose groups were well tolerated and clinically superior to BOTOX® with respect to causing Ptosis. In addition, RT002 exhibited less downward spread at the 20 U an 40 U doses. Both the 20 U and the 40 U doses cause No Ptosis in any subject treated with those doses of RT002 at any time point, compared to 1.9% in the BOTOX® Cosmetic treated group. A 5.7% ptosis rate was observed in subjects of the RT002 60 U treatment group. These were transient in nature, as typically seen with BOTOX® treatment. The reduced diffusion of RT002 is consistent with nonclinical and prior studies and supports a reduced spread of toxin, as observed in subjects treated with compositions of the invention which contain botulinum toxin, such as botulinum toxin A, and a positively charged carrier comprising a backbone, such as polylysine, with one or more covalently attached, positively charged efficiency groups as described herein, such as RT002.

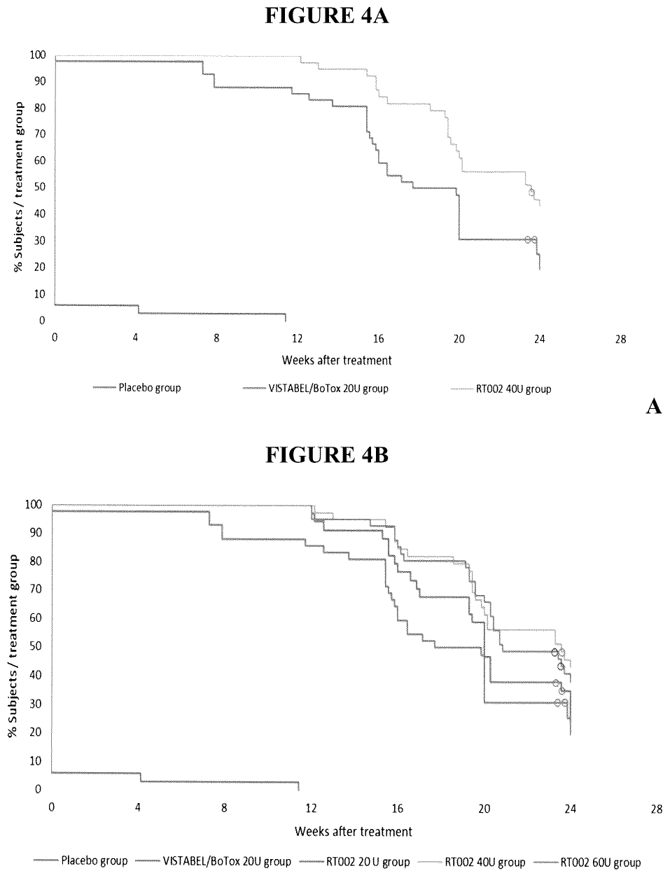

Dosage and Duration of Effect: Without wishing to be limiting, the interim analysis results support a dose selection of 40 U as an optimal dose for single treatment with the botulinum containing compositions of the invention, based on the high responder rates, duration of effect and positive safety profile. In addition, the compositions of the invention, such as RT002, have a sustained and long lasting duration of effect, e.g., for at least 6 months, following administration by injection to a subject. As determined from the interim analysis of the study results, treatment of subjects' glabellar lines with the RT002 product achieved a superior duration effect when compared to treatment of glabellar lines in subjects with the BOTOX® Cosmetic. Indeed, a 5.9-month median duration of I-point improvement on IGA in the RT002 40 U dose group (23.6 weeks) was demonstrated versus an 18.8 week duration in subjects treated with BOTOX® Cosmetic (p=0.020) based on Kaplan Meier analysis method. (See, e.g., FIGS. 4A and 4B). Of note, at month 6, a significant number of RT002-treated subjects were censored from the interim analysis of duration since they were still responders. At month 6, nearly one third (−33%) of the subjects in the RT002 40 U treatment group still had no, or almost no, wrinkles after a single treatment (p=0.041) versus 12% of subjects in the BOTOX® Cosmetic treatment group. Further, the high dose group was followed for 32 weeks post-treatment to assess duration of response and achieved a median duration of 29.4 weeks or 7.3 months based on both investigator and subject assessments.

The duration of effect provided by compositions of the invention, such as RT002, as well as treatment methods and uses, thereof afford advantages that subjects undergoing treatment consider to be of high importance to them for an aesthetic treatment. Such a long, sustained duration of effect, particularly achieved by a single or one-time injection dose of product, namely, RT002, permits fewer injections per treatment course for a subject, which is important for the subject's comfort, convenience and overall well-being. A product that affords significant and sustained effects, maintained for at least a 6-month period following a single treatment dose by injection of the product to a subject, provides a solution to an unmet need in the art for both practitioners and patients.

Summary of interim results: The results demonstrate that a composition of the invention as represented by the RT002 product proved superior to BOTOX® Cosmetic as measured by median duration of effect and responder rates at I-point and 2-point improvement on IGA-FWS, and percentage of patients who achieved and maintained no wrinkles or mild wrinkles pursuant to the IGA-FWS scoring system described above. The study achieved statistically significant results for the primary efficacy endpoint of I-point improvement on IGA-FWS at 28 days. The week 24 interim analysis demonstrated clinically meaningful differentiation in results afforded by single treatment of subjects with an injected dose of RT002 versus injection with BOTOX® Cosmetic.

As also determined by the interim analysis, RT002 achieved an approximately 6-month duration of effect with high responder rates. RT002 achieved superior duration of effect compared with BOTOX® Cosmetic, demonstrating a 5.9-month median duration of I-point improvement in glabellar lines based on the Investigator Global Assessment-Facial Wrinkle Severity (IGA-FWS) scale in the 40 U dose group (23.6 weeks) versus 18.8 weeks for BOTOX® Cosmetic (p=0.020) based on Kaplan Meier analysis method. At 24 weeks (6 months), RT002 at doses of 40 U and 60 U continued to deliver clinically meaningful higher response rates with 35.9% and 29.3% of subjects, respectively, maintaining a I-point improvement versus 19% of BOTOX® Cosmetic-treated subjects. RT002 achieved its primary efficacy endpoint of at least I-point improvement on the investigator scale (IGA-FWS) at 28 days, as well as the patient reported outcome. RT002 achieved 100% response rates in all dose groups at the 28-day primary efficacy endpoint of a I-point improvement on the Investigator Global Assessment Facial Wrinkle Severity Scale (IGA-FWS). RT002 achieved greater than 97% response rates in all dose treatment groups at the 28-day primary efficacy endpoint of a I-point improvement on the Patient Facial Wrinkle Scale. Efficacy data showed 96% of subjects were rated with None or Mild wrinkle severity at maximum frown 4 weeks post-treatment by the clinical investigator assessment and 83% of subjects assessed themselves as achieving None or Mild wrinkles at maximum frown at the same time point. RT002 was well tolerated and no serious adverse events were found. No eyelid Ptosis occurred in subjects in the RT002 20 U or 40 U dose treatment groups. Dose response was observed in the study; subjects who were administered the 40 U dose of RT002 showed particularly high response rates.

Overall, RT002 for Injection has been well tolerated at all dose levels without any systemic or local safety concerns or evidence of spread. RTT150 for Injection has been well tolerated in clinical trials with no evidence of spread beyond the treatment site at any dose. Adverse events in the phase 1/2 dose-escalating, open label clinical trial, RT002-CL001, were generally mild, localized and transient. The most common adverse events observed were headache and injection site reactions. No subject in any cohort experienced ptosis. There were no serious adverse events and adverse event rates did not change in frequency, severity, or type with increasing doses. Thirty-four (34) subjects reported 131 AEs. The most common adverse events reported were headache (31 reports; 17 subjects); injection site pruritus (34 events; 8 subjects), injection site pain (burning) (14 events; 6 subjects), and eye disorders (14 events; 5 subjects). In addition to adverse events, safety evaluations in the RT002-CL001 study included clinical laboratory tests (hematology, chemistry, urinalysis, and prothrombin time), serum antibodies for RTT150 toxin and RTP004 peptide, assessment of cranial nerves II-VII and facial muscle strength, concomitant therapy medication and urine pregnancy test for women of childbearing potential. There was no evidence of spread beyond the treatment site at any dose and no evidence of any systemic exposure based on clinical laboratory results and physical assessments. All subjects were negative for antibodies to both toxin and peptide.

### (i) Example 6: Follow Up Study Regarding Injectable Botulinum Toxin Formulation Showing Long-Lasting Duration Effects in the Treatment of Glabellar Lines

RT002 also was evaluated in a phase 2, dose-ranging, active and placebo controlled clinical trial, RT002-CL002, in Canada, to evaluate the safety, efficacy and duration of a single administration for the treatment of moderate to severe glabellar lines in adults. The trial enrolled 268 subjects (over 50 per treatment group), who were treated with 20, 40 or 60 U of RT002, 20 U of BOTOX Cosmetic, or placebo. For the treatment of glabellar lines, the proposed dosing regimen in the clinical trial was a single treatment of 20, 40 or 60 Units per subject, 0.1 mL intramuscular injection into each of 5 injections sites on the forehead. Doses of 16, 32, 48, or 64 U based on current saline potency method (corresponding to 25, 50, 75 and 100 U in previous gelatin phosphate buffer potency method) were well tolerated in a phase 1/2 clinical trial (Study RT002-CL001; 12 subjects per dose group; 48 subjects total).

The interim data showed that RT002 achieved its primary efficacy measurement for all three doses at 4 weeks. The study demonstrated 6-month RT002 median duration of effect based upon at least I-point improvement in glabellar lines at maximum frown on the Investigator Global Assessment-Facial Wrinkle Severity scale. Subject-reported outcomes were consistent with investigator findings of duration and efficacy of RT002. Across all cohorts, RT002 appeared to be generally safe and well-tolerated. Adverse events were generally mild, localized and transient. There were no serious adverse events or evidence of any systemic exposure at any of the three doses evaluated.

### (i) Example 7: Efficacy and Safety of an Injectable Botulinum Toxin Formulation Showing Higher Responder Rate and Long-Lasting Duration Effects in the Treatment of Moderate to Severe Glabellar Lines (Phase 3 Study, Arm 1 and Arm 2)

This Example describes two arms of a clinical study and primary outcome analysis of results at week 36 to evaluate the safety, efficacy, and duration of effect of an injectable composition of the invention containing botulinum toxin A and a positively charged carrier comprising a positively charged polylysine polypeptide having covalently attached positively charged efficiency groups, called RT002. The RT002 product is an injectable formulation, which contains the 150 kD subtype A botulinum toxin molecule without accessory proteins, which is non-covalently associated with a positively charged carrier peptide having the formula RKKRRQRRRG-(K)15-GRKKRRQRRR (RTP004; SEQ ID NO: 4) and which does not contain accessory proteins or animal-derived components. RT002 is used in the study for the treatment of moderate to severe glabellar lines. The excipient comprises 0.1 mg polysorbate 20, 36 mg trehalose dihydrate, and 11.7 μg RTP004, per 50 U of the 150 kDa type A toxin without accessory proteins (and the treatment dose is 40 U).

Two active treatment arms with RT002 were included in the clinical study, which was a phase 3, randomized, double-blind, placebo-controlled, pivotal, multi-center study designed and conducted to evaluate the safety, efficacy, and duration of effect of a single (one-time) administration by injection of RT002 for the temporary improvement in the appearance of moderate to severe glabellar lines in adults. The dose of 40 U of RT002 was evaluated compared to a placebo control (intramuscular injection). The injection treatment was a single intramuscular injection, placed in 5 different areas within the glabellar complex, one in the procecrus, and one in the medial and lateral aspects of the right and left corrugator muscles (8U per each injection site). The duration of effect of a single treatment of RT002 at the 40 U dosage was also assessed.

The RT002 product is composed of purified 150 kDa botulinum neurotoxin without accessory proteins, referred to as RTT150, formulated as lyophilized powder. In nonclinical studies, RT002 has been shown to exhibit less diffusion than other forms of botulinum neurotoxin A (BoNTA) and may offer more control of effect at target sites with less side effects due to distant spread of toxin into neighboring muscles. In addition, the RT002 additive-free botulinum toxin type A formulation has the ability to afford less immunogenic potential due to the absence of non-active proteins present in the formulation. In addition, RT002 was well tolerated after repeat dose intramuscular administration of up to 50 U/kg in rats. RTP004 has been dosed at maximum feasible dose without effect in dermal, genotoxicity, and reproductive studies and produced no significant findings in parenteral studies at a safety multiple of more than 9,500-fold.

Dosing regimen and injection technique: The dosing regimen of RT002 for this study was a single treatment of either RT002 (40 U) or placebo, as a 0.1 mL intramuscular injection into each of 5 injections sites on the forehead (0.5 mL total), between the eyebrows, of the subject undergoing treatment. All treatments were intramuscular injections administered by a trained physician. More specifically, study subjects received a single treatment of 0.1 mL per injection treatment to five injection sites: two injections into each corrugator muscle, and one injection in the procerus muscle. Investigators, site staff, subjects, and the sponsor were blinded to the treatment group assignments. Approximately 300 adult, female and male subjects, 18 to 75 years of age and in good general health, with moderate to severe glabellar lines at entry, were enrolled in each of the two studies, for a total of about 600 subjects. Specifically, there were 303 patients in the first arm and 306 in the second.

The duration was up to 38 weeks of trial, including a screening period of up to two weeks followed by a single treatment and a follow-up period of up to 36 weeks post-treatment. All patients were followed for at least 24 weeks post-treatment. Starting at Week 24 post-treatment, patients were followed until their wrinkle severity in the glabellar lines at maximum frown returned to baseline in both the Investigator Global Assessment-Facial Wrinkle Severity (IGA-FWS) and Patient Facial Wrinkle Severity (PFWS) assessments.

Glabellar facial lines arise from the lateral corrugator and vertical procerus muscles in the face. These can be readily identified by palpation of the muscle mass while having the patient frown maximally. The corrugator depresses the skin creating a vertical line, i.e., a furrow, surrounded by ridges of tensed muscle (i.e., frown lines). Because the location, size and use of the muscles vary markedly among individuals, physicians administering injectable botulinum toxin must understand the relevant anatomy of the area involved and any alterations to the anatomy due to prior surgical procedures. In order to reduce the risk of ptosis, the following steps are optimally performed: (i) injection of or near the levator palpebrae superioris should be avoided, particularly in patients with larger brow depressors; (ii) medial corrugator injections should be at least 1 cm above the bony supraorbital ridge; (iii) it should be ensured that the injected volume/dose is accurate; and (iv) toxin should not be injected closer than 1 cm above the central eyebrow. Botulinum toxin is injected by applying finger pressure on the superior medial orbital rim while advancing the needle through the skin into the underlying muscle.

### (a) Determination of Sample Size

Estimates of treatment efficacy taken from previous work showed that a sample size of 200 and 100 for RT002 40 U for injection and placebo, respectively, has over 99% power to detect a difference between treatment groups for the primary efficacy endpoint: proportion of 2-point composite responders at Week 4 based on a 2-sided chi-squared test at an alpha level of 0.05 (response rate of at least 50% vs 1%). The sample size of 300 patients was chosen to ensure adequate power to detect a difference between treatment groups for the response rate of key secondary endpoints on later visits.

### (a) Assessment of Glabellar Line Severity

For the study, the severity of a subject's glabellar lines was assessed by the Investigator and the subject. For the Investigator assessment, an Investigator Global Assessment-Facial Wrinkle Severity (IGA-FWS) rating score system was used as follows: an IGA-FWS rating score of (0) indicated no facial wrinkle severity (“None”), described as “no wrinkles;” an IGA-FWS rating score of (1) indicated mild facial wrinkle severity (“Mild”), described as “very shallow wrinkles;” an IGA-FWS rating score of (2) indicated moderate facial wrinkle severity (“Moderate”), described as “moderate wrinkles;” and an IGA-FWS rating score of (3) indicated severe facial wrinkle severity (“Severe”), described as “deep and furrowed wrinkles.” Assessment is made at maximum frown and at rest afterwards. As appreciated by the skilled practitioner, a photo guide exhibits the grades of wrinkle severity used for reference.

Patient Facial Wrinkle Severity (PFWS) Assessment: A Patient Facial Wrinkle Severity (PFWS) was used for a subject's assessment of his/her facial wrinkle severity. Subjects completed the Patient Facial Wrinkle Severity (PFWS) at maximum frown to assess the severity of the glabellar lines at the Screening Visit, Treatment Visit (Day 0) pre-treatment, Follow-up Visits (Weeks 2, 4, 8, 12, 16, 20, 24, 28, 32), and End-of-Study Visit (Week 24, 28, 32, or 36, as appropriate) or Early Discontinuation Visit, if applicable. The assessment form was provided directly to the subject to complete while reviewing the glabellar lines using a supplied handheld mirror. The PFWS rating score system was as follows: a PFWS rating score of (0) indicated no wrinkle severity, with associated description of “no wrinkles;” a PFWS rating score of (1) indicated mild wrinkle severity, with associated description of “very shallow wrinkles;” a PFWS rating score of (2) indicated moderate wrinkle severity, with associated description of “moderate wrinkles;” and a PFWS rating score of (3) indicated “severe wrinkle” severity, with associated description of “deep wrinkles.” In accordance with the study, an IGA-FWS and a PFWS rating of (2) moderate or (3) severe for a subject's glabellar lines were needed for a subject to be enrolled.

Subjects were randomized in a 2:1 ratio to either RT002 or placebo treatment group, respectively. Subjects enrolled in the study had screening and treatments visits and follow-up safety and efficacy evaluations throughout the study for at least 24 weeks and up to 36 weeks, post-treatment. A single administration is given at Week 0. Patients return for follow up visits on Weeks 1, 2, 4 (the primary endpoint), and every 4 weeks thereafter, with Week 24 begin the last mandatory visit, and continuing every 4 weeks until Week 36, the Final Visit.

Patients captured their assessment of the appearance of the lines at maximum frown, in a diary for the initial 2-week post treatment period, using the 4 point severity scale, described herein. The onset of treatment effect was determined based on the patient's diary evaluating the severity of glabellar lines over time within the first two weeks post treatment. The onset of treatment effect was defined as the time when patient rating score drops 1 point or greater from baseline. This was included in the secondary endpoint assessment.

### (a) Efficacy and Safety Assessments

The primary efficacy assessments included investigator assessment of glabellar line severity and glabellar line improvement, and patient assessment of glabellar line severity and improvement.

### (a) Frown Wrinkle Severity—Patient and Investigator Global Assessment

Frown wrinkle severity was assessed by both the patient (Patient Frown Wrinkle Severity [PFWS]) and the investigator (Investigator Global Assessment Frown Wrinkle Severity [IGA-FWS]) using the 4-point rating scale shown in Table 7. The severity is assessed at maximum frown and at rest after maximum frown by both the patient and the investigator. The scores range from 0=none to 3=severe.

TABLE 7
Frown Wrinkle Severity

| Rating | Frown | |
|---|---|---|
| Score | Wrinkle Severity | Description |
| 0 | None | No wrinkles |
| 1 | Mild | Very shallow wrinkles |
| 2 | Moderate | Moderate wrinkles |
| 3 | Severe | Deep wrinkles |

### Primary Efficacy Endpoint

The primary efficacy endpoint was derived from the maximum frown scores obtained at Week 4, and was defined as achieving a score of 0 or 1 (none or mild) and an improvement of at least two points from baseline on both the IGA-FWS and PFWS scales concurrently. The response was abbreviated as “2-point composite response.”

### (a) Secondary Efficacy Endpoints

The secondary endpoints, derived from the IGA-FWS and PFWS assessments at maximum frown, are described below. For endpoints that were assessed in patients who met the 2-point composite response at Week 4, the observed cases data at maximum frown should be used to make this assessment.

(1) Proportion of patients who achieve a score of 0 or 1 (none or mild) on IGA-FWS at Weeks 1, 2, 4, 8, 12, 16, 20, 24, 28, 32, and 36.

(2) Proportion of patients who achieve a score of 0 or I (none or mild) on both IGA-FWS and PFWS at Weeks 1, 2, 4, 8, 12, 16, 20, 24, 28, 32, and 36.

(3) Proportion of patients who achieve a 2-point composite response at Weeks 1, 2, 4*, 8, 12, 16, 20, 24, 28, 32, and 36. (*This is a primary endpoint).

(4) Proportion of patients who achieve a score of 0 or I (none or mild) on PFWS at Weeks 1, 2, 4, 8, 12, 16, 20, 24, 28, 32, and 36.

(5) Time to loss of 2 points or greater response on both IGA-FWS and PFWS, for patients who met the 2-point composite response at Week 4 and are in the RT002 group.

(6) Time to loss of 2 points or greater response on either IGA-FWS and PFWS, for patients who met the 2-point composite response at Week 4 and are in the RT002 group.

(7) Time to return to, or worse than baseline on both IGA-FWS and PFWS, for patients who met the 2-point composite response at Week 4 and are in the RT002 group.

(8) Time to return to, or worse than baseline on both IGA-FWS and PFWS, for all patients in the RT002 group.

(9) Time to return to moderate or severe on both IGA-FWS and PFWS, for patients who met the 2-point composite response at Week 4 and are in the RT002 group.

(10) Time to return to moderate or severe on both IGA-FWS and PFWS, for all patients in the RT002 group

(11) Time to return to moderate or severe on IGA-FWS, for patients who met the 2-point composite response at Week 4 and are in the RT002 group.

(12) Time to return to moderate or severe on IGA-FWS, for all patients in the RT002 group at maximum frown

### (a) Exploratory Efficacy Endpoints

The exploratory endpoints, derived from the IGA-FWS and PFWS assessments at maximum frown, are:

(1) Proportion of patients who achieve an improvement of at least one point on both IGA-FWS and PFWS concurrently (abbreviated henceforth as“I-point composite response”) at each visit from Week 1 to Week 36.

(2) Proportion of patients who achieve an improvement of at least one point on IGA-FWS at each visit from Week 1 to 36.

(3) Proportion of patients who achieve an improvement of at least one point on PFWS at each visit from Week 1 to 36.

(4) In the subset of patients who achieve a 2-point composite response at Week 4, the proportion of patients who achieve a 2-point composite response at each visit from Week 1 to 36.

(5) In the subset of patients who achieve a 2-point composite response at Week 4, the proportion of patients who achieve a 1-point composite response at each visit from Week 1 to Week 36.

(6) In the subset of patients who achieve a 2-point composite response at Week 4, the proportion of patients who achieve a score of 0 or 1 (none or mild) on both IGA-FWS and PFWS at each visit from Week 1 to 36.

(7) Proportion of patients who achieve an improvement of at least 2-points on IGA-FWS at each visit from Week 1 to 36.

(8) Proportion of patients who achieve an improvement of at least 2-points on PFWS at each visit from Week 1 to 36.

### (a) Additional Assessments

Patient Diary: Patients captured their assessment of the appearance of the lines at maximum frown, in a diary for the initial 2-week post treatment period, using the 4 point severity scale described above for Frown Wrinkle Severity. The onset of treatment effect was determined based on the patient's diary evaluating the severity of glabellar lines over time within the first two weeks post treatment. The onset of treatment effect was defined as the time when patient rating score drops 1 point or greater from baseline. This was included in the secondary endpoint assessment.

Patient Global Satisfaction with Treatment Questionnaire: Patients were asked how satisfied or dissatisfied they are with the treatment results using a 7-point scale at Week 4. This treatment questionnaire was based on how the treated area of the face looks, according to Table 8. The rating score was used as a secondary endpoint.

TABLE 8
Global Satisfaction with Treatment
Questionnaire Scale

| Rating | |
|---|---|
| Score | Wrinkle Improvement |
| 0 | Very Dissatisfied |
| 1 | Dissatisfied |
| 2 | Somewhat Dissatisfied |
| 3 | Neither Satisfied Nor Dissatisfied |
| 4 | Somewhat Satisfied |
| 5 | Satisfied |
| 6 | Very Satisfied |

Global Aesthetic Improvement Scale: The Investigator and patient assessed the visual appearance (at maximum frown and at rest after maximum frown) of the glabellar line improvement from the baseline condition using the following 7 point severity Global Aesthetic Improvement Scale, as shown in Table 9.

TABLE 9
Global Aesthetic Improvement Scale

| Rating | Wrinkle |
|---|---|
| Score | Improvement |
| −3 | Very Much Worse |
| −2 | Much Worse |
| −1 | Worse |
| 0 | No Change |
| 1 | Improved |
| 2 | Much Improved |
| 3 | Very Much Improved |

The exploratory efficacy endpoints derived from this assessment are:

Proportion of patients who achieve a score of 2:1 on GAIS at each visit from Week 1 to Week 36 (with investigator's assessment at maximum frown, at rest after maximum frown and patient's self-assessment at maximum frown, at rest after maximum frown, summarized separately)

(2) Proportion of patients who achieve a score of 2:2 (i.e., much improved, or very much improved) on GAIS at each visit from Week 1 to Week 36 (with investigator's assessment at maximum frown, at rest after maximum frown and patient's self-assessment at maximum frown, at rest after maximum frown, summarized separately)

(3) Proportion of patients who achieve a score of 2:3 (very much improved) on GAIS at each visit from Week 1 to Week 36 (with investigator's assessment at maximum frown, at rest after maximum frown and patient's self-assessment at maximum frown, at rest after maximum frown, summarized separately)

(4) GAIS score over time from Week 1 to Week 36 (with investigator's assessment at maximum frown, at rest after maximum frown and patient's self-assessment at maximum frown, at rest after maximum frown, summarized separately)

Frown Line Impact Scale: Patients were asked to rate their feelings about the treatment results on their frown lines using the Frown Line Impact Scale (FLIS). The FLIS is comprised of 5 questions, each with an 11 point scale ranging from 0 to 10. The total score, ranging from 0 to 50, is the sum of the scores of the 5 questions. The exploratory endpoints were the total score and the scores on the individual questions.

Facial Age Self Evaluation: Patients rated their perceived age on a Facial Age Self Evaluation (FASE) questionnaire and rated their perception of how old they think they look following the treatment (older than actual age, younger than actual age, actual age). These responses were used as exploratory endpoints.

Photographs: For those patients consenting to photography, standardized digital photographs of the treatment area were taken, including the patient's frontal view at maximum frown and at rest after maximum frown. The photographs were scored for severity of the glabellar lines utilizing the IGA-FWS of the Frown Wrinkle Severity Table (above) and via Independent Panel Review (IPR). In addition, a 2-point response, defined as achieving a score of 0 or 1 (none or mild) and an improvement of at least two points from baseline on the IGA-FWS at Week 4 was determined.

### (a) Safety Assessments

Adverse Events: All adverse events (AEs) were recorded and classified on the basis of MedDRA terminology. AE severity was graded as mild, moderate, or severe. AEs with an onset on or after the date and time of study treatment were Treatment-emergent.

The safety endpoints derived from the AEs were:

(1) Frequency, severity and relationship to study drug of treatment-emergent adverse events during the first four weeks post treatment and the overall study duration

(2) Frequency, severity and relationship to study drug of treatment-emergent serious adverse events during the first four weeks post treatment and the overall study duration.

Distant Spread of Toxin Query (Specific AE and Symptoms of Neuromuscular Weakness Query): Distant Spread of Toxin Query was conducted at the Treatment Visit pre- and post-treatment, Follow-up Visits, and Final Evaluation Visit or Early Discontinuation Visit, if applicable. Patients were queried in a general manner on the list of adverse events potentially suggestive of distant spread of toxin.

Clinical Laboratory Data: Non-fasting samples for hematology, chemistry, coagulation (prothrombin time) and urinalysis were collected at Screening, Week 4, and at the Final Evaluation Visit. At Screening and Week 2, 4, and 12 Visits blood samples for antibodies were collected.

TABLE 10
Clinical Laboratory Tests

| Serum | | | |
|---|---|---|---|
| Chemistry | Hematology | Urinalysis | Additional Tests |
| Glucose | Hemoglobin | Overall | Prothrombin time (PT) |
| Total bilirubin | Hematocrit | Assessment | Urine Pregnancy |
| Alkaline | Red Blood Cell | and Clinical | (WOCBP* only) Serum |
| phosphatase | Count | Significance | antibodies for |
| Blood urea | Platelet Count | | daxibotulinumtoxinA |
| nitrogen | Leukocyte | | and |
| Alanine | Count (total) | | |
| aminotransferase | Leukocyte | | |
| Aspartate | Count | | |
| aminotransferase | (differential) | | |

*WOCBP = Women of child-bearing potential

Antibody Testing: Antibody testing for RT002 and RTP004 was performed qualitatively using a screening assay, and if positive, was tested by a confirmation assay. The confirmation assay resulted in both a qualitative assessment (positive/negative) as well as a quantitative concentration if positive. Samples testing positive by the confirmation assay, will also be testing for neutralizing antibody.

Vital Signs: Vital signs (i.e., body temperature, respiration rate, sitting radial pulse rate, and sitting systolic and diastolic blood pressures) were obtained at the Screening and Treatment Visit (pre- and post-treatment), Week 2, Final Evaluation or Early Discontinuation Visits and at any visit where signs or symptoms of botulinum toxicity were reported.

Physical Examination: A physical examination, in addition to vital signs, including neurological examination of the face, general appearance, skin, neck (including thyroid), eyes, ears, nose, throat, heart, lungs, abdomen, lymph nodes, and extremities was conducted at Screening, Week 2 and Final Evaluation or Early Discontinuation Visits. Significant physical examination findings that are present prior to investigational product administration are to be included on the Medical History page. Significant physical examination findings which meet the definition of an adverse event were recorded.

12-Lead ECG: At Screening and Week 4, a single standard supine 12-Lead ECG was obtained.

Injection Site Evaluation: Injection sites were evaluated at the Screening Visit, Treatment Visit pre- and post-treatment, Follow-up Visits, and Final Evaluation Visit or Early Discontinuation Visit, if applicable. The assessment will be done as a global evaluation of the 5 injection sites, as shown in Table 11.

TABLE 11
Injection Site Evaluation

| | Present? | |
|---|---|---|
| Assessment Descriptor | Yes | No |
| Erythema | | |
| Edema | | |
| Burning or Stinging | | |
| (sensation as described by patient) | | |
| Itching | | |
| (sensation as described by patient) | | |
| Bruising | | |

Assessment of Cranial Nerves II-VII: Evaluation of cranial nerves II-VII (left and right sides separately) was performed by the Investigator at Screening, Treatment Visit pre- and post-treatment, Follow-up Visits and Final Evaluation Visit or Early Discontinuation Visit, if applicable. Scores for each cranial nerve were captured as outlined in Table 12.

TABLE 12
Cranial Nerve Assessment

| Rating | Description |
|---|---|
| 1 | Normal |
| 2 | Abnormal, not clinically significant |
| | Abnormal, clinically significant |
| 3 | Not assessed |
| 4 | Description |

Regional House-Brackmann Facial Nerve Grading System: The Investigator evaluated functionality of the facial nerve (VII) at Screening, Treatment Visit pre- and post-treatment, Follow-up Visits, and Final Evaluation Visit or Early Discontinuation Visit, if applicable. Refer to Table 13:

TABLE 13
Regional House-Brackmann Facial Nerve Grading System

| | |
|---|---|
| Forehead | 1 Normal forehead movement |
| | 2 Slight weakness in forehead movement |
| | 3 Obvious but not disfiguring asymmetry |
| | with motion, symmetric at rest |
| | 4 Obvious weakness of disfiguring asymmetry |
| | with motion, symmetric at |
| | 5 Barely perceptible motion in forehead, |
| | asymmetric at rest |
| | 6 No movement |
| Eye | 1 Normal eye closure |
| | 2 Mild weakness in eye closure |
| | 3 Obvious weakness but able to close eyes |
| | 4 Unable to close eye with maximal effort |
| | 5 Barely perceptible eyelid movement |
| | 6 No movement |
| Midface | 1 Normal midface movement |
| | 2 Slight weakness in midface movement |
| | 3 Obvious but not disfiguring weakness, |
| | symmetric at rest |
| | 4 Obvious weakness and disfiguring asymmetry |
| | with motion, symmetric at |
| | 5 Barely perceptible motion in midface, |
| | asymmetric at rest |
| | 6 No movement |
| Mouth | 1 Normal corner of mouth movement |
| | 2 Slight weakness of corner of mouth movement |
| | 3 Obvious but not disfiguring weakness, symmetric |
| | at rest |
| | 4 Obvious weakness and disfiguring asymmetry |
| | with motion, symmetric at |
| | 5 Barely perceptible corner of mouth movement, |
| | asymmetric at rest |
| | 6 No movement |
| Synkinesis | 1 None |
| | 2 Mild-obvious but not disfiguring |
| | 3 Severe-disfiguring or interferes with function |

Evaluation of Facial Muscle Strength: Facial muscle strength was evaluated using the Medical Research Council (MRC) Scale for Assessment of Muscle Power. The following muscles on each side of the face were evaluated: orbicularis oculi (eyelid), lateral brow elevators, zygomaticu. See Table 14. The Investigator evaluated facial muscle strength at Treatment Visit pre- and post-treatment, Follow-up Visits and Final Evaluation Visit or Early Discontinuation Visit, if applicable.

TABLE 14
MRC Scale for Assessment of Muscle Power

| Rating | |
|---|---|
| Scale | Description |
| 0 | no movement |
| 1 | flicker is perceptible in the muscle |
| 2 | movement only if gravity eliminated |
| 3 | can move limb against gravity |
| 4 | can move against gravity and some |
| | resistance exerted by examiner |
| 5 | normal power |

Statistical Analysis: All statistical programming was performed using statistical analysis system (SAS) version 9.3 or higher.

Most of the efficacy endpoints were analyzed with trial center as a stratification factor. The trial was intended to be conducted in a manner such that a minimum of 5 ITT patients in each treatment group were enrolled at each trial center/site. In the event that there were too few patients in a treatment arm at a single site, this site was combined with another to achieve the desired minimum sample size per arm. Small centers (<5 ITT patients in a treatment group) were pooled from largest to smallest until the pooled center has 2:5 ITT patients in each treatment group (William et al, 2006, “Effects of a New Hormone Therapy, Drospirenone and 17—Estradiol, in Postmenopausal Women With Hypertension”, Hypertension, 48:246-253). If any centers needed to be pooled, then any analysis performed by trial center was performed by pooled center instead.

### (a) Analysis Populations

Intent-to-Treat Population: All patients who were randomized and received treatment are included in the Intent-to-Treat (ITT) population. The summaries were by treatment as randomized.

Per Protocol Population: The Per-Protocol (PP) population included patients from the ITT population who complete the first 4-weeks of the study without a major protocol violation. Decisions about exclusions from the PP population were made prior to unblinding the study, with the exception of patients who received the incorrect dose/treatment. Patients were excluded from the PP population for any of the following reasons: patient violates inclusion/exclusion criteria; patient receives incorrect dose; patient receives incorrect treatment; patient uses a prohibited medication prior to Week 4; patient misses the Week 4 visit; patient's Week 4 visit is greater than ±3 days out of window; patient is missing either the IGA-FWS or PFWS evaluation at Week 4.

Safety Population: All patients who were randomized, received treatment, and provided at least one post-treatment safety assessment were included in the Safety population. The summaries were by treatment actually received.

A summary of the duration of the patient participation in the study was produced, including then, mean, SD, median, minimum, and maximum duration in weeks, as well as the number and percentage of patients in the following categories of duration: <4 weeks, 4 to <12 weeks, 12 to <24 weeks, and 24 to 36 weeks.

Demographic and Baseline Characteristics: Descriptive statistics were used to summarize demographic and baseline characteristics by treatment group and overall. Continuous variables were summarized using the number of non-missing observations, mean, standard deviation, median, minimum and maximum. Categorical data were summarized using the number and percentage of patients in each category.

Demographic data include age, sex, race and ethnicity. Age in years were categorized as 18 to 45, >45 to 55, and >55 to 75 for summarizing by treatment group and overall. Baseline characteristics include Prior Botulinum Toxin Type A, Time Since Last Prior Botulinum Toxin Type A Injection, and Fitzpatrick Skin Type, as well as the baseline assessment of the efficacy questionnaires, PFWS, IGA-FWS, FLIS, and FASE. Summaries were produced for the ITT and PP populations by randomized treatment; and, for the Safety population by actual treatment received.

Efficacy Analyses: Descriptive statistics were provided for all efficacy variables at all-time points by treatment group.

Unless specified otherwise, the main method of handling missing efficacy data was based on the patient-level worst outcome imputation for the RT002 group (RT002 group) and the patient-level best outcome imputation for the placebo group (worst/best-outcome imputation). According to the study design, all patients were to be followed for a minimum of 24 weeks. For this reason, the imputation was done only through the week 24 visit. For endpoints that are composite and/or derived from study assessments, imputation of missing data was performed on the original assessments first.

A model-based multiple imputation approach also was used as an additional sensitivity analysis. Patients who violated inclusion/exclusion criteria due to using prohibited medication after week 4 might have been included in the above sensitivity analysis if they added up to 10% or more of the study patients.

Multiplicity adjustment among the secondary endpoints was assessed using a Type I Error Control Plan. Non-adjusted p-values were provided to guide the hypothesis testing rules in the Type I Error Control Plan.

Primary Efficacy Analysis: The proportion of patients who had a 2-point composite response at Week 4 was compared between RT002 and placebo using the Cochran-Mantel-Haenszel (CMH) test stratified by trial center using a two-sided test with a Type I error rate of 0.05 using the ITT population with worst/best outcome imputation. As a sensitivity analysis, the primary analysis was repeated using multiple imputation instead of worst/best outcome imputation in the ITT population. As an additional sensitivity analysis, the CMH test was performed using the observed cases only in the PP population. The Breslow-Day test was computed to test for the homogeneity of the odds ratios.

P-values based on 2-sided CMH tests were provided. The point estimates for the difference were calculated with the Mantel-Haenszel estimate of the common risk difference. To check for consistency, the summary score estimate of the common risk difference also was calculated and compared to the Mantel-Haenszel estimate, but was not reported in the tables. The 2-sided, 95% Cls were calculated with the stratified Newcombe confidence limits for the common risk difference, using the method of Yan and Su (2010). The stratified Newcombe confidence limits were constructed from stratified Wilson confidence limits for the common (overall) row proportions. First the individual Wilson confidence limits were computed for the row proportions in each 2×2 table (stratum). These stratum Wilson confidence limits were then combined to form stratified Wilson confidence limits for the overall row proportions by using Mantel-Haenszel weights.

Secondary Efficacy Analyses: There are multiple secondary endpoints. They were grouped based on the statistical analysis method applied to them. There were 4 different groups of secondary endpoints, Groups A, B, C, and D, with different statistical tests for each group. The imputation of the secondary endpoints were done using the ITT population with worst/best-outcome imputation for Groups A and B. Multiple imputation also was performed on the endpoints in Groups A and Bin the ITT population. For Groups A, B and C, the analyses conducted were for all patients, as well as patients by baseline severity (moderate or severe, per IGA-FWS assessment), to assess the magnitude of the treatment effect of patient wrinkle severity at baseline on these outcome measures.

Group A: Reduction in severity over time was evaluated for the following secondary efficacy endpoints that were in the form of proportion of patients having a response using various response definitions. For each endpoint at selected visits, the proportion of patients with a response was compared between treatment groups using the CMH test stratified by the trial center. The p-values, point estimates and 95% Cis were computed using the same methods as with the primary endpoint.

(1) The proportion of patients who achieved a score of 0 or 1 (none or mild) on IGA-FWS at maximum frown by selected visit (Weeks 2, 4, 8, 12, 16, 20, and 24) using (a) Worst/best outcome imputation in the ITT population; (b) Multiple imputation in the ITT population; (c) Observed cases in the PP population.

(2) The proportion of patients who achieved a score of 0 or 1 (none or mild) on both IGA-FWS and PFWS at maximum frown by selected visit (Weeks 2, 4, 8, 12, 16, 20, and 24) using (a) Worst/best outcome imputation in the ITT population; (b) Multiple imputation in the ITT population; (c) Observed cases in the PP population.

Group B: For the secondary endpoints in this group, the proportion of responders and the difference in proportions, with a 90% Wald CI were summarized by treatment group and visit. The two group A endpoints were included in Group B so as to include all weeks and to provide the 90% Wald CI for these endpoints.

(1) The proportion of patients who achieve a 2-point composite response at each visit (Weeks 1, 2, 4, 8, 12, 16, 20, 24 28, 32, and 36) using (a) Worst/best outcome imputation in the ITT population; (b) Multiple imputation in the ITT population; (c) Observed cases in the PP population; (d) Worst/best outcome imputation in the ITT population by Baseline IGA-FWS severity.

(2) The proportion of patients who achieved a score of 0 or 1 (none or mild) on both IGA-FWS and PFWS at each visit (Weeks 1, 2, 4, 8, 12, 16, 20, 24, 28, 32, and 36) using (a) Worst/best outcome imputation in the ITT population; (b) Multiple imputation in the ITT population; (c) Observed cases in the PP population; (d) Worst/best outcome imputation in the ITT population by Baseline IGA-FWS severity.

(3) The proportion of patients who achieved a score of 0 or 1 (none or mild) on IGA-FWS at each visit (Weeks 1, 2, 4, 8, 12, 16, 20, 24, 28, 32, and 36) using (a) Worst/best outcome imputation in the ITT population; (b) Multiple imputation in the ITT population; (c) Observed cases in the PP population; (d) Worst/best outcome imputation in the ITT population by Baseline IGA-FWS severity.

(4) The proportion of patients who achieve a score of 0 or 1 (none or mild) on PFWS at each visit (Weeks 1, 2, 4, 8, 12, 16, 20, 24, 28, 32, and 36) using (a) Worst/best outcome imputation in the ITT population; (b) Multiple imputation in the ITT population; (c) Observed cases in the PP population; (d) Worst/best outcome imputation in the ITT population by Baseline IGA-FWS severity.

Group C: Kaplan-Meier survival curves were plotted for the RT002 group for the following time-to-event endpoints. Point estimates of median duration and 2-sided, 95% Cls, using the log-log transformation, were generated. Estimates of survival rates and the 2-sided, 95% CI, using the log-log transformation, of the rate at Weeks 8, 12, 16, 20 and 24 also were provided. Rates for Weeks 2 and 4 were included in analyses involving all patients in the RT002 group. All analyses were performed for the ITT population using observed cases.

(1) Time to loss of 2 points or greater response on both IGA-FWS and PFWS,

(a) for patients who met the 2-point composite response at Week 4 and were in the RT002 group at maximum frown; (b) for patients who met the 2-point composite response at Week 4 and were in the RT002 group at maximum frown by Baseline IGA-FWS severity.

(2) Time to loss of 2 points or greater response on either IGA-FWS or PFWS, (a) for patients who met the 2-point composite response at Week 4 and were in the RT002 group at maximum frown; (b) for patients who met the 2-point composite response at Week 4 and were in the RT002 group at maximum frown by Baseline IGA-FWS severity.

(3) Time to return to, or worse than baseline on both IGA-FWS and PFWS, (a) for patients who met the 2-point composite response at Week 4 and were in the RT002 group at maximum frown; (b) for patients who met the 2-point composite response at Week 4 and were in the RT002 group at maximum frown by Baseline IGA-FWS severity; (c) for all patients in the RT002 group at maximum frown; (d) for all patients in the RT002 group at maximum frown by Baseline IGA-FWS severity.

(4) Time to return to moderate or severe on both IGA-FWS and PFWS, (a) for patients who met the 2-point composite response at Week 4 and were in the RT002 group at maximum frown; (b) for patients who met the 2-point composite response at Week 4 and were in the RT002 group at maximum frown by Baseline IGA-FWS severity; (c) for all patients in the RT002 group at maximum frown; (d) for all patients in the RT002 group at maximum frown by Baseline IGA-FWS severity.

(5) Time to return to moderate or severe on IGA-FWS, (a) for patients who met the 2-point composite response at Week 4 and were in the RT002 group at maximum frown; (b) for patients who met the 2-point composite response at Week 4 and were in the RT002 group at maximum frown by Baseline IGA-FWS severity; (c) for all patients in the RT002 group at maximum frown; (d) for all patients in the RT002 group at maximum frown by Baseline IGA-FWS severity.

Group D: The endpoints in Group D were summarized descriptively as follows:

(1) The patient global satisfaction with treatment questionnaire were summarized using the number and percentage of patients with each response. The 2-sided, 90% CI, using asymptotic Wald confidence intervals, will were presented.

(2) The onset of treatment effect, from the patient diary, was summarized using the number and percentage of patients with an onset of effect and without an onset of effect. In addition, descriptive statistics (median, minimum, maximum, 25th and 75th quartiles) of the onset time were presented for the patients with an onset of treatment effect. Onset of treatment effect was restricted to the first 14 days of the study.

Exploratory Efficacy Analyses: All exploratory efficacy endpoints are summarized descriptively by treatment group and by visit. Treatment group comparisons are performed for some exploratory endpoints using the methods similar to those specified for the primary and secondary efficacy endpoints. The analyses of the exploratory endpoints are done on the ITT population for the observed data only.

For each exploratory endpoint derived from the IGA-FWS and PFWS assessments at each visit, the proportion of patients with a response is compared between treatment groups using the CMH test stratified by the trial center. The p-values, point estimates and 95% Cis are computed using the same methods as with the primary endpoint.

Kaplan-Meier survival curves of the time to loss of at least a 1 point improvement from baseline are plotted as above (see Group C). Point estimates of median duration and 2-sided, 95% Cis, using the log-log transformation, are generated. This analysis will be performed for the following:

(1) Time to loss of at least a 1 point improvement from baseline in IGA-FWS, (a) for all patients in the RT002 group at maximum frown; (b) for patients who achieved at least a 1 point improvement from baseline in IGA-FWS and are in the RT002 group at max frown.

(2) Time to loss of at least a 1 point improvement from baseline in PFWS, (a) for all patients in the RT002 group at maximum frown; (b) for patients who achieved at least a 1 point improvement from baseline in PFWS and are in the RT002 group at maximum frown.

(3) Time to loss of at least a 1 point improvement from baseline in both IGA-FWS and PFWS, (a) for all patients in the RT002 group at maximum frown; (b) for patients who achieved at least a 1 point improvement from baseline in both IGA-FWS and PFWS and are in the RT002 group at maximum frown.

For the endpoints derived from the GAIS assessment, the number and percentage of patients who achieve scores of 2:1, 2:2, and 2:3 are summarized by treatment group and visit. For the GAIS score, the number and percentage of patients at each scale level are summarized by treatment group and visit. The investigator and subject assessments are summarized separately for both the assessment performed at maximum frown and the assessment performed at rest after maximum frown.

The FLIS total score and the scores of the individual questions are summarized descriptively by treatment group and visit with n, mean (SD), SEM, median, minimum, and maximum.

For the FASE questionnaire, the “What Age Do You Think You Look Right Now?”, Number of Years Older, and Number of Years Younger questions, is summarized with n, mean (SD), SEM, median, minimum, and maximum. For the “Do You Feel Like You Look Older Or Younger Than Your Actual Age Right Now?” question, the number and percentage of patients in each category are summarized by treatment group and visit.

The IGA-FWS evaluations of the photographs assessed by Independent Panel review (IPR) are summarized by the number and percentage of patients at each severity level by treatment group and visit, at maximum frown. In addition, the number and percentage of patients with a 2-point response on the IGA-FWS at Week 4 are provided for each treatment group and the difference between the treatment groups is presented, along with the 2-sided, 90% Wald CI for the difference.

Examination of Subgroups: Subgroup analyses for the primary endpoint and key secondary endpoints associated with IGA-FWS and/or PFWS assessments are performed with subgroups classified by prior treatment with Botulinum Toxin Type A (yes/no), gender, and age group (two age group subgroups will be used: 18-45 Years, >45-55 Years, >55-75 Years, and 18-65 Years, >=65 Years). Specifically, the following endpoints are examined by subgroup:

(1) Proportion of patients who achieve a 2-point composite response; (2) Proportion of patients who achieve a score of 0 or 1 (none or mild) on both IGA-FWS and PFWS; (3) Proportion of patients who achieve a score of O or 1 (none or mild) on IGA-FWS; (4) Proportion of patients who achieve a score of O or 1 (none or mild) on PFWS; (5) Response on the patient global satisfaction with treatment questionnaire; (6) The onset of treatment effect, derived from the patient diary.

Because subgroup analyses have inherently lower statistical power than analyses of the full cohort, only descriptive statistics for the individual treatment groups are presented. Subgroup analyses on the primary endpoint and the secondary endpoints in Groups A and B are performed on the ITT population with worst/best-outcome imputation. Subgroup analyses on the Group C and D secondary endpoints using observed data on the ITT population also are summarized.

### (a) Safety Analyses

Safety summaries and analyses were performed on the safety population. Descriptive statistics were presented to summarize the safety data.

Extent of Exposure: All patients received one administration of investigational product. The sum of volume of investigational product injected and the volume of investigational product injected at each of the five injection sites were summarized by treatment group using descriptive statistics (number of non-missing observations, mean, median, minimum, maximum, and standard deviation).

Injection Site Evaluations: The injection site evaluations were summarized using number and percentage of patients reporting the presence of each item (Erythema, Edema, Burning or Stinging, Itching and Bruising) by treatment group and visit, as well as the number and percentage of patients with a reaction at any post-treatment visit. In addition, the number and percentage of patients reporting any injection site item was summarized by treatment group, and by visit as well as at any post-treatment visit. Additionally, the number and percentages of patients with the specified item were summarized according to the first visit at which the reaction was present.

Adverse Events: All treatment-emergent AEs (TEAEs) were listed and summarized by treatment group, system organ class, preferred term, severity, relationship, and seriousness. Serious adverse events (SAEs) were summarized by treatment group, severity, and relationship to study treatment and listed by patient separately.

Nerve Evaluations: Cranial Nerves II-VII examinations, for functions Pupillary Reaction to Light and Accommodation, Extraocular Movements, Motor, and Gross Sensation, for Right and Left sides each, were tabulated by treatment group and visit.

The Regional House-Brackmann Facial Nerve Grading System (Yen, 2003) was tabulated for the right and left sides of the four major branches of the facial nerve (VII) that innervates target and adjacent musculature (forehead, eye, midface, and mouth) and severity of Synkinesis. These tabulations were by treatment group and visit.

Facial muscle strength of the right and left sides each of Orbicularis Oculi, Lateral Brow Elevators, and Lateral Orbicularis Zygomaticus, was tabulated by treatment group and visit.

Changes in the Conduct of the Study or Planned Analyses: Changes to the planned analyses include the method of handling missing data for the placebo group, selection and prioritization of the secondary endpoints, and the testing procedure to control the overall study Type-I error. Specifically, the following changes to the endpoints were made:

(1) IGA-FWS and PFWS responder analyses that were split between secondary and exploratory based on week of assessment are all secondary now.

(2) Onset of treatment effect based on the patient diary and the patient global satisfaction with treatment have been moved from exploratory to secondary.

(3) GAIS endpoints are all exploratory, rather than some being considered secondary.

(4) Additional exploratory endpoints based on the IGA-FWS and/or PFWS have been included: (a) proportion of patients who achieve a score of O or 1 (none or mild) on both IGA-FWS and on PFWS concurrently; (b) proportion of patients who achieve an improvement of at least one point on IGA-FWS and on PFWS, separately; (c) proportion of patients who achieve an improvement of at least two points on IGA-FWS and on PFWS, separately; (d) proportion of patients who achieve a 1- or 2-point composite response in the subset of patients who achieve a 2-point composite response at Week 4; (e) proportion of patients who achieve scores of O or 1 (none or mild) on both IGA-FWS and on PFWS, in the subset of patients who achieve a 2-point composite response at Week 4.

(5) Two additional time to event endpoints, based on the IGA-FWS and/or PFWS, have been included: (a) Time to loss of 2 points or greater response on both IGA-FWS and PFWS (b) Time to loss of 2 points or greater response on either IGA-FWS or PFWS (c) Time to return to moderate or severe on IGA-FWS.

(6) The exploratory endpoint of “Glabellar line improvement per GAIS assessment at rest after maximum frown over time” is removed as this endpoint is assessed by the proportion of patients with a score >=1, >=2 and >=3 over time, in addition to the mean score overtime.

Assessments on Time to Return also were made. Time to return to, or worse than, baseline on both IGA-FWS and PFWS is assessed using a number of measures, including (1) the number of days from treatment start date to the first visit at which both IGA-FWS and PFWS return to, or worse than, baseline following the later of Weeks 1, 2, and 4 at which an improvement from baseline in both is observed. If no such visit is present, censoring occurs at the latest visit with both IGA-FWS and PFWS available. If there is no improvement from baseline in both IGA-FWS and PFWS at Weeks 1, 2, and 4, then time is set to 0; and (2) he number of days from the first improvement from baseline on both IGA-FWS and PFWS to the earliest subsequent return to, or worse than, baseline on both on both IGA-FWS and PFWS. If no subsequent return to, or worse than, baseline is observed censoring occurs at the latest visit with both IGA-FSW and PFWS available. For patients who do not improve from baseline on both IFA-FWS and PFWS at any visit on or after week 4, time is set to 0.

### (i) Study Results: Phase 3 Topline Results of two Study Arms for Glabellar Lines at Week 36

Subject Disposition: Table 15 describes the disposition of the ITT, Safety and Per-Protocol populations in both arms of the study.

TABLE 15

| | Arm 1 | | Arm 2 | |
|---|---|---|---|---|
| | | RT002 | | RT002 |
| | Placebo | 40 U | Placebo | 40 U |
| | (n = 102) | (n = 201) | (n = 102) | (n = 204) |
| Completed | 97 (95.1%) | 196 (97.5%) | 99 (97.1%) | 203 (99.5%) |
| Week 4 | | | | |
| Completed | 91 (89.2%) | 188 (93.5%) | 93 (91.2%) | 199 (97.5%) |
| Week 24 | | | | |
| Completed | 93 (91.2%) | 182 (90.5%) | 93 (91.2%) | 191 (93.6%) |
| the Study | | | | |
| Early | 9 (8.8%) | 19 (9.5%) | 9 (8.8%) | 13 (6.4%) |
| Discontinuation | | | | |
| Withdrew consent | 4 (3.9%) | 8 (4.0%) | 5 (4.9%) | 9 (4.4%) |
| Lost to follow-up | 4 (3.9%) | 6 (3.0%) | 3 (3.9%) | 1 (0.5%) |
| Protocol deviation | 1 (1.0%) | 0 | 0 | 1 (0.5%) |
| Investigator | 0 | 1 (0.5)% | 1 (1.0%) | 1 (0.5%) |
| discretion | | | | |
| Other | 0 | 4 (2.0)% | 0 | 1 (0.5%) |

As Table 15 shows, a high proportion (89.2-97.5%) of the subjects completed the Week 24 visit, and a similarly high proportion (90.5-93.6%) of the subjects completed the studies. Nearly all (95.1-99.5%) of the 609 subjects in both arms of the study completed the Week 4 (primary endpoint) visit. The low early discontinuation frequency (6.4-9.5%) reinforces that the two studies were well executed. Of note, the proportion of subjects excluded from the Per protocol Population is relatively low and ranged between 8.8-22.5%

Analysis of the populations: Table 16 shows analysis of the different populations.

TABLE 16

| | Arm 1 | | Arm 2 | |
|---|---|---|---|---|
| | | RT002 | | RT002 |
| | Placebo | 40 U | Placebo | 40 U |
| | (n = 102) | (n = 201) | (n = 102) | (n = 204) |
| Intent-to-rent (ITT) | 102 (100%) | 201 (100%) | 102 (100%) | 204 (100%) |
| Safety | 102 (100%) | 201 (100%) | 101 (99.0%)* | 205 (100.5%) |
| Per-Protocol (PP) | 79 (77.5%) | 176 (87.6%) | 90 (88.2%) | 186 (91.2%) |
| Excluded from PP | 23 (22.5%) | 25 (12.4%) | 12 (11.8%) | 18 (8.8%) |
| Week 4 visit off schedule | 12 (11.8%) | 15 (7.5%) | 6 (5.9%) | 9 (4.4%) |
| Missed Week 4 visit | 5 (4.9%) | 5 (2.5%) | 3 (2.9%) | 1 (0.5%) |
| No IGA-FWS or PFWS at Week 4 | 5 (4.9%) | 5 (2.5%) | 3 (2.9%) | 1 (0.5%) |
| Used Prohibited Medication Prior | 1 (1.0%) | 1 (0.5%) | 0 | 1 (0.5%) |
| to Week 4 | | | | |
| I/E criteria violation | 6 (5.9%) | 6 (3.0%) | 2 (2.0%) | 7 (3.4%) |
| Received incorrect treatment | 0 | 0 | 1 (1.0%) | 0 |
| Treatment kit out of sequence | 0 | 1 (0.5%) | 0 | 0 |

Demographics: Table 17 shows demographics of the study populations. Both placebo and active cohorts were well balanced with respect to the proportion of female subjects (86.3-89.7%) and a mean age of 50 (49.0-50.9).

TABLE 17

| | Arm 1 | | Arm 2 | |
|---|---|---|---|---|
| | | RT002 | | RT002 |
| | Placebo | 40 U | Placebo | 40 U |
| | (n = 102) | (n = 201) | (n = 102) | (n = 204) |
| Female, n (%) | 88 (86.3%) | 174 (86.6%) | 87 (85.3%) | 183 (89.7%) |
| Age (years), | 49.0 | 50.9 | 50.5 | 49.6 |
| mean (SD), | (11.13) | (11.22) | (9.98%) | (9.84) |
| range | 22, 74 | 23, 74 | 27, 75 | 21, 73 |
| 18 to 45 years | 32 (31.4%) | 58 (28.9%) | 30 (29.4%) | 62 (30.4%) |
| 46 to 55 years | 41 (40.2%) | 68 (33.8%) | 42 (41.2%) | 91 (44.6%) |
| 56 to 75 years | 29 (28.4%) | 75 (37.3%) | 30 (29.4%) | 51 (25.0%) |
| Hispanic/Latino, | 25 (24.5%) | 47 (23.4%) | 10 (9.8%) | 19 (9.3%) |
| n (%) Race | | | | |
| White | 81 (79.4%) | 173 (86.1%) | 92 (90.2%) | 180 (88.2%) |
| Black/African | 8 (7.8%) | 10 (5.0%) | 3 (2.9%) | 9 (4.4%) |
| American | | | | |
| Asian | 2 (2.0%) | 7 (3.5%) | 5 (4.9%) | 11 (5.4%) |
| Other | 11 (10.8%) | 11 (5.5%) | 2 (2.0%) | 4 (2.0%) |

Baseline Characteristics: Table 18 shows percent of prior botulinum toxin use and other characteristics. In Arm 1, the proportion of prior botulinum toxin use was lower (44.1-45.8%) relative to Arm 2 (58.8-59.3%). Baseline values for months since last use of botulinum toxin (22.6-32.2), IGA-FWS at Maximum Frown, and PFWS at Maximum Frown were relatively well balanced in Arms 1 and 2.

TABLE 18

| | Arm 1 | | Arm 2 | |
|---|---|---|---|---|
| | | RT002 | | RT002 |
| | Placebo | 40 U | Placebo | 40 U |
| | (n = 102) | (n = 201) | (n = 102) | (n = 204) |
| Prior | 45 (44.1%) | 92 (45.8%) | 60 (58.8%) | 121 (59.3%) |
| botulinumtoxinA, | | | | |
| n (%) | | | | |
| Months since last | 22.6 | 32.2 | 23.0 | 22.7 |
| BoNT-A* | (19.61) | (37.05) | (24.36) | (23.67) |
| mean (DS) | 1, 94 | 7, 205 | 7, 121 | 7, 193 |
| ranee | | | | |
| IGA-FWS at | | | | |
| Maximum Frown | | | | |
| Moderate | 66 (64.7%) | 123 (61.2%) | 67 (65.7%) | 129 (63.2%) |
| Severe | 36 (35.3%) | 78 (38.8%) | 35 (34.3%) | 75 (36.8%) |
| PFWS At | | | | |
| Maximum Frown | | | | |
| Moderate | 64 (62.7%) | 120 (59.7%) | 49 (48.0%) | 106 (52.0%) |
| Severe | 38 (37.3%) | 81 (40.3%) | 53 (52.0%) | 98 (48.0%) |

Efficacy: Both Arms 1 and 2 pivotal Phase 3 studies with RT002 for injection (RT002; Daxi) at 40 U for the treatment of glabellar lines either exceeded or met results observed in Example 5, a Phase 2 study with 24 Week duration, observed in key response rate and duration measures.

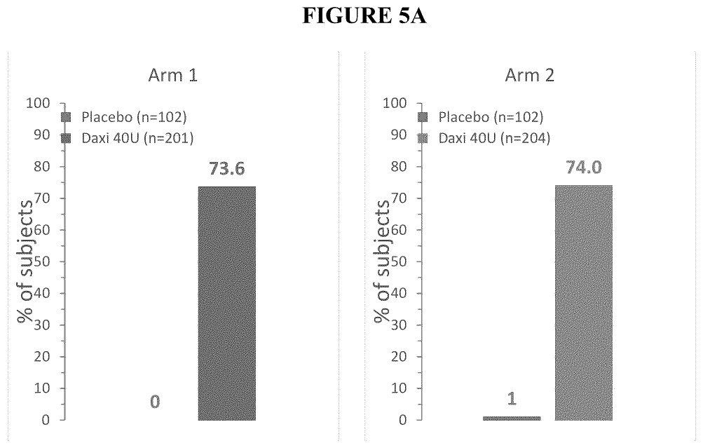

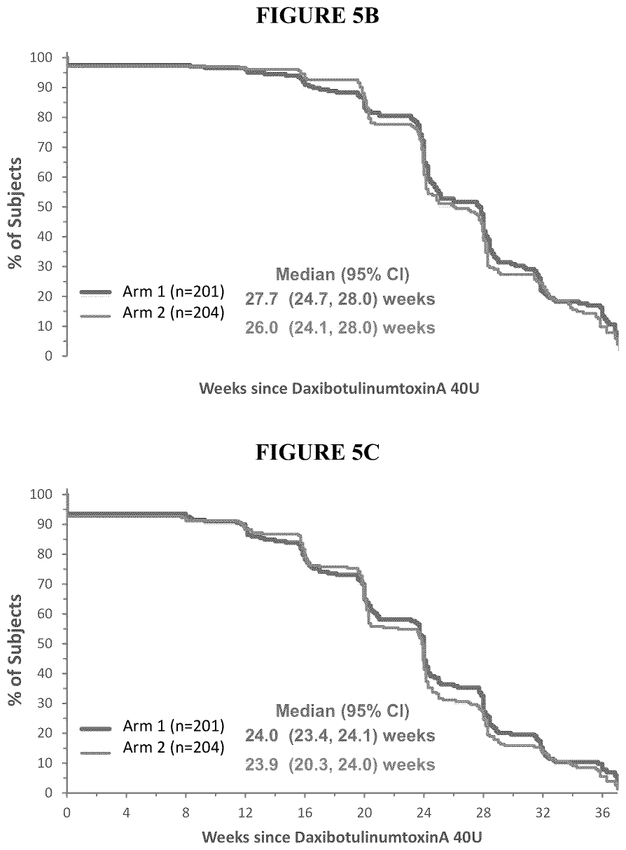

The Primary Endpoint was met with a 2-point composite response at Week 4 of 74% with RT002 versus 0% and 1% for placebo (p<0.0001) in Arms 1 and 2, respectively, with the results exceeding those observed in Example 5. Results are shown in FIG. 5A. Results using additional measures are shown in FIGS. 5B-5C.

FIG. 5B presents Kaplan-Meier Plots of Time to Return to, or Worse Than, Baseline on both IGA-FWS and PFWS Scales in patients in the RT002 group of Arm 1 and Arm 2 (Observed Cases) (ITT). Time to return to, or worse than, baseline on both IGA-FWS and PFWS is the number of days from treatment start date to the first visit at which both IGA-FWS and PFWS return to, or become worse than, baseline following the later of Weeks 1, 2, and 4, at which an improvement from baseline in both is observed. If no such visit is present, censoring occurs at the latest visit with both IGA-FWS and PFWS available. If there is no improvement from baseline in both IGA-FWS and PFWS at Weeks 1, 2, and 4, then time is set to 0.

FIG. 5C presents Kaplan-Meier Plots of Time of Maintenance of None or Mild Wrinkle Severity on either IGA-FWS or PFWS Scales in patients in the RT002 group of each of the two study arms (Observed Cases) (ITT). Time to return to moderate or severe on both IGA-FWS and PFWS is the number of days from treatment start date to the first visit at which either IGA-FWS or PFWS return to moderate or severe following the later of Weeks 1, 2, and 4 at which IGA-FWS or PFWS is none or mild. If no such visit is present, censoring occurs at the latest visit with either IGA-FWS or PFWS available. If neither IGA-FWS nor PFWS is none or mild at Weeks 1, 2, and 4, then time is set to 0.

Tables 19A-19B and Tables 20A-20B provide additional data regarding Time to Return analyses.

Tables 19A-19B present statistics for Return to, or Worse than Baseline on Both IGA-FWS and PFWS, for All Patients in the RT002 group of each of the two study arms at maximum frown (Observed Cases) (Intent-to-Treat Population). N is the number of patients in the RT002 Group. Percentages are based on N. Time to return to, or worse than, baseline on both IGA-FWS and PFWS is the number of days from treatment start date to the first visit at which both IGA-FWS and PFWS return to, or worse than, baseline following the later of Weeks 1, 2, and 4 at which an improvement from baseline in both is observed. If no such visit is present, censoring occurs at the latest visit with both IGA-FWS and PFWS available. If there is no improvement from baseline in both IGA-FWS and PFWS at Weeks 1, 2, and 4, then time is set to 0. Summary statistics are Kaplan-Meier estimates. + indicates a censored observation.

TABLE 19A

| | RT002 40 U |
|---|---|
| Statistic | N = 201 |
| Patients with the Event | 164 (81.6%) |
| Censored | 37 (18.4%) |
| Median Time to Response | 194.0 |
| 95% CI for Median | (173.0, 196.0) |
| Min-Max | 0, 263+ |
| Survival rate (95% CI) | |
| Week 8 | 97.5% ( 94.1%, 99.0%) |
| Week 12 | 96.0% ( 92.1%, 98.0%) |
| Week 16 | 90.9% ( 85.9%, 94.1%) |
| Week 20 | 83.1% ( 77.1%, 87.7%) |
| Week 24 | 64.8% ( 57.6%, 71.1%) |

TABLE 19B

| | RT002 40U |
|---|---|
| Statistic | N = 201 |
| Patients with the Event | 174 (85.3%) |
| Censored | 30 (14.7%) |
| Median Time to Response | 182.0 |
| 95% CI for Median | (169.0, 196.0) |
| Mm-Max | 0, 279 |
| Survival rate (95% CI) | |
| Week 8 | 97.1% (93.6%, 98.7%) |
| Week 12 | 96.1% (92.3%, 98.0%) |
| Week 16 | 93.1% (88.6%, 95.8%) |
| Week 20 | 85.6% (80.0%, 89.8%) |
| Week 24 | 60.7% (53.6%, 67.1%) |

Tables 20A-20B present Return to, or Worse than Baseline on Both IGA-FWS and PFWS, for Patients who met the 2-point Composite Response at Week 4 and are in the RT002 group of each of the two study arms at maximum frown (Observed Cases) (Intent-to-Treat Population). N is the number of patients in the RT002 Group where are 2-Point Composite Responders at Week 4. Percentages are based on N. Time to return to, or worse than, baseline on both IGA-FWS and PFWS is the number of days from treatment start date to the first visit at which both IGA-FWS and PFWS return to, or worse than, baseline following the later of Weeks 1, 2, and 4 at which an improvement from baseline in both is observed. If no such visit is present, censoring occurs at the latest visit with both IGA-FWS and PFWS available. If there is no improvement from baseline in both IGA-FWS and PFWS at Weeks 1, 2, and 4, then time is set to 0. Summary statistics are Kaplan-Meier estimates. + indicates a censored observation.

TABLE 20A

| | RT002 40 U |
|---|---|
| Statistic | N = 145 |
| Patients with the Event | 112 (77.2%) |
| Censored | 33 (22.8%) |
| Median Time for Response | 196.0 |
| 95% CI for Median | (182.0, 199.0) |
| Min-Max | 29+, 263+ |
| Survival rate (95% CI) | |
| Week 8 | 100.0% (100.0%, 100.0%) |
| Week 12 | 99.3% (95.1%, 99.9%) |
| Week 16 | 97.2% (92.6%, 98.9%) |
| Week 20 | 89.3% (82.9%, 93.4%) |
| Week 24 | 71.1% (62.8%, 77.9%) |

TABLE 20B

| | RT002 40 U |
|---|---|
| Statistic | N = 145 |
| Patients with the Event | 145 (96.7%) |
| Censored | 5 (3.3%) |
| Median Time for Response | 168.0 |
| 95% CI for Median | (156.0, 168.0) |
| Min-Max | 54, 279 |
| Survival rate (95% CI) | |
| Week 8 | 98.0% (93.9%, 99.4%) |
| Week 12 | 96.0% (91.3%, 98.2%) |
| Week 16 | 83.2% (76.2%, 88.4%) |
| Week 20 | 68.5% (60.4%, 75.3%) |
| Week 24 | 41.6% (33.7%, 49.4%) |

Tables 21A-21B provide results for percentage of different treatment groups showing none or mild wrinkles, based on IGA-FWS and PFWS over time, for each of the two arms of the study. *p<0.0001 vs placebo in both cases. Cochran-Mantel-Haensel test stratified by study center was used for response rate comparisons for Daxi (RT002) vs Placebo at each time point on ITT population. Missing data were imputed with worst post-baseline outcome for RT002 and best outcome for Placebo.

TABLE 21A
Investigator Assessment (IGA-FWS)

| | Arm 1 | | Arm 2 | |
|---|---|---|---|---|
| | RT002 40 U | Placebo | RT002 40 U | Placebo |
| Week | (n = 201) | (n = 102) | (n = 204) | (n = 102) |
| 2 | 93.5%* | 3.9% | 98.0%* | 2.0% |
| 4 | 97.5%* | 4.9% | 97.5%* | 3.9% |
| 8 | 91.5%* | 7.8% | 94.6%* | 2.9% |
| 12 | 84.1%* | 2.9% | 88.2%* | 2.9% |
| 16 | 71.1%* | 5.9% | 74.0%* | 2.9% |
| 20 | 53.2% | 2.9% | 54.4%* | 2.9% |
| 24 | 35.3%* | 2.0% | 29.4%* | 2.0% |

TABLE 21B
Patient Assessment (PFWS)

| | Arm 1 | | Arm 2 | |
|---|---|---|---|---|
| | RT002 40 U | Placebo | RT002 40 | Placebo |
| Week | (n = 201) | (n = 102) | U (n = 204) | (n = 102) |
| 2 | 93.5%* | 3.9% | 98.0%* | 2.0% |
| 4 | 97.5%* | 4.9% | 97.5%* | 3.9% |
| 8 | 92.5%* | 3.9% | 91.2%* | 3.9% |
| 12 | 92.0% | 1.0% | 90.2%* | 3.9% |
| 16 | 84.6%* | 2.0% | 85.3%* | 6.9% |
| 20 | 72.6%* | 2.9% | 71.6%* | 5.9% |
| 24 | 57.2%* | 5.9% | 53.4%* | 5.9% |

Tables 22A-22B present Patient Global Satisfaction with treatment at the Week 4 visit (Observed cases) (Intent-to-Treat Population), for patients in each of the two study arms.

TABLE 22A

| | | | |
|---|---|---|---|
| Patient Global Satisfaction | Placebo | RT002 40 U | Difference |
| with Treatment | N = 102 | N-201 | (90% CI) |

Week 4, Number of
Patients at Scale Level (%)

| | | | |
|---|---|---|---|
| Very Dissatisfied | 38 (37.3%) | 2 (1.0%) | 4.2 (4.0, 4.4) |
| Dissatisfied | 25 (24.5%) | 1 (0.5%) | |
| Somewhat Dissatisfied | 7 (6.9%) | 1 (0.5%) | |
| Neither Satisfied Nor | 23 (22.5%) | 5 (2.5%) | |
| Dissatisfied | | | |
| Somewhat Satisfied | 2 (2.0%) | 11 (5.5%) | |
| Satisfied | 2 (2.0%) | 40 (19.9%) | |
| Very Satisfied | 0 | 136 (67.7%) | |

TABLE 22B

| | | | |
|---|---|---|---|
| Patient Global Satisfaction | Placebo | RT002 40 U | Difference |
| with Treatment | N = 102 | N-201 | (90% CI) |

Week 4, Number of
Patients at Scale Level (%)

| | | | |
|---|---|---|---|
| Very Dissatisfied | 38 (37.3%) | 2 (1.0%) | 4.2 (4.0, 4.4) |
| Dissatisfied | 25 (24.5%) | 1 (0.5%) | |
| Somewhat Dissatisfied | 7 (6.9%) | 1 (0.5%) | |
| Neither Satisfied Nor | 23 (22.5%) | 5 (2.5%) | |
| Dissatisfied | | | |
| Somewhat Satisfied | 2 (2.0%) | 11 (5.5%) | |
| Satisfied | 2 (2.0%) | 40 (19.9%) | |
| Very Satisfied | 0 | 136 (67.7%) | |

Tables 23A-23B provide results for percentage of different treatment groups showing none or mild wrinkles, based on IGA-FWS and PFWS, over time, for each of the two arms of the study compared to the results in the Example 5 study. (*p<0.0001 vs placebo in both cases; **p<0.01 (vs Placebo in the study of Example 5). Cochran-Mantel-Haensel test stratified by study center was used for response rate comparisons for Daxi (RT002) vs Placebo at each time point on ITT population. Missing data were imputed with worst post-baseline outcome for RT002 and best outcome for Placebo.

TABLE 23A

| | Arm 1 | Arm 2 | Example 5 |
|---|---|---|---|
| | RT002 40 U | RT002 40 U | RT002 40 U |
| Week | (n = 201) | (n = 204) | (n = 39) |
| 2 | 93.5%* | 98.0%* | 97.3%** |
| 4 | 97.5%* | 97.5%* | 97.4%** |
| 8 | 91.5%* | 94.6%* | 97.4%** |
| 12 | 84.1%* | 88.2%* | 84.6%** |
| 16 | 71.1%* | 74.0%* | 66.7%** |
| 20 | 53.2% | 54.4%* | 46.2%** |
| 24 | 35.3%* | 29.4%* | 30.8%** |

TABLE 23B

| | Arm 1 | Arm 2 | Example 5 |
|---|---|---|---|
| | RT002 40 U | RT002 40 U | RT002 40 U |
| Week | (n = 201) | (n = 204) | (n = 39) |
| 2 | 92.5%* | 91.2%* | 91.9%** |
| 4 | 92.0%* | 90.2%* | 94.9%** |
| 8 | 84.6%* | 85.3%* | 86.8%** |
| 12 | 72.6%* | 71.6%* | 84.6%** |
| 16 | 57.2%* | 53.4%* | 61.5%** |
| 20 | 44.8% | 35.8%* | 35.9%** |
| 24 | 23.9%* | 21.6%* | 20.5%** |

Tables 24A-24B further show that robust None or Mild response rates were observed through Week 24 on Investigator Assessment (IGA-FWS) (Table 24A) and on Patient Assessment (PFWS) (Table 24B) (*p<0.0001 vs. placebo at all-time points through Week 24; Cochran-Mantel-Haenszel test stratified by study center was used for response rate comparison for RT002 vs Placebo at each time point on ITT population; missing data were imputed with worst post-baseline outcome for RT002 and best outcome for Placebo). For example, Arm 1 and Arm 2 had response rates of 71% and 74% at Week 16, and 35% and 29% at Week 24 for IGA-FWS, respectively.

TABLE 24A

| | Arm 1 | | Arm 2 | |
|---|---|---|---|---|
| | RT002 40 U | Placebo | RT002 40 | Placebo |
| Week | (n = 201) | (n = 102) | U (n = 204) | (n = 102) |
| 2 | 93.5%* | 3.9% | 98.0% | 2.0% |
| 4 | 97.5%* | 4.9% | 97.5%* | 3.9% |
| 8 | 91.5%* | 7.8% | 94.6%* | 2.9% |
| 12 | 84.1%* | 2.9% | 88.2%* | 2.9% |
| 16 | 71.1%* | 5.9% | 74.0%* | 2.9% |
| 20 | 53.2%* | 2.9% | 54.4%* | 2.9% |
| 24 | 35.3%* | 2.0% | 29.4%* | 2.0% |

TABLE 24B

| | Arm 1 | | Arm 2 | |
|---|---|---|---|---|
| | RT002 | | RT002 | |
| | 40 U | Placebo | 40 U | Placebo |
| Wk | n = 201 | n = 102 | n = 204 | n = 102 |
| 2 | 92.5%* | 3.9% | 91.2%* | 3.9% |
| 4 | 92.0%* | 1.0% | 90.2%* | 3.9% |
| 8 | 84.6%* | 2.0% | 85.3%* | 6.9% |
| 12 | 72.6%* | 2.9% | 71.6%* | 5.9% |
| 16 | 57.2%* | 5.9% | 53.4%* | 5.9% |
| 20 | 44.8%* | 2.9% | 35.8%* | 6.9% |
| 24 | 23.9%* | 1.0% | 21.6%* | 2.0% |

Tables 25A-25B compare these results with those from Example 5, and show robust None or Mild response rates observed through Week 24 on Investigator Assessment (IGA-FWS) (Table 25A) and on Patient Assessment (PFWS) (Table 25B) (*p<0.0001 (vs Placebo), **p<0.01 (vs Placebo); Cohran-Mantel-Haenszel test stratified by study center was used for response rate comparison for RT002 vs Placebo at each time point on ITT population; missing data were imputed with worst post-baseline outcome for RT002 and best outcome for Placebo). There was robust None or Mild response rates observed on IGA-FWS through Week 24. The response rates for each of the two arms were 71% and 74%, at Week 16, and 35%; and 29%, at Week 24, respectively (p<0.0001 vs. placebo at all-time points through Week 24). In Example 5's study, the RT002 40 U dose response rate was 67% at Week 16, and 31% at Week 24, compared with OnabotulinumtoxinA 20 U Week 16 response rate of 31.7% and Week 24 response rate of 11.9% (see again Carruthers 2017; and Allergan, Inc. BOTOX® (onabotulinumtoxinA) Prescribing Information, 2013).

TABLE 25A

| | Arm 1 | Arm 2 | EX. 5 |
|---|---|---|---|
| | RT002 | RT002 | RT002 |
| | 40 U | 40 U | 40 U |
| Wk | n = 201 | n = 204 | n = 39 |
| 2 | 93.5%* | 98.0%* | 97.3%** |
| 4 | 97.5%* | 97.5%* | 97.4%** |
| 8 | 91.5%* | 94.6%* | 97.4%** |
| 12 | 84.1%* | 88.2%* | 84.6%** |
| 16 | 71.1%* | 74.0%* | 66.7%** |
| 20 | 53.2%* | 54.4%* | 46.2%** |
| 24 | 35.3%* | 29.4%* | 30.8%** |

TABLE 25B

| | Arm 1 | Arm 2 | EX. 5 |
|---|---|---|---|
| | RT002 | RT002 | RT002 |
| | 40 U | 40 U | 40 U |
| Wk | n = 201 | n = 204 | n = 39 |
| 2 | 92.5%* | 91.2%* | 91.9%** |
| 4 | 92.0%* | 90.2%* | 94.9%** |
| 8 | 84.6%* | 85.3%* | 86.8** |
| 12 | 72.6%* | 71.6%* | 84.6%** |
| 16 | 57.2%* | 53.4%* | 61.5%** |
| 20 | 44.8% | 35.8%* | 35.9%** |
| 24 | 23.9%* | 21.6%* | 20.5%** |

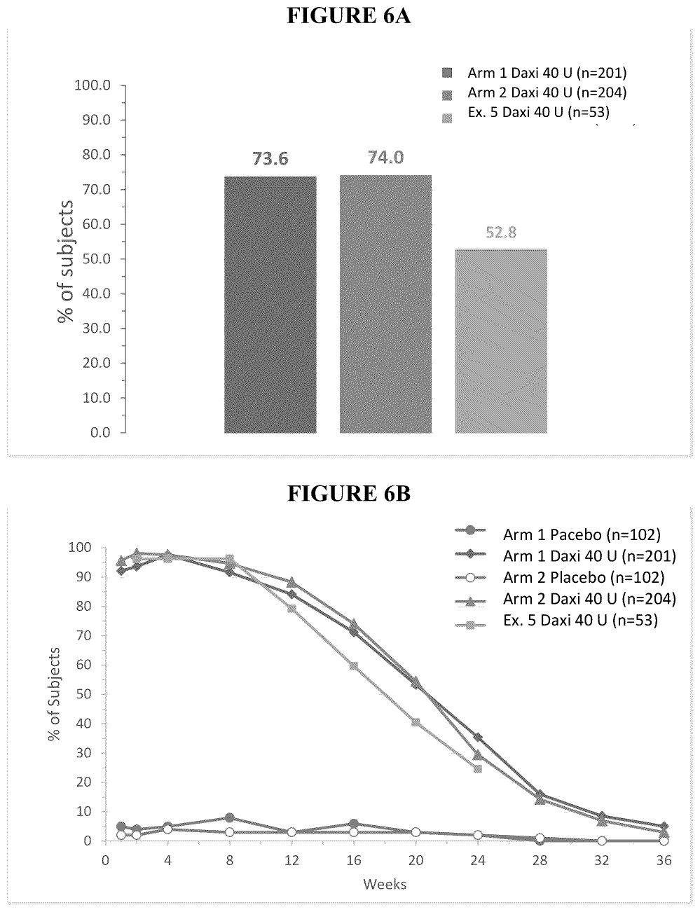

As also showing in FIGS. 6A-6B, these results exceed those obtained in Example 5 that showed, at Week 4, a 2-point composite response of 52.8% with RT002 (FIG. 6A) (*p<0.0001 (vs Placebo on a Cochran-Mantel-Haenszel test stratified by study center) (see also Carruthers, 2017).

FIG. 6B compares Arm 1 and Arm 2 of this study to Example 5, in terms of the none or mild response on IGA-FWS and PFWS over time. Example 5 showed, at Week 16, a response of 67%, and at Week 24, a response of 31% (FIG. 6B). In the Phase 3 study, missing data were imputed with the worst post-baseline outcome (or best outcome for Placebo arm) on visits up to Week 24. Non-responder imputation was used for visits post Week 24. In Example 5, response rates were of subjects with data.

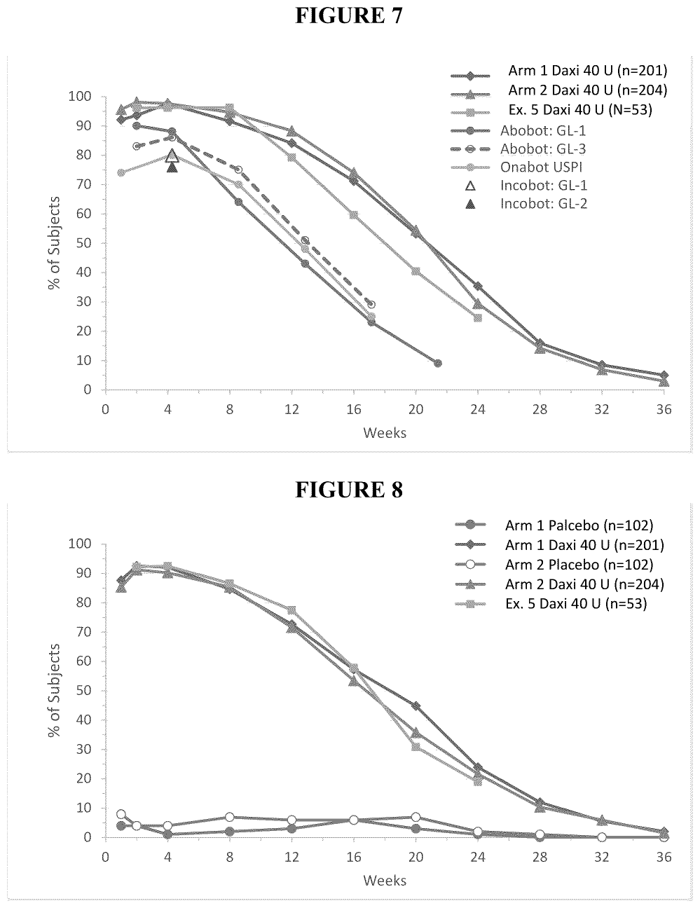

FIG. 7 presents percent of subjects maintaining none or a mild wrinkles based on IGA-FWS and PFWS score over time, for each of the two arms of the present study, for Example 5, and for various other botulinum toxin formulations. FIG. 7 builds upon FIG. 6 by including the none or mild response per IGA-FWS and PFWS over time per the package inserts of onabotulinumtoxinA, abobotulinumtoxinA, and incobotulinumtoxinA. For example, OnabotulinumtoxinA 20 U, that showed a Week 16 response rate of 31.7% and a Week 24 response rate of 11.9%. Specifically, the bright blue solid line represents Abobot GL-1 (AbobotulinumtoxinA described in Dysport's US Package insert for their GL-1 study); the bright blue, dashed line represents Abobot GL-3 (abobotulinumtoxinA described in Dysport's US Package insert for their GL-3 study), the orange represent Onabot USP1 (OnabotulinumtoxinA as describing in the corresponding US Package insert); the red open triangle represents Incobot GL-1 (IncobotulinumtoxinA described in Xeomin's US Package insert for their GL-1 study); and the red closed triangle represents Incobot GL-2 (IncobotulinumtoxinA described in Xeomin's US Package insert for their GL-2 study). In the Phase 3 study arms, for the ITT population, missing data were imputed with the worst post-baseline outcome (or best outcome for Placebo arm) on visits up to Week 24. Non-responder imputation was used for visits post Week 24. With respect to the results of Example 5, response rates were of ITT subjects with data.

FIGS. 8-10 further present results of the Phase 3 study. FIG. 8 depicts none or mild response per PFWS relative to Example 5. In the Phase 3 study, missing data were imputed with the worst post-baseline outcome (or best outcome for Placebo arm) on visits up to Week 24. Non-responder imputation was used for visits post Week 24. In Example 5, response rates were of subjects with data.

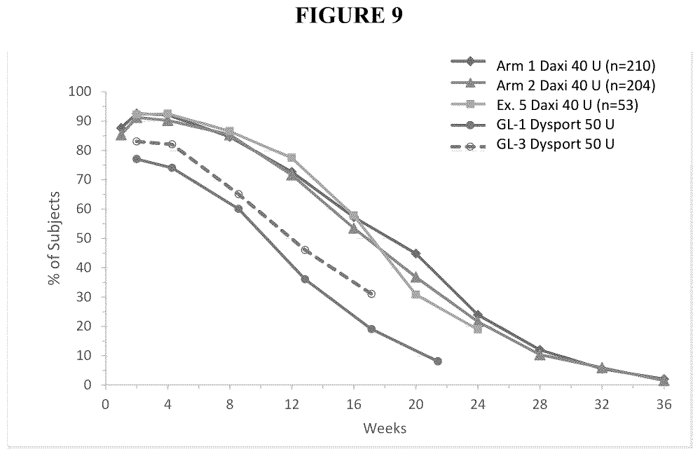

FIG. 9 depicts none or mild response per PFWS relative to abobotulinumtoxinA. In the Phase 3 study (ITT), missing data were imputed with the worst post-baseline outcome (or best outcome for Placebo arm) on visits up to Week 24. Non-responder imputation was used for visits post Week 24. In Example 5, response rates were of ITT subjects with data.

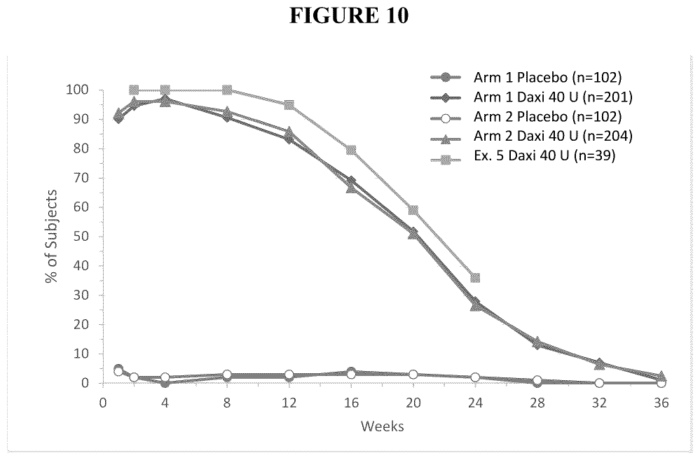

FIG. 10 depicts the rate of 2:1 point response on IGA-FWS over time of Arm 1 and Arm 2 (ITT) of the present study relative to Example 5 (PP). In the Phase 3 study, missing data were imputed with the worst post-baseline outcome (or best outcome for Placebo arm) on visits up to Week 24. Non-responder imputation was used for visits post Week 24. In Example 5, response rates were of subjects in PP population.

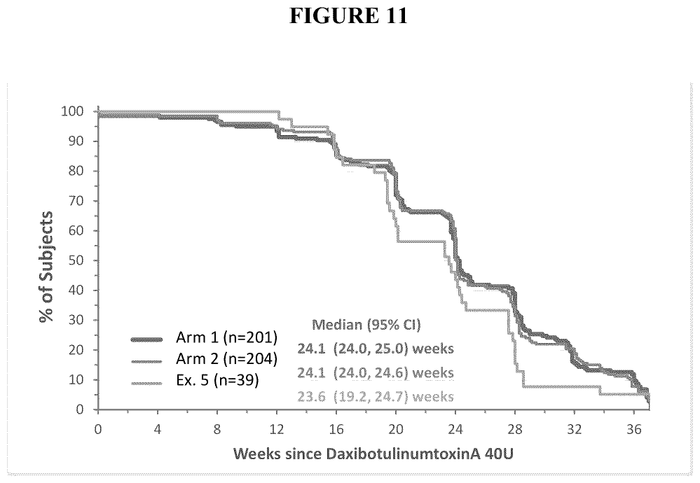

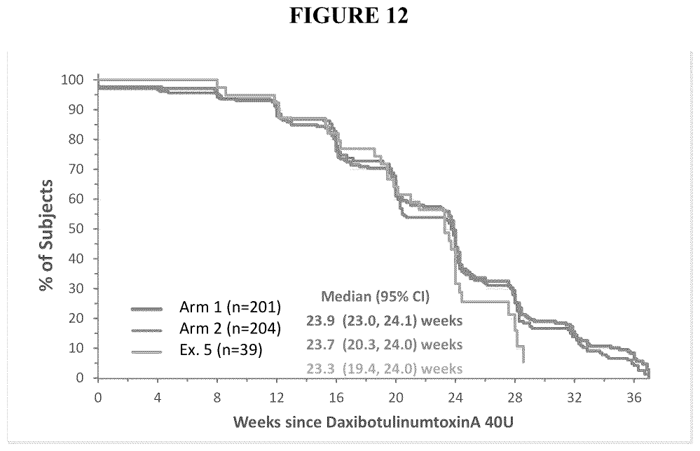

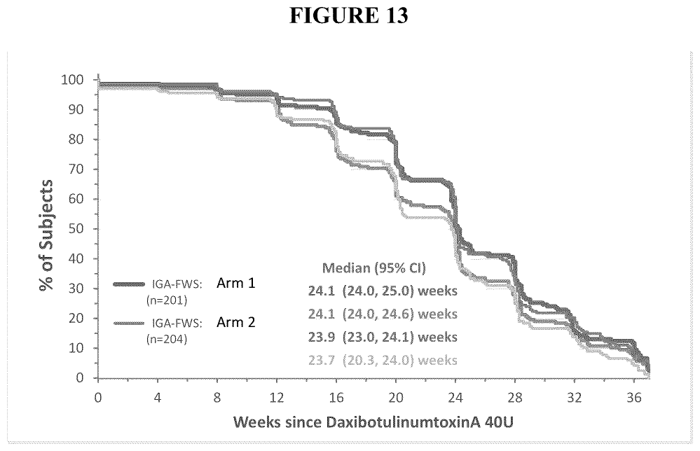

FIGS. 11-13 further summarize these results. The two arms of this study are the first and only Phase 3 trials in patients with moderate to severe glabellar lines that demonstrated confirmatory efficacy of 2:24 weeks on multiple clinically meaningful outcome measures. Both Phase 3 studies exceeded Example 5's study results, with median duration of 2:1-point improvement from baseline on IGA-FWS and PFWS of 24.1 and 23.7-23.9 Weeks on both investigator (FIG. 11) and subject assessments (FIG. 12), respectively. By contrast, the study in Example 5 demonstrated a 2:1-point improvement on IGA-FWS of 23.6 weeks median duration with RT002 40 U vs. 18.8 weeks with onabotulinumtoxinA 20 U (p<0.03) (Carruthers, 2017). FIG. 13 depicts a comparison of IGA-FWS and PFWS for the two arms of this study.

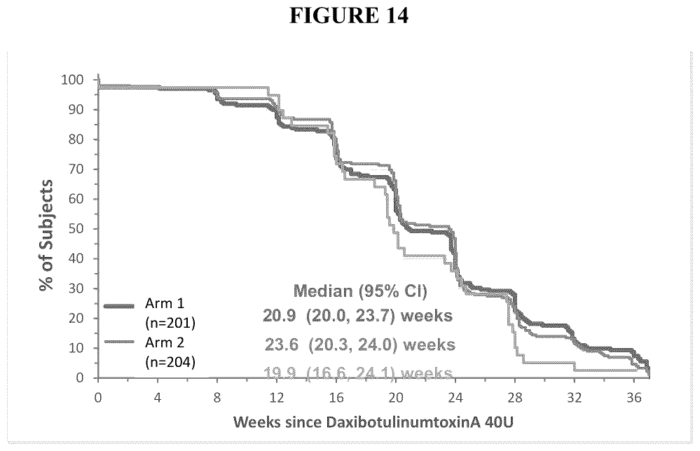

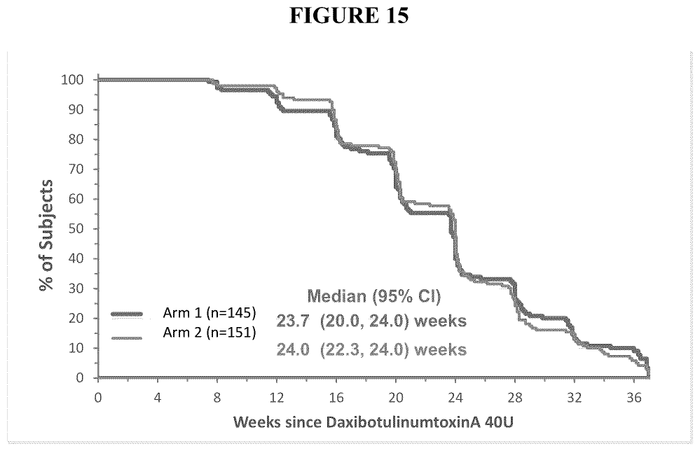

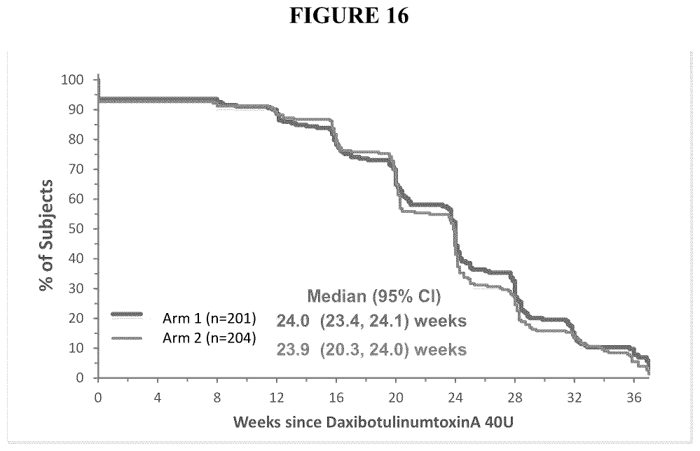

FIGS. 14-16 depict that maintenance of none or mild wrinkle severity status (Score of 0 or 1) with 40 U RT002 on IGA-FWS and PFWS replicated the duration observed with 2:1 point improvement results. FIG. 14 presents percent of subjects maintaining none or mild wrinkle severity status (Score of 0 or 1) on IGA-FWS over time, for each of the two arms of the present study (purple and blue lines) and for Example 5 (red line). FIG. 15 presents percent of subjects maintaining none or mild wrinkle severity status (Score of 0 or 1) on IGA-FWS over time, for each of the two arms of the present study (purple and blue lines), based on 2-point composite responders at Week 4. FIG. 16 presents percent of subjects maintaining none or mild wrinkle severity status (Score of 0 or 1) on either IGA-FWS or PFWS over time, for each of the two arms of the present study (purple and blue lines).

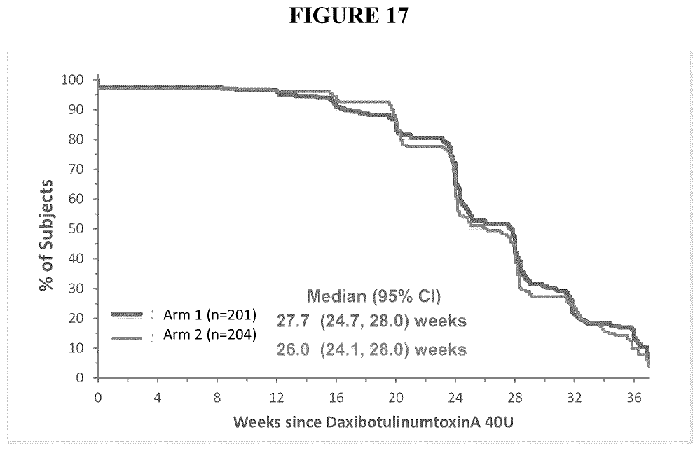

FIG. 17 depicts mean duration of 27.7 weeks (6.5 months) and 26 weeks (6.1 months) in Arm 1 and 2, respectively, of this study. Median time to return to baseline wrinkle severity with RT002 on both IGA-FWS and PFWS exceeded six months.

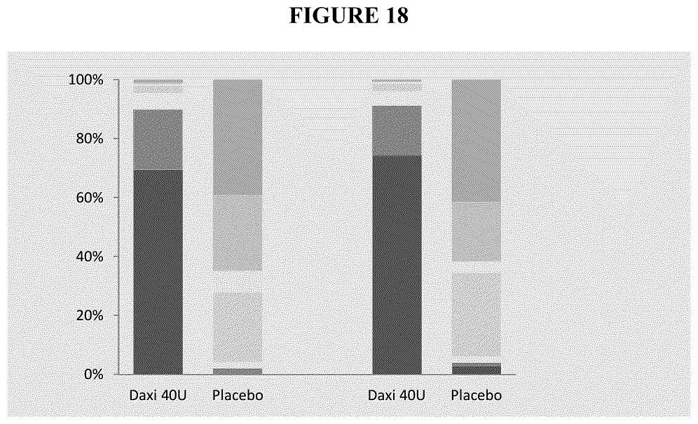

Patient Global Satisfaction: High patient global satisfaction rates were observed at Week 4 across both arms of the study. FIG. 18 depicts satisfaction rates of ‘Satisfied’ or ‘Very Satisfied’ of 91% and 88% for Arms 1 and 2, respectively, on a 7-point scale.

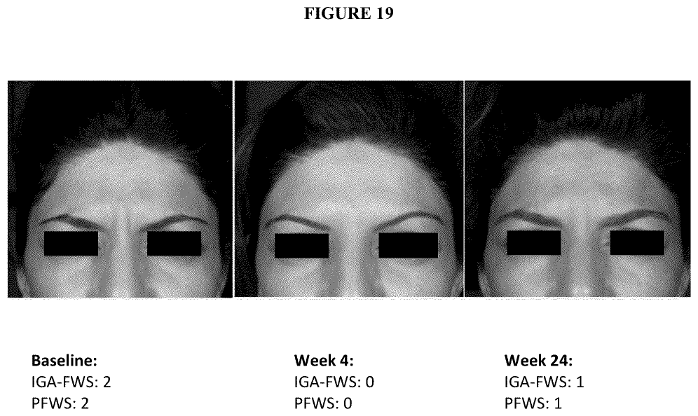

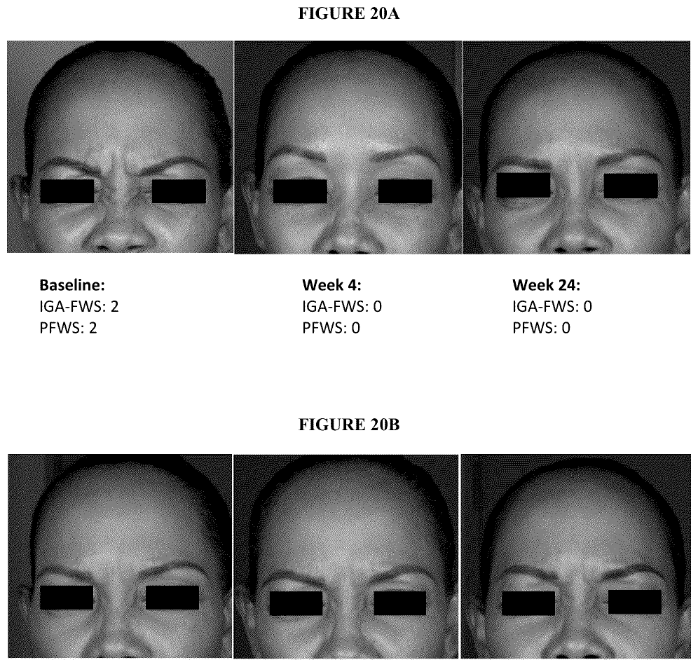

FIGS. 19-20 show photographs of exemplary subjects of the Phase 3 study. FIG. 19 presents photographs of a subject showing 2-point improvement by IGA-FWS and PFWS at Week 4 (maximum frown) after RT002 40 U treatment; and a 1-point sustained duration of effect through Week 24.

FIGS. 20A-20B presents photographs of a different subject showing 2-point improvement by IGA-FWS and PFWS at Week 4 (maximum frown) after RT002 40 U treatment; and a 1-point sustained duration of effect through Week 24s (FIG. 20B) and 32 weeks (FIG. 20B).

### Safety:

RT002 40U was observed to be generally safe and well-tolerated through Week 36 in both arms. Percentage of subjects in Arm I and 2 with adverse events were 36% and 46%, respectively, in the RT002 group; and 25% and 24%, respectively, in the placebo group. See Table 26, summarizing adverse events. Majority of events in the RT002 group were mild in severity and considered to be unrelated to study drug. No subjects in the RT002 group discontinued secondary to AEs. Two subjects in each arm experienced a Serious AE in the RT002 group, neither of which were treatment related. Treatment related AEs in Arm I and 2 occurred in 17.4% and 21.0% of subjects, respectively, in the RT002 group; and 8% and 9%, respectively, in the placebo group.

TABLE 26

| | Arm 1 | | Arm 2 | |
|---|---|---|---|---|
| | | RT002 | | RT002 |
| | Placebo | 40 U | Placebo | 40 U |
| | (n = 102) | (n = 201) | (n = 101) | (n = 204) |
| Death, n (%) | 0 | 0 | 0 | 0 |
| Serious AEs (SAE), | 1 (1.0%) | 2 (1.0%) | 1 (1.0%) | 2 (1.0%) |
| n (%) | | | | |
| Subjects with any | 25 (24.5%) | 72 (35.8%) | 24 (23.8%) | 94 (45.9%) |
| AE, n (%) | | | | |
| Severe | 1 (1.0%) | 4 (2.0%) | 1 (1.0%) | 2 (1.0%) |
| Subjects with any | 8 (7.8%) | 35 (17.4%) | 10 (9.9%) | 43 (21.0%) |
| treatment- | | | | |
| related* AE, n (%) | | | | |
| SAE | 0 | 0 | 0 | 0 |
| Subjects with any | 0 | 0 | 0 | 0 |
| AE leading to Study | | | | |
| Discontinuation, | | | | |
| n (%) | | | | |

In the RT02 group across both studies, the most common events were headache (6-7%) and injection site pain (2.4%-5%); rates of injection-related edema and erythema were: 2.4% and all cases were mild in severity. Overall eyelid ptosis rate of 2.2% was observed, with a mean duration of 60 days. Table 27 summarizes the number of Treatment-Related Adverse Events by Preferred Term (>2% in any Arm) (*6 cases of mild severity, 3 cases of moderate severity; all resolved without sequelae. Mean duration 60 days; t Ocular Disorder with >2 cases per group. All cases mild in severity. t Mean duration of 30 days).

TABLE 27

| | Arm 1 | | Arm 2 | |
|---|---|---|---|---|
| | | RT002 | | RT002 |
| | Placebo | 40 U | Placebo | 40 U |
| Preferred Term | (n = 102) | (n = 201) | (n = 101) | (n = 205) |
| Headache | 3 (2.9%) | 4 (7.0%) | 1 (1.0%) | 12 (5.9%) |
| Injection site pain | 4 (3.9%) | 10 (5.0%) | 4 (4.0%) | 5 (2.4%) |
| Eyelid ptosis* | 0 | 5 (2.5%) | 0 | 4 (2.0%) |
| Injection site erythema | 0 | 0 | 4 (4.0%) | 5 (2.4%) |
| Injection site oedema | 0 | 1 (0.5%) | 3 (3.0%) | 5 (2.4%) |
| Blepharospasm | 0 | 0 | 0 | 3 (1.5%) |
| Brow ptosis | 0 | 2 (1.0%) | 0 | 1 (0.5%) |

Severe and serious adverse events are presented in Table 28 and Table 29. Table 28 summarizes Severe Adverse Events** by Preferred Term (*The severe AE is also a serious AE; ** none of the Severe Adverse Events were treatment related).

TABLE 28

| | Arm 1 | | Arm 2 | |
|---|---|---|---|---|
| | | RT002 | | RT002 |
| Severe AE | Placebo | 40 U | Placebo | 40 U |
| Preferred Term | (n = 102) | (n = 201) | (n = 101) | (n = 205) |
| Bone marrow failure* | 0 | 1 (0.5%) | 0 | 0 |
| Influenza | 0 | 1 (0.5%) | 0 | 0 |
| Sepsis* | 0 | 1 (0.5%) | 0 | 0 |
| Carpal tunnel | 0 | 1 (0.5%) | 0 | 0 |
| syndrome | | | | |
| Anxiety* | 1 (1.0%) | 0 | 0 | 0 |
| Nephrolithiasis | 0 | 1 (0.5%) | 0 | 0 |
| Respiratory tract | 0 | 1 (0.5%) | 0 | 0 |
| congestion | | | | |
| Conjunctivitis | 0 | 0 | 0 | 1 (0.5%) |
| Uterine perforation* | 0 | 0 | 0 | 1 (0.5%) |
| Leiomyosarcoma | 0 | 0 | 1 (1.0%) | 0 |
| recurrent* | | | | |
| Uterine leiomyoma* | 0 | 0 | 0 | 1 (0.5%) |

Table 29 summarizes Serious Adverse Events** by Preferred Term (*none of the Serious AE's above are related to treatment).

TABLE 29

| | Arm 1 | | Arm 2 | |
|---|---|---|---|---|
| | | RT002 | | RT002 |
| | Placebo | 40 U | Placebo | 40 U |
| Preferred Term | (n = 102) | (n = 201) | (n = 101) | (n = 205) |
| Bone marrow failure* | 0 | 1 (0.5%) | 0 | 0 |
| Sepsis | 0 | 1 (0.5%) | 0 | 0 |
| Anxiety* | 1 (1.0%) | 0 | 0 | 0 |
| Uterine perforation* | 0 | 0 | 0 | 1 (0.5%) |
| Leiomyosarcoma | 0 | 0 | 1 (1.0%) | 0 |
| recurrent* | | | | |
| Uterine leiomyoma* | 0 | 0 | 0 | 1 (0.5%) |
| Bone marrow failure* | 0 | 1 (0.5%) | 0 | 0 |
| Sepsis | 0 | 1 (0.5%) | 0 | 0 |

### (i) Example 8: Follow up Safety Study on Injectable Botulinum Toxin Formulation Showing Improved Safety, Higher Responder Rates, and Longer Duration of Effect in Treating Glabellar Lines Over Successive Treatments

In addition to the two planned pivotal trials, the Phase 3 program includes a long-term, open-label safety trial, which is designed to evaluate the long-term safety of RT002 for the treatment of moderate to severe glabellar lines in adults following both single and repeat treatment administration. The long-term safety trial is expected to enroll approximately 1,500 subjects at multiple sites. Depending on the number of treatments and duration of follow-up, a subject may be in trial for a maximum of 84 weeks.

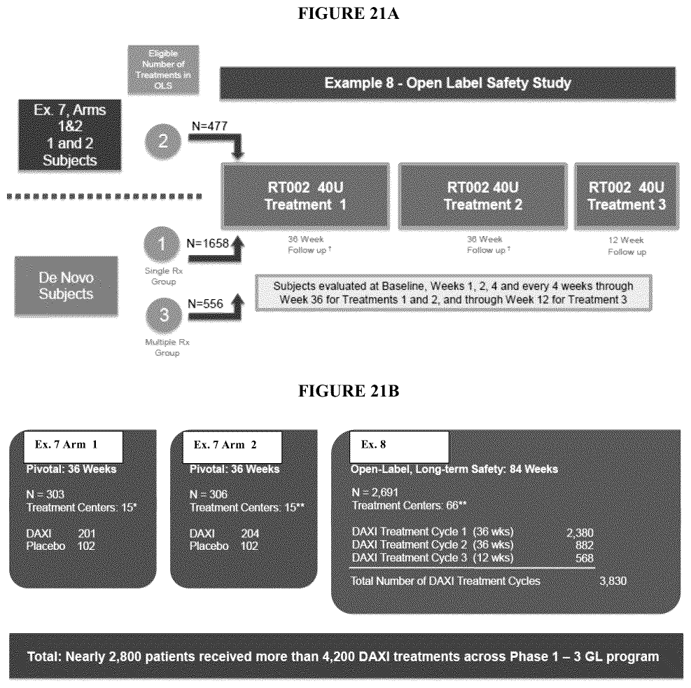

The safety study involved a Phase 3 prospective, open-label, multi-center, repeat-dose trial to assess the safety of single and repeat administrations of RT002 in treating moderate to severe glabellar lines. The safety study included about 60 centers and approximately 1,500 subjects enrolled, in addition to approximately 600 roll-over subjects from the phase 3 arms 1 and 2 of Example 7, described herein, for repeat treatments. Subjects received up to three repeat treatments. Subjects from Example 7 received one treatment in the parent trial and up to two in this trial, for a total of three treatments; newly-enrolled subjects received up to two treatments. FIG. 21A depicts the study design. The follow ups refer to subjects eligible for re-treatment beginning at Week 12 when both IGA-FWS and PFWS return to baseline. FIG. 21B depicts an overview of this study compared with Example 7's Arms 1 and 2. * indicates US centers; ** indicates US and Canadian centers.

All subjects were followed for at least 12 weeks, and up to 36 weeks, after each treatment for safety assessments. If both PFWS and IGA-FWS scores at maximum frown return to baseline at the Week 12 visit, or at a visit between Weeks 12 and 36, the visit at which these two scores are recorded became the Final Evaluation Visit, and for those subjects who received multiple treatments, this visit served as a re-treatment visit and provided the baseline for this trial; also, these subjects had a Final Evaluation Visit at Week 12 following their final eligible treatment.

Dosage Regimen: All treatments were intramuscular injections administered by a trained physician. Administration was performed as described in Example 7. See also FIG. 23, indicating injection sites used in this trial. Subjects received total of 0.5 mL of treatment, with 0.1 mL administered per injection to five injection sites: two injections into each corrugator muscle and one injection in the procerus muscle. Dosage was 40 U per injection treatment, divided among the five injection sites. The duration of subject participation varied depending on number of treatments, response to RT002, and duration of follow-up. A subject may have been on trial for a maximum of 86 weeks, inclusive of a two week screening period.

Trial Visits: Screening (−Week 2), Treatment (Day 0), post the first treatment follow-up at Weeks 1, 2, 4, 8, and 12, then at Weeks 16, 20, 24, 28, 32, 36 or until the re-treatment. Post the second treatment follow-up (for the subjects who are re-treated), at Weeks 1, 2, 4, 8, and 12, then at Weeks 16, 20, 24, 28, 32, and 36 or until the re-treatment (3^{rd} dose). Post the third treatment follow-up (for the subjects who receive the 3^{rd} treatment), at Weeks 1, 2, 4, 8, and 12. Subjects eligible for re-treatment showed return of IGA-FWS and PFWS severity scores to baseline. Allowed variation from scheduled visit day were +/−2 days for Week 1 (post treatment); +/−3 days for each of Weeks 2, 4, 8, 12, 16, 20, 24, 28, 32, and 36.

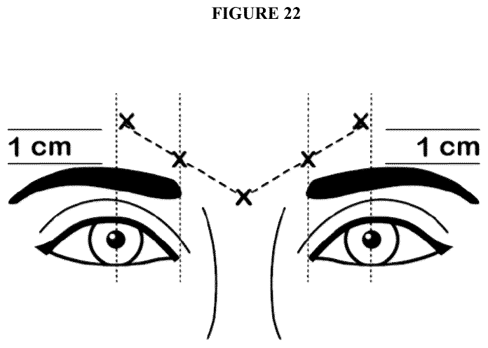

FIG. 22 further depicts the schedule of trial assessments followed in this trial. In particular, “a” notes Final Evaluation visit of Example 7 (Phase 3 study, arms 1 and 2) end of trial PT, and Week 12 serum antibody test, served as the baseline for the roll overs in this trial; “b” notes that subjects may be eligible for retreatment when the IGA-FWS and PFWS returned to baseline at Week 12, or at a visit between Weeks 12 and 36; “c” notes that subjects who have received multiple treatments completed the Final Evaluation Visit at Week 12 following the final treatment; “d” notes procedure performed pre-treatment; “e” notes procedure performed post-treatment; “e” notes that, if positive, this test was confirmed by serum pregnancy test; “g” notes that PT was only collected at screening; “h” notes that, if signs or symptoms of distant spread of toxin were reported, vital signs were recorded; “i” notes that photographs included the subject's frontal view at maximum frown and at rest after maximum frown. During the first treatment, these photos were taken at baseline, Weeks 4, 16, 20, 24, and EOS. During the second treatment, photos were taken at baseline, Week 4, and EOS. At select centers, photographs also included the subject's forehead at rest and then at maximum forehead raise at baseline and all Follow-up Visits; “j” notes that the photographs were obtained of the subject in primary gaze with brow relaxed and in primary gaze with brow elevated; “k” notes that, if signs of ptosis were reported or observed, photographs were obtained.

Safety Evaluations: clinical laboratory tests (hematology, chemistry, prothrombin time [PT], urinalysis); urine pregnancy test (UPT) for women of child-bearing potential (WOCBP); serum antibody tests for RT002 and RTP004; injection site evaluation; cranial nerves II-VII assessment; evaluation of facial muscle strength; concomitant medications; collection of adverse events (AES); distant spread of toxin query; vital signs; and physical examination.

As outlined in Table 30, non-fasting samples for hematology, chemistry, coagulation (prothrombin time; screening only) and urinalysis were collected at Screening, Week 4 visits, prior to re-treatment (as applicable), and at the Final Evaluation Visit. At Screening and Week 2, 4, and 12 Visits, blood samples for antibodies were collected.

TABLE 30
Clinical Laboratory Tests

| Serum | | | |
|---|---|---|---|
| Chemistry | Hematology | Urinalysis | Additional Tests |
| Glucose Total bilirubin | Hemoglobin | Specific | Prothrombin time (PT) |
| Alkaline phosphatasem Blood urea | Hematocrit | Gravity pH | (screening Urine Pregnancy |
| nitrogen Alanine aminotransferase | Red Blood Cell | Glucose | (WOCBP* only) |
| Aspartate | Count | Protein | Serum antibodies for |
| aminotransferase | Platelet Count | Blood | daxibotulinumtoxinA and |
| | Leukocyte Count | Bilirubin | |
| | (total) | Ketones | |
| | Leukocyte Count | | |
| | (differential) | | |

WOCBP = Women of child-bearing potential
*Screening Week 2, 4, and 12 visits only

Antibody Testing: If antibody titer developed to the product during the course of RT002 treatment, samples from that subject were subjected to mouse protection assay to test for neutralizing antibody.

Serum antibodies for RT002 and RTP004 were summarized using descriptive statistics, and trends analyzed for positive antibody results and correlated to clinically significant events related to immunogenicity. Specifically, descriptive statistics were presented showing the frequency of positive antibody results as well as associated clinically significant events related to immunogenicity over time.

Vital Signs: Vital signs (i.e., body temperature, respiration rate, sitting radial pulse rate, and sitting systolic and diastolic blood pressures) were obtained at the Screening and Treatment Visit (pre- and post-treatment), Week 2, Final Evaluation or Early Discontinuation Visits and at any visit where signs or symptoms of botulinum toxicity were reported.

Physical Examination: A physical examination, in addition to vital signs, including neurological examination of the face, general appearance, skin, neck (including thyroid), eyes, ears, nose, throat, heart, lungs, abdomen, lymph nodes, and extremities was conducted at Screening, Week 2 and Final Evaluation or Early Discontinuation Visits. Significant physical examination findings that are present prior to investigational product administration were included as medical history. Significant physical examination findings which meet the definition of an adverse event were recorded.

Injection Site Evaluation: Injection sites were evaluated at the Screening Visit, Treatment Visit pre- and post-treatment, Follow-up Visits, and Final Evaluation Visit or Early Discontinuation Visit, if applicable. The assessment was done as a global evaluation of the 5 injection sites, as shown in Table 11, above.

Assessment of Cranial Nerves II-VII: Evaluation of cranial nerves II-VII (left and right sides separately) was performed at Screening, Treatment Visit pre- and post-treatment, Follow-up Visits and Final Evaluation Visit or Early Discontinuation Visit, if applicable. Scores for each cranial nerve were captured as outlined in Table 12 above. Examination procedures are as outlined in Table 31.

TABLE 31
Cranial Nerves II-VII Evaluation Criteria

| Cranial | | | |
|---|---|---|---|
| Nerve | Nerve Name | Function | Test |
| II | Optic Nerve | Pupillary reaction | Tell the subject to look into the distance. Shine a bright |
| III | Oculomotor | to light and | light obliquely into each pupil. Look for: |
| | Nerve | accommodation | Direct light reflex-Pupillary constriction in the same eye. |
| | | | Consensual reaction-Pupillary constriction |
| III | Oculomotor | Extraocular | Stand two feet directly in front of subject. Use your finger |
| | Nerve | movements | to make a horizontal sweep from the subject's left to the |
| IV | Trochlear Nerve | | right at the level of the subject's eyes. Repeat this |
| VI | Abducens | | horizontal sweep at the level of the forehead and the chin. |
| | | | Look for normal conjugate movement of the eyes in each |
| V | Trigeminal | Motor | Ask the subject to clench their teeth. Palpate the temporal |
| | Nerve | | and masseter muscles and note |
| | | Gross sensation | Tell subject to close their eyes. Using a cotton swab stick |
| | | | or similar object, touch the subject's forehead, cheeks |
| | | | and jaw with the dull cotton end and the sharper wooden |
| | | | end in. |
| VII | Facial Nerve | Motor | The Regional House-Brackmann Facial Nerve Grading |
| | | | System will be used to evaluate the facial nerve branches |
| | | | at rest and during conversation with the subject. Ask the |
| | | | subject to |
| | | | Raise both eyebrows |
| | | | Frown |
| | | | Close both eyes tightly while Investigator tries to open |
| | | | eye lids |
| | | | Show both upper and lower teeth |

Regional House-Brackmann Facial Nerve Grading System: This system was designed to evaluate synkinesis and the 4 major branches of the facial nerve (VII) that innervates target and adjacent musculature (Yen, et al., 2003, Oto!Neurotol. 24(1):118-122). The Investigator evaluated functionality of the facial nerve (VII) at Screening, Treatment Visit pre- and post-treatment, Follow-up Visits, and Final Evaluation Visit or Early Discontinuation Visit, if applicable. Refer to Table 13, above, and Table 32 below.

TABLE 32

| | | |
|---|---|---|
| Forehead | 1 | Normal forehead movement |
| | 2 | Slight weakness in forehead movement |
| | 3 | Obvious but not disfiguring asymmetry with motion, symmetric at rest |
| | 4 | Obvious weakness of disfiguring asymmetry with motion, symmetric at rest |
| | 5 | Barely perceptible motion in forehead, asymmetric at rest |
| | 6 | No movement |
| Eye | 1 | Normal eye closure |
| | 2 | Mild weakness in eye closure |
| | 3 | Obvious weakness but able to close eyes |
| | 4 | Unable to close eye with maximal effort |
| | 5 | Barely perceptible eyelid movement |
| | 6 | No movement |
| Midface | 1 | Normal midface movement |
| | 2 | Slight weakness in midface movement |
| | 3 | Obvious but not disfiguring weakness, symmetric at rest |
| | 4 | Obvious weakness and disfiguring asymmetry with motion, symmetric at rest |
| | 5 | Barely perceptible motion in midface, asymmetric at rest |
| | 6 | No movement |
| Mouth | 1 | Normal corner of mouth movement |
| | 2 | Slight weakness of corner mouth movement |
| | 3 | Obvious but not disfiguring weakness, symmetric at rest |
| | 4 | Obvious weakness and disfiguring asymmetry with motion, symmetric at rest |
| | 5 | Barely perceptible corner of mouth movement, asymmetric at rest |
| | 6 | No movement |
| Synkinesis | 1 | None |
| | 2 | Mild - obvious but not disfiguring |
| | 3 | Severe - disfiguring or interferes with function |

Evaluation of Facial Muscle Strength: Facial muscle strength was evaluated using the Medical Research Council (MRC) Scale for Assessment of Muscle Power (Patemostro-Sluga, et al., 2008, J Rehabil Med40:665-671). The following muscles on each side of the face were evaluated: orbicularis oculi (eyelid), lateral brow elevators, zygomaticu. See Table 14. The Investigator evaluated facial muscle strength at Treatment Visit pre- and post-treatment, Follow-up Visits and Final Evaluation Visit or Early Discontinuation Visit, if applicable.

Adverse Events: Adverse Events (AEs) were graded as mild, moderate, or severe, and evaluated at the Treatment Visit post-treatment, Follow-up Visits, and Final Evaluation Visit or Early Discontinuation Visit, if applicable.

Distant Spread of Toxin Query (Specific AE and Symptoms of Neuromuscular Weakness Query): Distant Spread of Toxin Query was conducted at the Treatment Visit pre- and post-treatment, Follow-up Visits, and Final Evaluation Visit or Early Discontinuation Visit, if applicable. Patients were queried in a general manner on the list of adverse events potentially suggestive of distant spread of toxin, including accommodation disorder, are flexia, aspiration, blurred vision, botulism, eyelid function disorder, eyelid ptosis, facial palsy, facial paresis, fourth cranial nerve paresis, peripheral nerve palsy, peripheral paralysis, pelvic floor muscle weakness, pneumonia aspiration, pupillary reflex impaired, bradycardia, brow ptosis, bulbar palsy, constipation, cranial nerve palsies, cranial nerve paralysis, diaphragmatic paralysis, diplopia, dry mouth, dysarthria, dysphagia, dysphonia, dyspnea, extraocular muscle paresis, paresis, gastrointestinal disorders, quadriparesis, headaches, hemiparesis, hypoglossal nerve paresis, hyporeflexia, hypotonia, monoparesis, muscular weakness, paralysis, paralysis flaccid, paralytic ileus, paraparesis, paresis cranial nerve, respiratory failure, respiratory arrest, respiratory depression, speech disorder, third cranial nerve paresis, trigeminal nerve paresis, urinary retention, vocal cord paralysis, vocal cord paresis, and xerophthalmia (dry eyes).

Effectiveness Evaluations: Investigator Global Assessment Frown Wrinkle Severity (IGA-FWS); Patient Frown Wrinkle Severity (PFWS); and Investigator and Patient Global Aesthetic Improvement Scale (GAIS). Assessments are as described above.

Other Evaluations: photographs of treatment area. Standardized digital photographs of the treatment area were taken. For photos to assess for presence of ptosis, either at baseline for comparison purposes, or at subsequent visits where reported or observed signs of suspected ptosis are present, photographs were taken in a standardized manner with the sponsor-supplied camera. The two photos taken at each time point were 1) of the subject in primary gaze with brow relaxed, and 2) of the subject in primary gaze with the brow elevated. Photographs also included subject's forehead at maximum forehead raise and at rest afterward maximum forehead raise.

Diagnosis and Main Abbreviated Eligibility Criteria: Outpatient, male or non-pregnant, non-nursing females, 18 years of age or older, and in good general health with moderate (2) or severe (3) glabellar lines at maximum frown based on the IGA-FWS and PFWS.

Test Article, Dose, Administration: RT002, 40 U, IM injection, 0.5 mL. The RT002 product is an injectable formulation, which contains the 150 kD subtype A botulinum toxin molecule without accessory proteins, which is non-covalently associated with a positively charged carrier peptide having the formula RKKRRQRRRG-(K)15-GRKKRRQRRR (RTP004; SEQ ID NO: 4), and which does not contain accessory proteins or animal-derived components. The excipient comprises 0.1 mg polysorbate 20, 36 mg trehalose dihydrate, and 11.7 μg RTP004, per 50 U of the 150 kDa type A toxin without accessory proteins and the treatment dose is 40 U.

Statistical Analyses: All statistical programming will be performed using statistical analysis system (SAS) version 9.4 or higher.

The trial subjects were eligible to receive up to three treatments. For analysis purposes, the corresponding summary period was defined for each treatment. For the trial-overall summary, all available data observed during the trial was included. For analyses associated with a specific treatment, the summary included all data observed since the treatment until the next treatment, or until the last visit of the trial when there is no subsequent treatment. To account for varying subject follow-up duration, the total follow-up duration (i.e., patient-years) was calculated for each summary period. For the trial-overall summary, the baseline was the last available value prior to the first treatment. For summaries associated with a specific treatment, the baseline was the last available value prior to treatment (i.e., re-baselined).

The Safety-Evaluable population included all subjects who are exposed to the investigational product and who provide any post-treatment safety information. Analyses specifically associated with each of the three treatment periods were performed on a subset of the safety-evaluable population, including only those subjects who received trial treatment and have post-treatment safety information for the specific treatment. These safety-evaluable sub-populations were respectively identified as Treatment-I-Evaluable, Treatment-2-Evaluable, or Treatment-3-Evaluable.

For the roll over subjects who were in the active treatment group in the prior trial, the first RT002 treatment in this open-label safety trial was in fact their second RT002 treatment. Based on the subject's prior exposure to RT002, the following two summary groups were defined for the analysis: Group A and Group B. The former group included all subjects who have received RT002 in either arm I or arm 2 of Example 7, and the latter group will include the remaining trial subjects.

Safety Analyses: All treatment-emergent adverse events (AEs) occurring during the trial were recorded and classified on the basis of MedDRA terminology, and as described herein. An AE can be defined as any untoward medical occurrence (e.g., sign, symptom, disease, syndrome, intercurrent illness, clinically significant abnormal laboratory finding, injury, or accident) that emerges or worsens following administration of investigational product and until the end of trial participation that may not necessarily have a causal relationship to the administration of the investigational product. An AE can therefore be any unfavorable and/or unintended sign (including a clinically significant abnormal laboratory result), symptom, or disease temporally associated with the use of an investigational product, whether or not considered related to the investigational product. A treatment-emergent AE is one that occurs after any period of exposure to treatment. AE's include any clinically significant change in the trial safety evaluations (e.g., vital signs, injection site evaluation, assessment of cranial nerves II-VII, and evaluation of facial muscle strength) post-treatment.

Pre-existing conditions, which increase in frequency or severity, or a change in nature as a consequence of an investigational product use, will also be considered an adverse event. An unexpected AE is an adverse reaction, the nature or severity of which is not consistent with the applicable product information.

A serious adverse event (SAE) is any untoward medical occurrence that results in any of the following outcomes: death, life-threatening, persistent or significant disability/incapacity or substantial disruption of the subject's ability to carry out normal life functions, requires in-patient hospitalization or prolongs hospitalization (i.e., a prolonged hospitalization beyond the expected length of stay; congenital anomaly/birth defect (i.e., an adverse outcome in a child or fetus of a subject exposed to the investigational product before conception or during pregnancy), does not meet any of the above serious criteria but based upon appropriate medical judgment may jeopardize the subject or may require medical or surgical intervention to prevent one of the outcomes listed above (i.e., is a significant or important medical event).

Safety data collected for the overall trial period were summarized for the Safety-Evaluable population. Summaries associated with each of the three treatment periods were performed on the corresponding sub-population (i.e., Treatment-I-Evaluable, Treatment-2-Evaluable or Treatment-3-Evaluable). To account for varying subject follow-up duration, the total follow-up duration (i.e., patient-years) was calculated for each summary period. Descriptive statistics were presented to summarize the safety data.

Investigational Product Causality And Severity: Relationship of an AE to investigational product was assessed as follows: Definite means that there is a clinically plausible time sequence between the onset of the AE and the administration of investigational product; when the event responds to withdrawal of investigational product and/or recurs with re-administration of investigational product; Probable means that there is a clinically plausible time sequence between the onset of the AE and the administration of investigational product; the AE is unlikely to be caused by the concurrent/underlying illness, other drugs or procedures; Possible means that there may or may not be a clinically plausible time sequence between the onset of the AE and the administration of investigational product and a cause cannot be ruled out; Unrelated: means that there is not a temporal or causal relationship to investigational product administration.

AE Category: Mild means that the event may be noticeable to subject; does not influence daily activities; usually does not require intervention; moderate means that the event may be of sufficient severity to make subject uncomfortable; performance of daily activities may be influenced; intervention may be needed; severe means that the event may cause severe discomfort; usually interferes with daily activities; subject may not be able to continue in the trial; treatment or other intervention usually needed.

All reported treatment-emergent adverse events were summarized, in terms of the number of subjects reporting events, system organ class, preferred term, severity, relationship to trial drug, and seriousness. When summarizing events by causality and severity, each subject was counted only once within a system organ class or a preferred term by using the event with the greatest relationship and highest severity within each classification. A list of AEs that lead to the subject's premature discontinuation of the trial were provided. Serious adverse events (SAEs) were listed by subject, summarized by severity, and relationship to trial treatment. The summaries of AEs and SAEs were performed for the trial overall, and for each of the three treatment periods. For SAEs and key AEs (such as those suggesting distant spread of toxin), rate-per-injection and rate-per-patient-year were calculated. A 95% confidence interval was also provided for the rate.

Effectiveness Analyses: Effectiveness outcome measures, such as the IGAFWS, PFWS, GAIS, were evaluated at maximum frown and at rest after maximum frown over time during the trial. Response rates and duration of the response were calculated. Effectiveness data were summarized as observed with no imputation for missing data. Descriptive statistics were provided for all effectiveness variables at all timepoints for the summary group. 95% confidence intervals and/or p-values for comparing the difference between subgroups of interest (e.g., females vs. males, first treatment vs. second treatment, etc.) were provided as appropriate. Kaplan-Meier curves were plotted for the time-to-event endpoints. When comparisons (e.g., females vs. males, first treatment vs. second treatment, etc.) were performed, the tests were done at a significant level of 0.05 with no adjustment for multiplicity.

Sample Size Justification: This was a safety trial with all subjects treated with the same investigational product. The sample size of approximately 2,100 was considered adequate in assessing safety based on several approaches. With approximately 2, 100 subjects being treated in this trial, it was concluded that with a 95% confidence, the incidence of an untoward event is no more than 0.15% when no such an event occurred during the trial. With n=2,100, the precision (based on a 95% Exact confidence interval) of estimating an event incidence was at most ±2.22% when the true incidence is around 50%, or approximately ±1.0% when the true incidence is in the range of 5%. The size of n=2,100 also had an adequate power to evaluate whether the incidence of a certain event is equivalent to a fixed incidence, for example, there is a power of >95% to demonstrate that the incidence is equivalent to 3% under an equivalent margin of 1.5% and a significant level of 0.05 (based on two one-sided tests).

With at least 400 of the 2,100 enrolled subjects receiving up to three treatments, there was a 86% power to detect an increase in incidence from the first treatment to the third treatment at a significant level of 0.05 (I-sided test) when the actual event incidence increases from 3% at the first treatment to 6% at the third treatment. Also, with n=400, the 95% Exact confidence interval was 1.6-5.2% for an observed incidence of 3%, and 3.1-7.6% for an observed incidence of 5%.

### (a) Trial Endpoints

Safety Endpoints: Incidence, severity, and relationship to trial drug of treatment-emergent adverse events during each treatment and the trial overall; and incidence, severity, and relationship to trial drug of treatment-emergent serious adverse events during each treatment and the trial overall. Safety evaluable population includes all subjects exposed to the investigational product.

Effectiveness Endpoints: Unless specified otherwise, all endpoints associated with IGA-FWS, PFWS or GAIS henceforth were based on assessments at maximum frown. When applicable, similar endpoints based on assessments at rest after maximum frown were also summarized. For the endpoints that are derived from a comparison with the baseline, two derivation rules using different reference timepoints as the baseline (i.e., trial baseline or treatment baseline) were applied separately. These include: time to retreatment since the first trial treatment (on Treatment-I-Evaluable only); time to retreatment since the second trial treatment (on Treatment-2-Evaluable only); time to return to, or worse than, baseline on both IGA-FWS and PFWS; time to return to 2 or 3 (moderate or severe) on both IGA-FWS and PFWS; proportion of subjects with a 2:2 point improvement from baseline on both IGAFWS and PFWS at each visit over time; proportion of subjects with a score of O or I (none or mild) on IGA-FWS at each visit over time; proportion of subjects with a score of O or I (none or mild) on PFWS at each visit over time; proportion of subjects with a 2:1 point improvement from baseline on both IGAFWS and PFWS at each visit over time; proportion of subjects with a 2:1 point improvement (i.e., improved, much improved, or very much improved) on GAIS at each visit over time (with investigator's assessment and subject's self-assessment summarized separately); proportion of subjects with a 2:2 point improvement (i.e., much improved, or very much improved) on GAIS at each visit over time (with investigator's assessment and subject's self-assessment summarized separately); proportion of subjects with a 2:3 point improvement (i.e., very much improved) on GAIS at each visit over time (with investigator's assessment and subject's self-assessment summarized separately); mean GAIS score at each visit over time (with investigator's assessment and subject's self-assessment summarized separately).

### (a) Key Effectiveness Endpoints:

Proportion of subjects with a 2:2 point improvement from baseline on both IGA-FWS and PFWS over time;
Proportion of subjects with a score of 0 or 1 (None or Mild) as evaluated by IGA-FWS and PFWS over time;
Time to loss of None or Mild on both IGA-FWS and PFWS;
Time to return to, or worse than, baseline on both IGA-FWS and PFWS; and

Proportion of subjects with a 2:1 point improvement on GAIS as evaluated by investigator and subject over time.

The key effectiveness endpoints were conducted for trial-overall summary and by treatment period.

Brief Comparison to Example 7: Both investigators and subjects trained at trial initiation on a validated 4-Point Wrinkle Severity Scale to assess severity of glabellar frown lines. At entry, subjects were required to have glabellar line wrinkle severity of either Moderate or Severe as assessed by both the investigator and subject.

### (a) Results for Example 8

This study confirmed the safety profile established in two Phase 3 pivotal studies (Example 7), with no new tolerability or safety concerns reported, and stable or decreasing rates of AEs following repeat dosing. A total of 3830 treatments with RT002 (40U) were administered to 2691 subjects in this study. There was no evidence of cumulative AEs over 3 treatment cycles, suggesting the safety pattern of RT002 remained stable after repeated dosing.

Safety Summary: RT002 was well-tolerated across 3,800 treatments in glabellar (frown) lines; adverse events were mild, localized and transient; rate of treatment related AEs decreased over successive treatments; eyelid Ptosis rate was less than 1% per treatment. Treatment-related Adverse Events were experienced in fewer treatments (14%) in Example 8's study across 66 clinical trial sites, compared with the Example 7's Arms I and 2 studies (18%) conducted at 30 sites.

Summary of AEs: 39% of subjects experienced an adverse event during the course of the study. Overall, the incidence of TEAEs did not increase over successive treatment cycles and the majority of events were mild in severity with most considered not to be related to treatment. The most common adverse events reported were headache (5.9% subjects), nasopharyngitis (4.3%), injection site pain (3.3%), injection site erythema (2.9%), injection site oedema (2.6%), erythema (2.4%) and upper respiratory tract infection (2.0%). Serious AEs occurred in 1.1% of subjects and none were related to treatment.

Treatment-related AEs: 18% of subjects reported adverse events that were considered related to RT002 or the administration procedure. Treatment-related AEs were generally mild in severity. The most frequently occurring treatment-related AEs were headache (4.6% of subjects), injection site pain (3.2%), injection site erythema (2.7%), injection site oedema (2.6%) and erythema (2.0%). Progressively lower percentages of subjects experienced treatment-related AEs following repeat treatment. In subjects who received three successive treatments: for Treatment 1, 13.5% of subjects experienced treatment-related AEs, for Treatment 2, 4.1% of subjects experienced treatment-related AES; and for Treatment 3, 3.8% of subjects experienced treatment-related AEs.

Eyelid Ptosis: A low rate of eyelid ptosis of 0.9% (35 events in 3830 treatments) was observed, and occurred in 1.3% of subjects at 66 sites. This compares favorably to a rate of 2.2% in 405 subjects treated in the studies of Example 7 at 30 sites.

Surprisingly, the rate of eyelid ptosis occurrences decreased with subsequent RT002 treatment cycles: for Treatment 1, 1.0% of subjects experienced treatment-related AEs; for Treatment 2, 0.8% of subjects experienced treatment-related AES; and for Treatment 3, 0.7% of subjects experienced treatment-related AEs. All but one eyelid ptosis was unilateral m presentation, and the majority were mild in severity and had a median duration of 45 days.

Effectiveness/Efficacy Summary: This study demonstrated efficacy that exceeded or was comparable to that seen in the two arms of the study of Example 7 with RT002 at a dose of 40U. Efficacy of RT002 was highly consistent across multiple treatment cycles and multiple endpoints evaluating both response rates and duration of effect.

Efficacy results across Example 7-Example 8 represent the highest responder rates and longest duration of effect observed in registration trials for moderate to severe glabellar lines. Effectiveness of DAXI (RT002 for these clinical trials) was maintained beyond a single treatment with a very high proportion of subjects meeting the treatment goal of None or Mild wrinkle as early as Week 1. Overall, there was a greater than 90% response at Week 1; efficacy exceeded or was comparable to that seen in Example 7.

None or Mild response rates increased through Week 4 across all 3 treatment cycles to at least 95% of investigators on IGA-FWS and 91% of subjects on PFWS, confirming the robust response observed in Example 7 Arms 1 and 2. At week 4, subjects in Example 8 achieved a greater than 95% response rate, and a high 2-point composite response rate, which increased with each successive treatment cycle: Treatment cycle 1=73.2%, treatment cycle 2=77.5% and treatment cycle 3=79.6%. Example 8 results confirm the high rate of 2-point composite response observed at week 4 in subjects participating in the placebo-controlled Example 7 Arms 1 and 2 studies of 73.6% and 74.0%, respectively.

Responder Rates Summary: The results of this study represent the highest responder rates observed in registration trials for glabellar lines. A very high proportion of subjects met goals of treatment. At week 4, 95% of investigators, and 91% of subjects rated the subject as having none or mild glabellar lines (on the IGA-FWS PFWS scales, respectively) across all treatment cycles. Further, 99% of investigators, and 97% of subjects reported improvement in glabellar lines at week 4 in all cycles of treatment on the GAIS. More than 70% of investigators and more than 60% of subjects reported maintaining improvement at week 24 in cycles 1 and 2. Also, the proportion of subjects meeting the FDA-mandated 2-point composite response endpoint was comparable with arms 1 and 2 of Example 7, with responder rates of 73.2%, 77.5% and 79.6% at week 4 in treatment cycles 1, 2 and 3 respectively.

Repeat dosing of RT002 was effective, with an extended duration of effect and high response rates that increased over time, in subjects receiving 3 treatments (n=340):
2 point improvement IGA: 73.2%/77.7%/79.6%
None or Mild IGA scores: 95.8%/96.6%/97.7%

Duration of Effect Summary: Example 8 results confirm the long duration of clinical benefit observed with the 40U dose of RT001 in Example 7's Aims 1 and 2 and demonstrate consistency across treatment cycles. Median time to return to baseline glabellar line severity was 28 weeks in treatment cycles 1 and 2 and is also consistent with the time taken to loss of complete treatment effect observed in the pivotal studies of Example 7's Arms 1 and 2. Median time to loss of none or mild wrinkle severity was 24 weeks in Example 8 treatment cycles 1 and 2, as evaluated by both investigator and subject and identical to duration of clinical benefit observed in Example 7's Arms 1 and 2.

Duration of effect in Age and Prior Toxin Exposure subgroups was similar or greater than overall Example 8 population, demonstrating predictability and consistency of RT002 in treatment of glabellar lines. By Age: time to return to baseline wrinkle severity in subjects aged 65 or older was 31 weeks, compared with 28 weeks for subjects less than 65 years and for the overall Example 8 population. Subjects aged 65 or greater experienced the same time to loss of none or mild wrinkle severity with a median duration of 24 weeks as those subjects less than 65 years and the overall Example 8 population. By Prior Toxin Exposure: Median time to return to baseline wrinkle severity was 28 weeks and median time to loss of none or mild wrinkle severity was 24 weeks regardless of prior BoNTA exposure at the time of RT002 treatment.

Consistency summary: Safety, efficacy, and duration exceeded or was comparable to that seen in Example 7; safety profile consistent with Example 7 Arms 1 & 2 and other approved neuromodulators; extended duration of effect was seen across all treatment groups; including age and toxin treatment experience.

Conclusions: This study evaluated the long term efficacy and safety of multiple treatments of RT002 for treatment of moderate to severe glabellar lines (GL). The interval between each treatment was at least 12 weeks with a maximum treatment duration of 36 weeks. The study provides up to 84 weeks of follow-up data and more extensive exposure to study drug than Example 7's trials. This study is the first long term study of RT002 to mimic repeated administration over time in a clinical setting. A total of 2691 subjects were enrolled, with 1033 subjects receiving multiple (up to 3) treatments.

High response rates and long duration of effect was observed following single treatment and sustained over multiple treatments in this study, reaffirming the outcomes seen in Example 7's single treatment studies in a larger study, with a broader selection of clinical sites.

Robust efficacy observed in single treatment Example 7 studies was maintained with successive RT002 treatments and no new safety issues were observed, while the incidence of treatment-related AEs declined with successive treatments. This study demonstrated that up to 3 treatments of glabellar lines with RT002 over 84 weeks were well-tolerated, and that over 95% patients reached treatment goal of None or Mild severity, with duration of effect, defined as median time to loss of None or Mild response, lasting 24 weeks.

Additional details of the study Results are as follows.

This study enrolled nearly 2,700 subjects, meeting a benchmark of at least 2,100 single treatments and 500 triple treatments.

Subject Disposition: see Table 33 and Tables 34A-34B.

TABLE 33

| Disposition | N (%) |
|---|---|
| Number of Subjects Enrolled | 2,691 |
| Number of Subjects who Completed Study | 2,314 (86.0%) |
| Number of Subjects who Discontinued Study | 377 (14.0%) |
| Reasons for Discontinuation | |
| Withdrawal by Subject | 196 (7.3%) |
| Lost to Follow up | 99 (3.7%) |
| Protocol Deviation | 53 (2.0%) |
| Other | 15 (0.6%) |
| Investigator Discretion | 8 (0.3%) |
| Adverse Event | 5 (0.2%) |
| Death* | 1 (0.40%) |

*Secondary to homicide

TABLE 34A

| | Total in Example |
|---|---|
| Number of Subjects | 2,691 |
| RT002 40U Treatment Received in Ex. 8 | |
| Treatment Cycle #1 | 2,380 |
| Treatment Cycle #2 | 882 |
| Treatment Cycle #3 | 568 |
| Total Number of Treatments | 3,830 |

TABLE 34B

| | RT002 40 U Treatment | | | |
|---|---|---|---|---|
| | Eligibility in Example 8 | | | |
| | One* | Two | Three* | Total |
| Number of Subjects | 1658 | 477 | 556 | |
| RT002 40U Treatment Received in Ex. 8 | | | | |
| Treatment Cycle #1 | 1658 | 477 | 556 | |
| Treatment Cycle #2 | — | 362 | 437t | |
| Treatment Cycle #3 | — | — | 340 | |
| Total Number of Treatments | 1658 | 839 | 1333 | 3830 |

*De novo subjects; Subjects from Example 7, Arms 1 and 2; t51 subjects were eligible to receive Treatment 3 due to maintenance of treatment effect at Week 36.

Demographics compared to Example 7: The treatment population was similar to those in Arms 1 and 2 of Example 7. see Table 35 and Table 36

TABLE 35

| | Ex. 7 Arms 1 & 2 Pooled | | All Subjects | |
|---|---|---|---|---|
| | (N = 405) | | (N = 2691) | |
| Female, n (%) | 357 | (88.1%) | 2383 | (88.6%) |
| Age (years), | 50.2 | (1.46) | 49.5 | (11.27) |
| mean (SD) | [21, 74] | | [21, 86] | |
| range [min, max] | | | | |
| Race | | | | |
| White | 353 | (87.2%) | 2407 | (89.4%) |
| Black/African American | 19 | (4.7%) | 129 | (4.8%) |
| Asian | 18 | (4.4%) | 73 | (2.7%) |
| Other | 15 | (3.7%) | 83 | (3.0%) |
| Ethnicity | | | | |
| Hispanic/Latino | 66 | (16.2%) | 516 | (19.2%) |
| Not Hispanic/Latino | 339 | (83.5%) | 2175 | (80.8%) |

TABLE 36

| | Ex. 7 Arms 1 & 2 | | | |
|---|---|---|---|---|
| | Pooled | | All Subjects | |
| | (N = 405) | | (N = 2691) | |
| Prior BoNT-A*, n (%) | 213 | (52.6) | 1074 | (39.9%) |
| Months since last BoNT-A* | 27.4 | (1.59) | 34.9 | (41.78) |
| mean (SD) | [7, 205] | | [0.2, 320] | |
| range | | | | |
| PFWS at Maximum Frown | | | | |
| Moderate | 226 | (55.8) | 1482 | (55.1%) |
| Severe | 179 | (44.2%) | 1208 | (44.9%) |
| IGA-FWS at Maximum Frown | | | | |
| Moderate | 252 | (62.2%) | 1651 | (61.4%) |
| Severe | 153 | (37.8%) | 1040 | (38.6%) |

Safety Outcomes: Summary of Adverse Events compared with Example 7: The majority of AEs were mild in severity with no treatment related SAEs occurring in any trials of Example 7 and Example 8. See Table 37.

TABLE 37

| | Ex. 7 Arms 1 & 2 | | Ex. 8 | |
|---|---|---|---|---|
| | Pooled (N = 406) | | (N = 2691) | |
| | n (%) | | n (%) | |
| Death* | 0 | | 1 | (<0.1%) |
| Serious AEs (SAE) | 4 | (1.0%) | 29 | (1.1%) |
| Subjects with any AE | 166 | (40.9) | 1043 | (38.8%) |
| Mild | 124 | (30.5%) | 739 | (27.5%) |
| Moderate | 36 | (8.9%) | 267 | (9.9%) |
| Severe | 6 | (1.5%) | 37 | (1.4%) |
| Subjects with any AE leading to | 0 | | 5 | (0.2%) |
| Study Discontinuation | | | | |
| Treatment-Related Adverse Events** | | | | |
| Subjects with any Treatment-related | 78 | (19.2%) | 480 | (17.8%) |
| AE | | | | |
| Treatment-related SAE | 0 | | 0 | |

*Secondary to homicide;
**Considered by investigator to be possibly, probably, or definitely related to the treatment.

Table 38 shows adverse events regardless of causality (2:2%) compared with Example 7.

TABLE 38

| | Ex. 7 Arms 1 & 2 | | Ex. 8 | |
|---|---|---|---|---|
| | Pooled (N = 406) | | (N = 2691) | |
| Preferred Term | n (%) | | n (%) | |
| Headache | 37 | (9.1%) | 158 | (5.9%) |
| Nasopharyngitis | 17 | (4.1%) | 119 | (4.4%) |
| Injection site pain | 15 | (3.6%) | 105 | (3.9%) |
| Injection site erythema | 5 | (1.2%) | 90 | (3.3%) |
| Injection site oedema | 5 | (1.2%) | 76 | (2.8%) |
| Erythema | 1 | (0.2%) | 56 | (2.1%) |
| Upper respiratory tract | 11 | (2.7%) | 55 | (2.0%) |
| infection | | | | |

Table 39A-39B and Table 40 show treatment-related adverse events (2:1%) compared with Example 7. As shown in Table 39A, treatment-related AE rate was low and decreased with successive treatments in the study of Example 8.

TABLE 39A

| | Ex. 7 | | | | | |
|---|---|---|---|---|---|---|
| | Arms | | | | | |
| | 1 & 2 | | RT002 40U Treatments in Ex. 8 | | | |
| | Pooled | Ex. 8 | One | Two | Three | Total |
| Preferred | (N = 406) | (N = 2691) | (n = 2380) | (n = 882) | (n = 568) | (n = 3830) |
| Term | n (%) | n (%) | n (%) | n (%) | n (%) | n (%) |
| Any | 78 | 480 | 400 | 86 | 40 | 526 |
| Treatment- | (19.2%) | (17.8%) | (16.8%) | (9.8%) | (7.0%) | (13.7%) |
| Related AE | | | | | | |
| Headache | 26 | 124 | 102 | 13 | 12 | 127 |
| | (6.4%) | (4.6%) | (4.3%) | (1.5%) | (2.1%) | (3.3%) |
| Injection site | 15 | 98 | 81 | 18 | 7 | 106 |
| pain | (3.7%) | (3.6%) | (3.4%) | (2.0%) | (1.2%) | (2.7%) |
| Injection site | 5 | 81 | 64 | 22 | 8 | 94 |
| erythema | (1.2%) | (3.0%) | (2.7%) | (2.5%) | (1.4%) | (2.5%) |
| Injection site | 6 | 76 | 56 | 21 | 9 | 86 |
| oedema | (1.5%) | (2.8%) | (2.7%) | (2.4%) | (1.6%) | (2.2%) |
| Eyelid ptosis* | 9 | 33 | 23 | 7 | 4 | 34 |
| | (2.2%) | (1.2%) | (1.0%) | (0.8%) | (0.7%) | (0.9%) |

*29 (85%) cases mild severity, 5 (15%) cases moderate seventy.
Median resolution time 45 dys.

TABLE 39B

| | Ex. 7 Arms 1 & 2 | | | | Ex. 8 | | | |
|---|---|---|---|---|---|---|---|---|
| Preferred | Pooled (N = 406) | | | | (N = 2691) | | | |
| Term | n (%) | | | | n (%) | | | |
| Headache | 26 | | (6.4%) | | 124 | | (4.6%) | |
| Injection site | 15 | | (3.7%) | | 98 | | (3.6%) | |
| pain | | | | | | | | |
| Injection | 5 | | (1.2%) | | 81 | | (3.0%) | |
| site erythema | | | | | | | | |
| Injection | 6 | | (1.5%) | | 76 | | (2.8%) | |
| site oedema | | | | | | | | |
| Eyelid ptosis* | 9 | | (2.2%) | | 34 | | (1.3%) | |
| | RT002 40 U Treatments | | | | | | | |
| | Cycle One | | Cycle Two | | Cycle Three | | Total | |
| Overall Study | (n = 2380) | | (n = 882) | | (n = 568) | | (n = 3830) | |
| Eyelid ptosis* | 23 | 1% | 7 | (0.8% | 4 | (0.7% | 34 | (0.9% |

*29 (85%) cases mild severity, 5 (15%) cases moderate severity. Median resolution time 45 dys.

TABLE 40

| | Ex. 7 | | | | | |
|---|---|---|---|---|---|---|
| | Arms | | | | | |
| | 1 & 2 | Ex. 8 | | | | |
| | Pooled | Overall | | | | |
| Preferred | (N = 406) | (N = 2691) | One | Two | Three | Total |
| Term | n (%) | n (%) | (n = 2380) | (n = 882) | (n = 568) | (n = 3830) |
| Headache | 26 | 124 | 102 | 13 | 12 | 127 |
| | (6.4%) | (4.6%) | (4.3%) | (1.5%) | (2.1%) | (3.3%) |
| Injection site | 15 | 98 | 81 | 18 | 7 | 106 |
| pain | (3.7%) | (3.6%) | (3.4%) | (2.0%) | (1.2%) | (2.7%) |
| Injection site | 5 | 81 | 64 | 22 | 8 | 94 |
| erythema | (1.2%) | (3.0%) | (2.7%) | (2.5%) | (1.4%) | (2.5%) |
| Injection site | 6 | 76 | 56 | 21 | 9 | 86 |
| oedema | (1.5%) | (2.8%) | (2.7%) | (2.4%) | (1.6%) | (2.2%) |
| Eyelid ptosis* | 9 | 33 | 22 | 7 | 4 | 33 |
| | (2.2%) | (1.2%) | (0.9%) | (0.8%) | (0.7%) | (0.8%) |

Table 41 shows AEs across treatment cycles among subjects receiving all three treatments (n=340).

TABLE 41

| | RT002 40 U Treatment Cycle | | | | | |
|---|---|---|---|---|---|---|
| Overall Study | One | | Two | | Three | |
| Any Adverse Event | 116 | (34.1%) | 89 | (34%) | 53 | (15.6%) |
| Adverse Event by | | | | | | |
| Severity | | | | | | |
| Mild | 85 | (25.0%) | 56 | (16.5%) | 36 | (10.6%) |
| Moderate | 28 | (8.2%) | 31 | (9.1%) | 17 | (5.0%) |
| Severe | 3 | (0.9%) | 2 | (0.6%) | 0 | |
| Treatment Related AE* | 13.5% | | 4.1% | | 3.8% | |
| Preferred Term (>2%) | | | | | | |
| Headache | 16 | (4.7%) | 3 | (0.9%) | 2 | (0.6%) |
| Erythema | 8 | (2.4%) | 2 | (0.6%) | 0 | |
| Oedema | 7 | (2.1%) | 2 | (0.6%) | 0 | |

*Considered by investigator to be possibly, probably, or definitely related to the treatment.

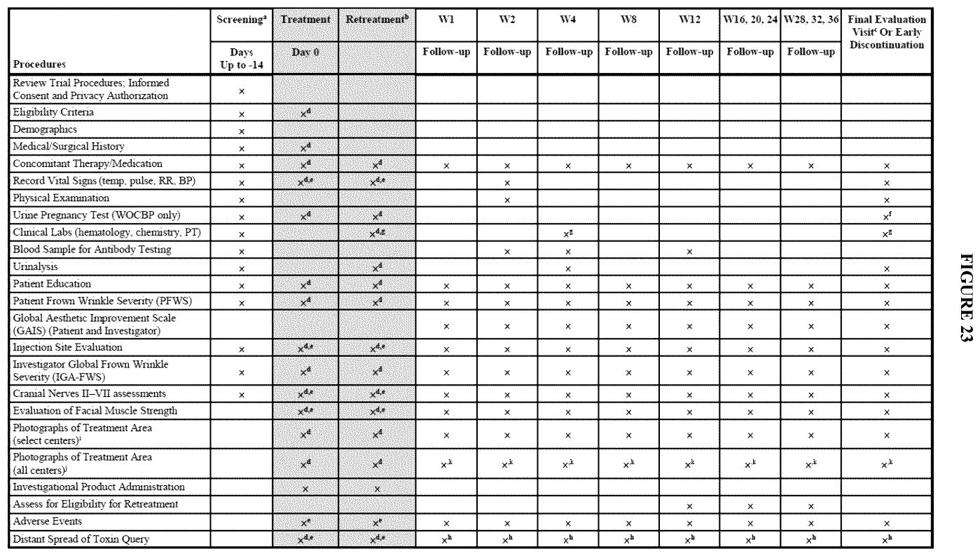

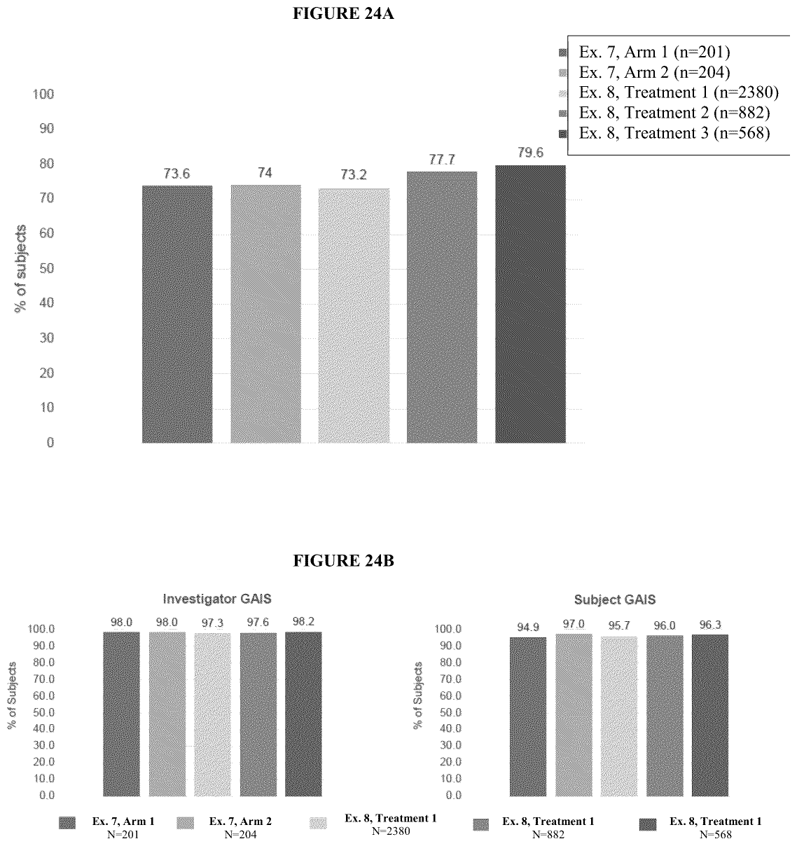

Efficacy Outcomes: FIGS. 24A-24B depict proportion of subjects maintaining improvement in glabellar lines at Week 4 across studies. FIG. 24A depicts proportion of subjects who achieve 2:2 point composite response at maximum frown at Week 4 in Example 7's Arm 1 & 2 and Example 8. As shown, the 2-point composite response was comparable across studies and treatments. Further, RT002 significantly improved the appearance of glabellar line severity, and the treatment response increased over successive treatment cycles. FIG. 24B depicts subjects who achieve at least a score of +1 on both Investigator and Subject GAIS scores at Week 4 across the Phase 3 studies of Example 7, Arms 1 & 2, and Example 8.

Table 42A and Table 42B show percent of subjects in Example 7 and Example 8 having wrinkle scores of “none” or “mild” in response to treatment at various time points following the treatment, as assessed by IGA-FWS and PFWS. As shown, there were robust response rates on key measures for none/mild outcome.

TABLE 42A
Investigator Assessment (IGA-FWS)

| | Ex. 7 | | | |
|---|---|---|---|---|
| | Arms | | | |
| | 1 & 2 | Ex. 8 Treatment | | |
| | Pooled (%) | ONE (%) | TWO (%) | THREE (%) |
| WEEK | n = 406 | n = 2380 | n = 882 | n = 568 |
| 1 | 93.8 | 91.8 | 91.5 | 92.3 |
| 2 | 95.8 | 96.6 | 93.9 | 96.1 |
| 4 | 97.5 | 95.8 | 96.6 | 97.7 |
| 8 | 93.1 | 91.6 | 92.2 | 93.7 |
| 12 | 86.2 | 81.2 | 83.9 | 84.5 |
| 16 | 72.6 | 67.6 | 59.4 | — |
| 20 | 53.8 | 50.3 | 45.2 | — |
| 24 | 32.3 | 34.0 | 32.7 | — |
| 28 | 15.1 | 22.6 | 20.9 | — |
| 32 | 7.7 | 15.0 | 10.4 | — |
| 36 | 4.0 | 7.3 | 4.8 | — |

TABLE 42B
Patient Assessment (PFWS)

| | Ex. 7 | | | |
|---|---|---|---|---|
| | Arms | | | |
| | 1 & 2 | Ex. 8 Treatment | | |
| | Pooled (%) | ONE (%) | TWO (%) | THREE (%) |
| WEEK | n = 406 | n = 2380 | n = 882 | n = 568 |
| 1 | 86.4 | 85.1 | 87.2 | 88.4 |
| 2 | 91.9 | 92.0 | 90.0 | 92.1 |
| 4 | 91.1 | 91.6 | 92.3 | 93.1 |
| 8 | 84.9 | 82.4 | 83.0 | 85.0 |
| 12 | 72.1 | 68.4 | 69.7 | 74.3 |
| 16 | 55.3 | 52.5 | 44.8 | — |
| 20 | 40.2 | 36.6 | 34.8 | — |
| 24 | 22.7 | 26.5 | 22.9 | — |
| 28 | 11.1 | 18.5 | 16.0 | — |
| 32 | 5.7 | 12.7 | 8.5 | — |
| 36 | 1.7 | 7.2 | 3.4 | — |

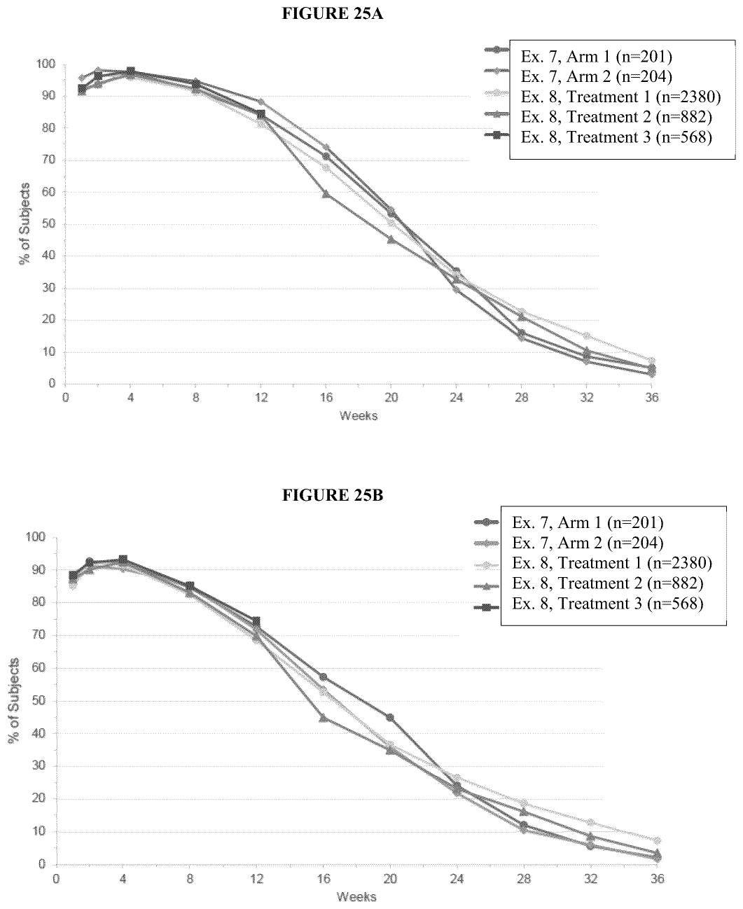

FIG. 25A and FIG. 25B depict this data graphically, showing percent of subjects in Example 7 and Example 8 having wrinkle scores of “none” or “mild” in response to treatment at various time points following the treatment, as assessed by IGA-FWS (FIG. 25A) and PFWS (FIG. 25B). As shown, outcomes were consistent in both cases between studies and cycles.

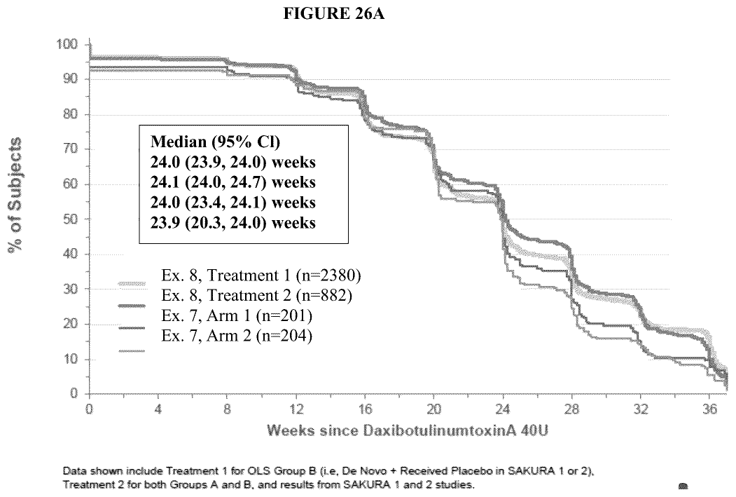

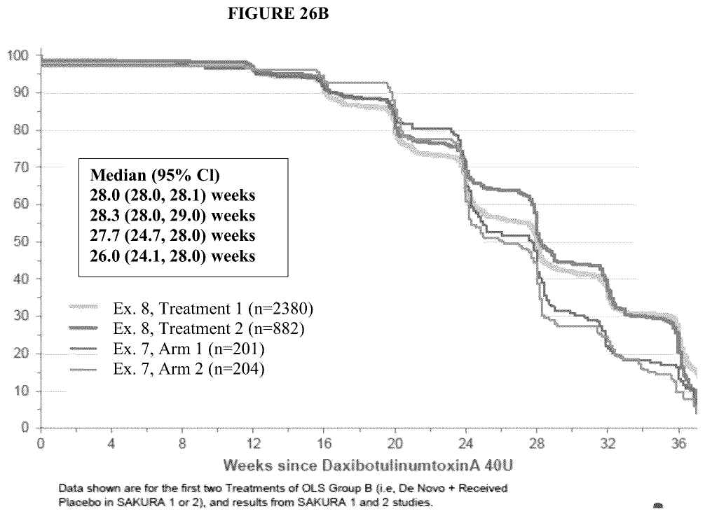

FIG. 26A and FIG. 26B depict percent of subjects in Example 7 and Example 8 versus time following treatment for loss of “none” or “mild” scores on both IGA-FWS and PFWS (FIG. 26A); and of loss to return to baseline on both IGA-FWS and PFWS (FIG. 26B). As shown in FIG. 26A, the median duration of 24 weeks was achieved for time to loss of none/mild wrinkle severity in treatment cycles one and two of Example 8 (that is, following first and second RT002 treatments), confirming time to loss of none/mild scores in Arms I & 2 of Example 7. As shown in FIG. 26B, the median duration of 28 weeks was achieved for time to return to baseline in treatment cycles one and two of Example 8 (that is, following first and second RT002 treatments), confirming time to return to baseline in Arms I & 2 of Example 7.

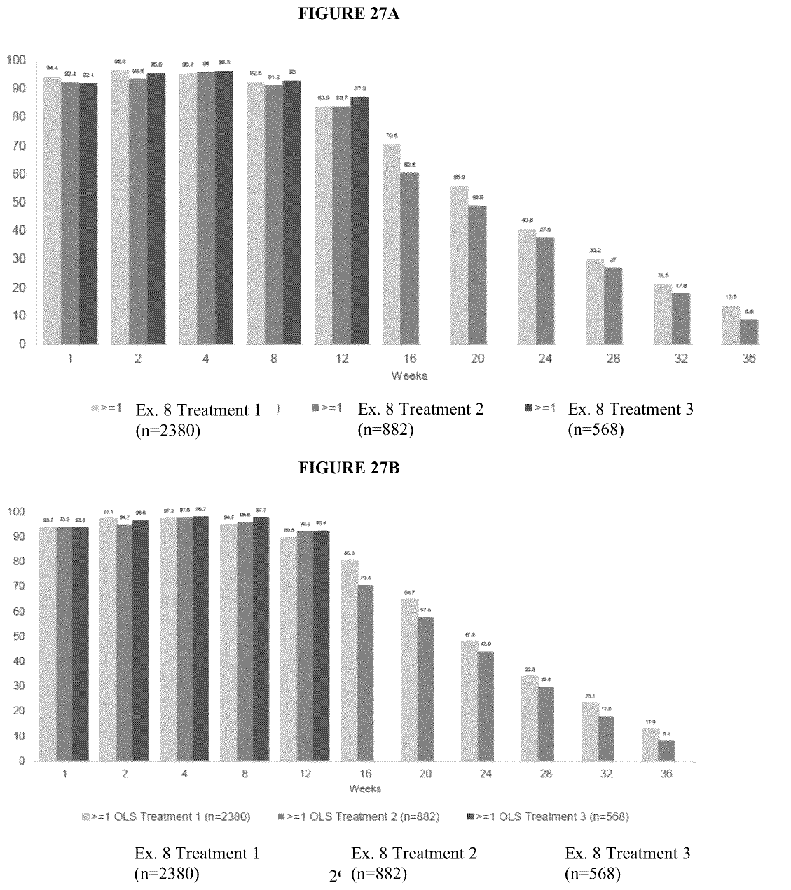

FIG. 27A and FIG. 27B depict percent of subjects in Example 8 showing a response as assessed by Subject's GAIS (P-GAIS) at maximum frown (FIG. 27A) or by Investigator's GAIS (1-GAIS) at maximum frown (FIG. 27B), over time following treatment. Response is a score greater than or equal to 1.

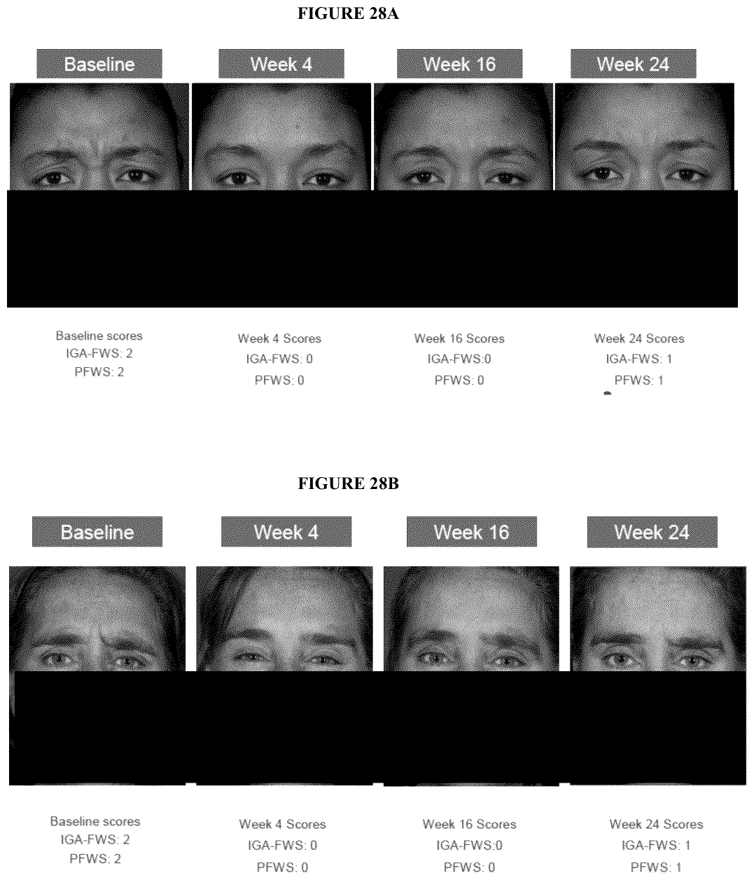

Photographs: FIG. 28A and FIG. 28B depict photographs of subjects exemplifying results discussed of this study. FIG. 28A depicts an example of 2-point improvement by IGA-FWS and PFWS at week 4, with sustained duration of effect through Week 16; and a I-point improvement remaining at Week 24. FIG. 28B depicts another example of 2-point improvement by IGA-FWS and PFWS at week 4, with sustained duration of effect through Week 16; and a I-point improvement remaining at Week 24.

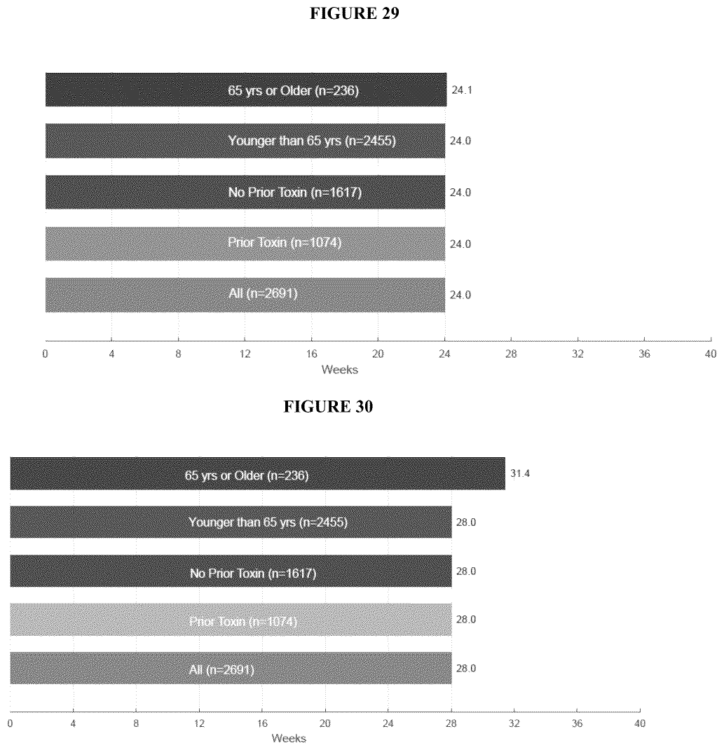

Subgroup Analysis: FIG. 29 and FIG. 30 provide additional information regarding response by subgroup.

FIG. 29 depicts median time to loss of none or mild scores on both IGA-FWS and PFWS by subgroup. As shown, treatment duration of 24 weeks was achieved regardless of age and prior toxin experience.

FIG. 30 depicts median time to return to baseline on both IGA-FWS and PFWS by subgroup. As shown, treatment duration of 28 weeks was achieved in subjects under 65 years regardless of prior toxin experience, with duration extended to 31.4 weeks in those 65 years of older.

Overall Summary: In this study, 2,691 patients were treated with RT002 with no new safety or tolerability concerns, provising a safety profile consistent with prior studies in Example 5 and Example 7. In all, 3,830 RT002 40 U injections were administered in the study of Example 8. In particular, a low rate of eyelid ptosis was observed, namely 0.9% (35 events in 3,830 treatments), and it occurred in 1.3% of subjects at 66 sites. This compares to rate of 2.2% in 405 subjects treated in Example 7's Arms 1 and 2 at 30 sites.

Example 8 demonstrated efficacy that exceeded or was comparable to that seen in Example 7's Arms 1 and 2 pivotal trials with RT002 at a dose of 40U. Example 8 results confirm the long duration of clinical benefit observed with the 40U dose of RT002 in Example 7's Arms 1 and 2, and demonstrate consistency across treatment cycles. The median time to Loss of None or Mild wrinkle severity was 24 weeks in both treatment cycle 1 and treatment cycle 2 in Example 8, as evaluated by both investigator and subject. This is identical to the duration of clinical benefit observed in the Example 7's Arms 1 and 2. The median time to return to baseline glabellar line severity was 28 weeks in treatment cycles 1 and 2 and is also consistent with the time taken to loss of complete treatment effect observed in the placebo-controlled Example 7's Arms 1 and 2 trials.

We claim:

1. A method of administering botulinum toxin to achieve an extended duration therapeutic or cosmetic effect in an individual, the method comprising:
administering by injection a treatment dose of a sterile injectable composition into an area of the individual in need thereof to achieve the therapeutic or cosmetic effect following treatment with the composition;
wherein the composition comprises a pharmaceutically acceptable diluent suitable for injection; and
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from O to 20;
wherein the treatment dose of the botulinum toxin component is administered to the individual is about 10 U to about 100 U per injection treatment;
wherein the positively charged carrier is non-covalently associated with the botulinum toxin component; and
wherein the treatment dose of the composition administered by injection to the individual achieves the extended duration therapeutic effect having at least about a 28-week duration of effect.

2. A method of reducing wrinkles, lines, or furrows in an individual in need thereof, the method comprising:
administering to the individual by injection to one or more muscles or facial structures associated with the glabellar lines of the individual a composition comprising:
a pharmaceutically acceptable diluent for injection;
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from 0 to 20;
wherein the botulinum toxin component is administered to the individual in a treatment dose amount of about 10 U to about 100 U per injection treatment.
wherein the positively charged carrier is non-covalently associated with the botulinum component; and
wherein the injection of the composition provides a single treatment dose having at least about a 28-week duration of effect in reducing wrinkles, lines, or furrows of the individual, thereby extending treatment interval duration for the individual.

3. A pharmaceutical composition in a sterile injectable formulation for use in administering botulinum toxin to achieve an extended duration therapeutic or cosmetic effect in an individual,
said composition comprising a pharmaceutically acceptable diluent suitable for injection;
a botulinum toxin component in a treatment dose of 10 U to 100 U, wherein said botulinum toxin component is selected from the group consisting of a botulinum toxin complex, a reduced botulinum toxin complex, or a botulinum toxin; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from 0 to 20;
wherein the positively charged earner is non-covalently associated with the botulinum toxin component; and
wherein said treatment dose of the composition achieves the extended duration therapeutic or cosmetic effect having at least about a 28-week duration of effect in the individual administered said formulation by injection.

4. A pharmaceutical composition in a sterile injectable formulation for use in reducing wrinkles, lines, or furrows in an individual in need thereof, said composition comprising:
a botulinum toxin component in a dose of about 10 U to about 100 U, said botulinum toxin component selected from the group consisting of a botulinum toxin complex, a reduced botulinum toxin complex, or a botulinum toxin,
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from O to 20; and
a pharmaceutically acceptable diluent for injection;
wherein the positively charged carrier is non-covalently associated with the botulinum toxin component; and
wherein said dose of the composition provides a single treatment having at least about a 28-week duration of effect in reducing wrinkles, lines, or furrows of the individual, thereby extending treatment interval duration for the individual.

5. The method according to claim 1 or claim 2, or the pharmaceutical composition for use according to claim 3 or claim 4, wherein the composition achieves an extended duration of effect for at least about 32 weeks.

6. The method or pharmaceutical composition for use according to claim 5, wherein the composition comprises botulinum toxin of serotype A

7. The method or pharmaceutical composition for use according to claim 6, wherein the composition comprises botulinum toxin of serotype A having a molecular weight of 150 kDa without accessory proteins.

8. The method or pharmaceutical composition for use according to any one of claims 1 to 7, wherein the positively charged polylysine backbone has covalently attached thereto one or more positively charged efficiency groups having the amino acid sequence (gly)p-RGRDDRRQRRR-(gly)q (SEQ ID NO: 1), wherein the subscripts p and q are each independently an integer of from Oto 20.

9. The method or pharmaceutical composition for use according to any one of claims 1 to 7, wherein the positively charged polylysine backbone has covalently attached thereto one or more positively charged efficiency groups having the amino acid sequence (gly)p-YGRKKRRQRRR-(gly)q (SEQ ID NO: 2), wherein the subscripts p and q are each independently an integer of from Oto 20.

10. The method or pharmaceutical composition for use according to any one of claims 1 to 7, wherein the positively charged polylysine backbone has covalently attached thereto one or more positively charged efficiency groups having the amino acid sequence (gly)p-RKKRRQRRR-(gly)q (SEQ ID NO: 3), wherein the subscripts p and q are each independently an integer of from Oto 20.

11. The method or pharmaceutical composition for use according to any one of claims 1 to 10, wherein (i) the subscripts p and q are each independently an integer of from Oto 8; or (ii) are each independently an integer of from 2 to 5.

12. The method or pharmaceutical composition for use according to any one of claims 1 to 11, wherein the one or more positively charged efficiency groups are attached to both ends of the positively charged polylysine backbone of the positively charged carrier.

13. The method or pharmaceutical composition for use according to claim 12, wherein the positively charged carrier has the amino acid sequence RKKRRQRRRG-(K)15-GRKKRRQRRR (SEQ ID NO: 4).

14. The method or pharmaceutical composition for use according to any one of claims 1 to 13, wherein the composition does not locally diffuse from the site of injection following injection.

15. The method or pharmaceutical composition for use according to any one of claims 1 to 14, wherein the treatment dose of botulinum toxin is administered to the individual in an amount of about 40 U per injection treatment.

16. The method or pharmaceutical composition for use according to any one of claims 1 to 15, wherein said positively charged carrier and said botulinum toxin component are present in said pharmaceutical composition in a mass ratio of about 20,000:1 to about 55,000:1.

17. The method or pharmaceutical composition for use according to claim 16, wherein said positively charged carrier is present in said pharmaceutical composition in an amount of about 8 μg to about 15 μg per 40 U of said botulinum toxin component.

18. The method or pharmaceutical composition for use according to any one of claims 1 to 17, wherein said excipient comprises at least one component selected from the group consisting of L-Histidine, L-Histidine hydrochloride, polysorbate 20, and trehalose dihydrate.

19. The method or pharmaceutical composition for use according to claim 18, wherein said excipient comprises trehalose dihydrate.

20. The method or pharmaceutical composition for use according to any one of claims 1 to 19, wherein said method or use comprises a single administration of said pharmaceutical composition.

21. The method or pharmaceutical composition for use according to any one of claims 1 to 20, wherein the therapeutic or cosmetic effect is reduction of a symptom associated with a disorder selected from the group consisting of hemifacial spasm, adult onset spasmodic torticollis, anal fissure, blepharospasm, cerebral palsy, cervical dystonia, plantar fasciitis, migraine headaches, strabismus, temporomandibular joint disorder, neurologic pain, overactive bladder, rhinitis, sinusitis, acne, dystonia, dystonic contractions, hyperhidrosis, and hypersecretion of a gland controlled by the cholinergic nervous system.

22. The method or pharmaceutical composition for use according to any one of claims 1 to 20, wherein the therapeutic or cosmetic effect is reduction of wrinkles, lines, or furrows of the individual.

23. The method or pharmaceutical composition for use according to claim 22, wherein the therapeutic or cosmetic effect is reduction in the severity of glabellar lines.

24. The method or pharmaceutical composition for use according to claim 22 or 23, wherein the administration comprises at least one injection into one or more muscle selected from the group consisting of the right corrugator muscle, the left corrugator muscle, and the procerus muscle.

25. The method or pharmaceutical composition for use according to claim 24, wherein about 8 U of said botulinum toxin component are injected into said the medial aspect of the right corrugator muscle, about 8 U of said botulinum toxin component are injected into said the lateral aspect of the right corrugator muscle; about 8 U of said botulinum toxin component are injected into said the medial aspect of the left corrugator muscle, about 8 U of said botulinum toxin component are injected into said the lateral aspect of the left corrugator muscle; and about 8 U of said botulinum toxin component are injected into a procerus muscle.

26. A sterile injectable composition comprising:
a botulinum toxin component selected from the group consisting of a botulinum toxin, a botulinum toxin complex, or a reduced botulinum toxin complex, in a dosage amount selected from about 10 U to about 100 U; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from O to 20; and
a pharmaceutically acceptable diluent for injection;
wherein the positively charged carrier is non-covalently associated with the botulinum toxin component;
wherein excipient comprises at least one component selected from L-Histidine, L-Histidine hydrochloride, polysorbate 20, and trehalose dihydrate;
wherein said positively charged carrier is present in said pharmaceutical composition in a mass ratio to said botulinum toxin component of about 20,000:1 to about 55,000:1; and
wherein the composition provides a therapeutic or cosmetic effect which endures for at least about 28 weeks following a single treatment of an individual with the injectable composition.

27. The composition according to claim 26, wherein the positively charged carrier has the amino acid sequence RKKRRQRRRG-(K)15-GRKKRRQRRR (SEQ ID NO: 4).

28. The composition according to claim 26 or claim 27, wherein the composition comprises botulinum toxin of serotype A having a molecular weight of 150 kDa without accessory proteins.

29. The composition according to any one of claims 26 to 28, wherein the treatment dose of the botulinum toxin component administered to the individual is about 40 U.

30. The composition according to any one of claims 26 to 29, wherein the excipient comprises trehalose dihydrate.

31. A method of treating an individual in need thereof with injectable botulinum toxin, wherein the method of treatment comprises a treatment course having multiple treatment intervals with prolonged duration of effect and duration of treatment intervals, the treatment course comprising:
administering by injection an initial treatment dose of a sterile injectable composition into an area of the individual in need thereof to achieve a therapeutic or cosmetic effect following the initial treatment with the composition;
wherein the composition comprises a pharmaceutically acceptable diluent suitable for injection;
a botulinum toxin component selected from the group consisting of a botulinum toxin, a botulinum toxin complex, or a reduced botulinum toxin complex; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RK.KRRQRRR-(gly)q (SEQ ID NO: 3), wherein the subscripts p and q are each independently an integer of from O to 20;
wherein the botulinum toxin component is administered to the individual in a treatment dose of about 10 U to about 100 U per injection treatment;
wherein the positively charged earner is non-covalently associated with the botulinum toxin component;
wherein the initial treatment dose of the composition administered by injection to the individual provides a therapeutic duration of effect lasting through at least about 28 weeks; and
administering subsequent treatment doses of the composition by injection to the individual at treatment intervals comprising a duration of greater than or equal to about 28 weeks to at least about 52 weeks following the initial treatment dose and between each subsequent treatment dose.

32. The method according to claim 31, wherein the composition comprises botulinum toxin of serotype having a molecular weight of 150 kDa without accessory proteins.

33. The method according to claim 31 or 32, wherein the positively charged polylysine backbone has covalently attached thereto one or more positively charged efficiency groups having the amino acid sequence (gly)p-RGRDDRRQRRR-(gly)q (SEQ ID NO: 1),
wherein the subscripts p and q are each independently an integer of from Oto 20.

34. The method according to claim 31 or 32, wherein the positively charged polylysine backbone has covalently attached thereto one or more positively charged efficiency groups having the amino acid sequence (gly)p-YGRKKRRQRRR-(gly)q (SEQ ID NO: 2),
wherein the subscripts p and q are each independently an integer of from Oto 20.

35. The method according to claim 31 or 32, wherein the positively charged polylysine backbone has covalently attached thereto one or more positively charged efficiency groups having the amino acid sequence (gly)p-RKKRRQRRR-(gly)q (SEQ ID NO: 3), wherein the subscripts p and q are each independently an integer of from Oto 20.

36. The method according to any one of claims 31 to 35, wherein (i) the subscripts p and q are each independently an integer of from Oto 8; or (ii) are each independently an integer of from 2 to 5.

37. The method according to any one of claims 31 to 36, wherein the one or more positively charged efficiency groups are attached to both ends of the positively charged polylysine backbone of the positively charged carrier.

38. The method according to claim 31 or 32, wherein the positively charged carrier has the amino acid sequence RKKRRQRRRG-(K)15-GRKKRRQRRR (SEQ ID NO: 4).

39. The method according to any one of claims 31 to 38, wherein the composition does not locally diffuse from the site of injection following injection.

40. The method according to any one of claims 31 to 39, wherein the botulinum toxin is administered to the individual in an amount of about 40 U per injection treatment.

41. The method according to any one of claims 31 to 40, wherein said positively charged carrier and said botulinum toxin component are present in said pharmaceutical composition in a mass ratio of about 20,000:1 to about 55,000:1.

42. The method according to claim 41, wherein said positively charged carrier is present in said pharmaceutical composition in an amount of about 8 μg to about 15 μg per 40 U of said botulinum toxin component.

43. The method according to any one of claims 31 to 42, wherein said excipient comprises at least one component selected from the group consisting of L-Histidine, L-Histidine hydrochloride, polysorbate 20, and trehalose dihydrate.

44. The method according to claim 43, wherein said excipient comprises trehalose dihydrate.

45. The method according to any one of claims 31 to 44, wherein the duration of the treatment interval comprises greater than 32 weeks.

46. The method according to any one of claims 31 to 45, wherein the duration of the treatment interval comprises greater than 36 weeks.

47. The method according to any one of claims 31 to 46, wherein the duration of the treatment interval comprises at least 32 weeks to 40 weeks.

48. The method according to any one of claims 31 to 47, wherein the therapeutic or cosmetic effect is reduction of wrinkles, lines, or furrows of the individual.

49. The method according to claim 48, wherein the therapeutic or cosmetic effect is reduction in the severity of glabellar lines.

50. The method according to claim 48 or 49, wherein the administration comprises at least one injection into one or more muscle selected from the group consisting of the right corrugator muscle, the left corrugator muscle, and the procerus muscle.

51. The method according to claim 50, wherein about 8 U of said botulinum toxin component are injected into said the medial aspect of the right corrugator muscle, about 8 U of said botulinum toxin component are injected into said the lateral aspect of the right corrugator muscle; about 8 U of said botulinum toxin component are injected into said the medial aspect of the left corrugator muscle, about 8 U of said botulinum toxin component are injected into said the lateral aspect of the left corrugator muscle; and about 8 U of said botulinum toxin component are injected into a procerus muscle.

52. The method according to any one of claims 31 to 47, wherein the therapeutic or cosmetic effect is reduction of a symptom associated with a disorder selected from the group consisting of hemifacial spasm, adult onset spasmodic torticollis, anal fissure, blepharospasm, cerebral palsy, cervical dystonia, plantar fasciitis, migraine headaches, strabismus, temporomandibular joint disorder, neurologic pain, overactive bladder, rhinitis, sinusitis, acne, dystonia, dystonic contractions, hyperhidrosis, and hypersecretion of a gland controlled by the cholinergic nervous system.

53. The method or pharmaceutical composition for use according to any one of claims 1 to 52, wherein the therapeutic or cosmetic effect endures for at least about 4 weeks in over 55% of individuals each administered the pharmaceutical composition.

54. The method or pharmaceutical composition for use according to claim 53, wherein the therapeutic or cosmetic effect endures for at least about 4 weeks in over 60% of individuals administered the pharmaceutical compositions.

55. The method or pharmaceutical composition for use according to claim 54, wherein the therapeutic or cosmetic effect endures for at least about 4 weeks in over 70% of individuals administered the pharmaceutical compositions.

56. The method or pharmaceutical composition for use according to any one of claims 1 to 52, wherein the therapeutic or cosmetic effect endures for at least about 16 weeks in over 35% of individuals each administered the pharmaceutical composition.

57. The method or pharmaceutical composition for use according to claim 56, wherein the therapeutic or cosmetic effect endures for at least about 16 weeks in over 50% of individuals administered the pharmaceutical compositions.

58. The method or pharmaceutical composition for use according to claim 57, wherein the therapeutic or cosmetic effect endures for at least about 16 weeks in over 70% of individuals administered the pharmaceutical compositions.

59. The method or pharmaceutical composition for use according to any one of claims 1 to 52, wherein the therapeutic or cosmetic effect endures for at least about 24 weeks in over 15% of individuals each administered the pharmaceutical composition.

60. The method or pharmaceutical composition for use according to claim 59, wherein the therapeutic or cosmetic effect endures for at least about 24 weeks in over 25% of individuals administered the pharmaceutical compositions.

61. A method of reducing a wrinkle, line, or furrow in an individual in need thereof, the method comprising:
administering to the individual by injection to one or more muscles or facial structures associated with the wrinkle, line, or furrow of the individual a composition comprising:
a pharmaceutically acceptable diluent for injection;
a botulinum toxin component that is botulinum toxin of serotype A having a molecular weight of 150 kDa without accessory non-toxin proteins;
a positively charged carrier having the amino acid sequence RKKRRQRRRG-(K)15-GRKKRRQRRR (SEQ ID NO: 4);
wherein the botulinum toxin component is administered to the individual m a treatment dose amount of about 40 U per injection treatment;
wherein the positively charged carrier is non-covalently associated with the botulinum component; and
wherein the injection of the composition provides a single treatment dose having at least about a 26-week duration of effect in reducing the wrinkle, line, or furrow of the individual, thereby extending treatment interval duration for the individual.

62. A pharmaceutical composition in a sterile injectable formulation for use in reducing a wrinkle, line, or furrow in an individual in need thereof, said composition comprising:
a botulinum toxin component in a dose of about 40 U, that is botulinum toxin of serotype A having a molecular weight of 150 kDa without accessory proteins,
a positively charged carrier having the amino acid sequence RKKRRQRRRG-(K)15-GRKKRRQRRR (SEQ ID NO: 4); and
a pharmaceutically acceptable diluent for injection;
wherein the positively charged carrier is non-covalently associated with the botulinum toxin component; and
wherein said dose of the composition provides a single treatment having at least about a 26-week duration of effect in reducing the wrinkle, line, or furrow of the individual, thereby extending treatment interval duration for the individual.

63. The method according to claim 61, or the pharmaceutical composition for use according to claim 2, wherein the composition achieves an extended duration of effect for at least about 27 weeks.

64. The method according to claim 61, or the pharmaceutical composition for use according to claim 2, wherein the composition achieves an extended duration of effect for at least about 28 weeks.

65. The method according to claim 61, or the pharmaceutical composition for use according to claim 4, wherein the composition achieves an extended duration of effect for at least about 30 weeks.

66. The method or pharmaceutical composition for use according to any one of claims 61 to 65, wherein said positively charged carrier and said botulinum toxin component are present in said pharmaceutical composition in a mass ratio of about 20,000:1 to about 55,000:1.

67. The method or pharmaceutical composition for use according to claim 66, wherein said positively charged carrier is present in said pharmaceutical composition in an amount of 8 μg to about 15 μg per 40 U of said botulinum toxin component.

68. The method or pharmaceutical composition for use according to any one of claims 61 to 67, wherein said excipient comprises at least one component selected from the group consisting of L-Histidine, L-Histidine hydrochloride, polysorbate 20, and trehalose dihydrate.

69. The method or pharmaceutical composition for use according to claim 68, wherein said excipient comprises trehalose dihydrate.

70. The method or pharmaceutical composition for use according to any one of claims 61 to 69, wherein the reduction in the wrinkle, line, or furrow comprises a reduction in the severity of a glabellar line.

71. The method or pharmaceutical composition for use according to any one of claim 61 to 70, wherein the administration comprises at least one injection into one or more muscle selected from the group consisting of the right corrugator muscle, the left corrugator muscle, and the procerus muscle.

72. The method or pharmaceutical composition for use according to claim 71, wherein about 8 U of said botulinum toxin component are injected into the medial aspect of the right corrugator muscle, about 8 U of said botulinum toxin component are injected into the lateral aspect of the right corrugator muscle; about 8 U of said botulinum toxin component are injected into the medial aspect of the left corrugator muscle, about 8 U of said botulinum toxin component are injected into the lateral aspect of the left corrugator muscle;
and about 8 U of said botulinum toxin component are injected into a procerus muscle.

73. The method or pharmaceutical composition for use according to any one of claims 61 to 72, wherein the reduction in the wrinkle, line, or furrow endures for at least about 4 weeks in over 55% of individuals each administered the pharmaceutical composition.

74. The method or pharmaceutical composition for use according to claim 73, wherein the reduction in the wrinkle, line, or furrow endures for at least about 4 weeks in over 60% of individuals each administered the pharmaceutical composition.

75. The method or pharmaceutical composition for use according to claim 74, wherein the reduction in the wrinkle, line, or furrow endures for at least about 4 weeks in over 70% of individuals each administered the pharmaceutical composition.

76. The method or pharmaceutical composition for use according to any one of claims 61 to 72, wherein the reduction in the wrinkle, line, or furrow endures for at least about 16 weeks in over 35% of individuals each administered the pharmaceutical composition.

77. The method or pharmaceutical composition for use according to claim 76, wherein the reduction in the wrinkle, line, or furrow endures for at least about 16 weeks in over 50% of individuals each administered the pharmaceutical composition.

78. The method or pharmaceutical composition for use according to claim 77, wherein the reduction in the wrinkle, line, or furrow endures for at least about 16 weeks in over 70% of individuals each administered the pharmaceutical composition.

79. The method or pharmaceutical composition for use according to any one of claims 61 to 72, wherein the reduction in the wrinkle, line, or furrow endures for at least about 24 weeks in over 15% of individuals each administered the pharmaceutical composition.

80. The method or pharmaceutical composition for use according to claim 79, wherein the reduction in the wrinkle, line, or furrow endures for at least about 24 weeks in over 25% of individuals each administered the pharmaceutical composition.

81. A method of treating a glabellar line an individual in need thereof with injectable botulinum toxin, wherein the method comprises a treatment course having multiple treatment intervals with prolonged duration of effect and duration of treatment intervals, the treatment course comprising:
administering by injection an initial treatment dose of a sterile injectable composition into the glabellar complex of the individual to achieve a reduction in the severity of the glabellar line following the initial treatment with the composition;
wherein the composition comprises a pharmaceutically acceptable diluent suitable for injection;
a botulinum toxin component that is botulinum toxin of serotype A having a molecular weight of 150 kDa without accessory proteins; and
a positively charged carrier having the amino acid sequence RKKRRQRRRG-(K)15-GRKKRRQRRR (SEQ ID NO: 4);
wherein the botulinum toxin component is administered to the individual in a treatment dose of about 40 U per injection treatment;
wherein the positively charged carrier is non-covalently associated with the botulinum toxin component;
wherein the initial treatment dose of the composition administered by injection to the individual provides a duration of effect lasting through at least about 26 weeks; and
administering subsequent treatment doses of the composition by injection to the individual at treatment intervals comprising a duration of greater than or equal to about 26 weeks to at least about 40 weeks following the initial treatment dose and between each subsequent treatment dose.

82. The method according to claim 81, wherein the composition achieves an extended duration of effect for at least about 27 weeks.

83. The method according to claim 81, wherein the composition achieves an extended duration of effect for at least about 28 weeks.

84. The method according to claim 81, wherein the composition achieves an extended duration of effect for at least about 29 weeks.

85. The method according to claim 81, wherein the duration of the treatment interval comprises at least 30 weeks.

86. The method according to any one of claims 81 to 85, wherein said positively charged carrier and said botulinum toxin component are present in said pharmaceutical composition in a mass ratio of about 20,000:1 to about 55,000:1.

87. The method according to claim 86, wherein said positively charged carrier is present in said pharmaceutical composition in an amount of about 8 μg to about 15 μg per 40 U of said botulinum toxin component.

88. The method according to any one of claims 81 to 87, wherein said excipient comprises at least one component selected from the group consisting of L-Histidine, L-Histidine hydrochloride, polysorbate 20, and trehalose dihydrate.

89. The method according to claim 88, wherein said excipient comprises trehalose dihydrate.

90. The method according to any one of claims 81 to 89, wherein the reduction comprises a reduction in the severity of the glabellar line as assessed by Investigator Global Assessment-Facial Wrinkle Severity (IGA-FWS) and/or Patient Facial Wrinkle Severity (PFWS.)

91. The method according to any one of claims 81 to 90, wherein the administration comprises at least one injection into one or more muscle selected from the group consisting of the right corrugator muscle, the left corrugator muscle, and the procerus muscle.

92. The method according to claim 91, wherein about 8 U of said botulinum toxin component are injected into the medial aspect of the right corrugator muscle, about 8 U of said botulinum toxin component are injected into the lateral aspect of the right corrugator muscle; about 8 U of said botulinum toxin component are injected into the medial aspect of the left corrugator muscle, about 8 U of said botulinum toxin component are injected into the lateral aspect of the left corrugator muscle; and about 8 U of said botulinum toxin component are injected into a procerus muscle.

93. The method according to any one of claims 81 to 92, wherein the reduction in the glabellar line endures for at least about 4 weeks in over 55% of individuals each administered the pharmaceutical composition.

94. The method according to claim 93, wherein the reduction in the glabellar line endures for at least about 4 weeks in over 60% of individuals each administered the pharmaceutical composition.

95. The method according to claim 94, wherein the reduction in the glabellar line endures for at least about 4 weeks in over 70% of individuals each administered the pharmaceutical composition.

96. The method according to any one of claims 81 to 92, wherein the reduction in the glabellar line endures for at least about 16 weeks in over 35% of individuals each administered the pharmaceutical composition.

97. The method according to claim 96, wherein the reduction in the glabellar line endures for at least about 16 weeks in over 50% of individuals each administered the pharmaceutical composition.

98. The method according to claim 97, wherein the reduction in the glabellar line endures for at least about 16 weeks in over 70% of individuals each administered the pharmaceutical composition.

99. The method according to any one of claims 81 to 92, wherein the reduction in the glabellar line endures for at least about 24 weeks in over 15% of individuals each administered the pharmaceutical composition.

100. The method according to claim 99, wherein the reduction in the glabellar line endures for at least about 24 weeks in over 25% of individuals each administered the pharmaceutical composition.

101. A method of increasing botulinum toxin duration of action for reducing wrinkles, lines, or furrows in an individual in need thereof, said method comprising administering a plurality of successive botulinum toxin treatments,
wherein a first treatment of a botulinum toxin composition is administered to the individual by injection to or near a wrinkle, line, or furrow, said composition comprising:
a pharmaceutically acceptable diluent for injection;
a botulinum toxin component in a dose of about 10 U to about 100 U, said botulinum toxin component comprising botulinum toxin of serotype A having a molecular weight of 150 kDa without accessory proteins, and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20;
wherein the positively charged earner IS non-covalently associated with the botulinum component; and
wherein at least one successive treatment repeating said administering step is administered to said individual;
wherein said first treatment reduces said wrinkle, line, or furrow for at least about 20 weeks, and wherein said one or more successive treatments reduces said wrinkle, line, or furrow for a longer duration than achieved following said first treatment.

102. A pharmaceutical composition in sterile injectable formulations for use in increasing botulinum toxin duration of action for reducing wrinkles, lines, or furrows in an individual in need thereof, for repeat administration in a plurality of successive treatments, said composition comprising:
a pharmaceutically acceptable diluent for injection;
a botulinum toxin component in a dose of about 10 U to about 100 U, said botulinum toxin component comprising botulinum toxin of serotype A having a molecular weight of 150 kDa without accessory proteins, and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20;
wherein the positively charged earner is non-covalently associated with the botulinum toxin component;
wherein at least one successive treatment is administered to the individual; and
wherein said first treatment reduces said wrinkle, line, or furrow for at least about 20 weeks, and wherein said one or more successive treatments reduces said wrinkle, line, or furrow for a longer duration than achieved following said first treatment.

103. A method of increasing likelihood of achieving a botulinum toxin response of reducing a wrinkle, line, or furrow in an individual in need thereof, said method comprising administering a plurality of successive botulinum toxin treatments,
wherein a first treatment of a botulinum toxin composition is administered to an individual by injection to or near a wrinkle, line, or furrow, said composition comprising:
a pharmaceutically acceptable diluent suitable for injection;
a botulinum toxin component in a dose of about 10 U to about 100 U, said botulinum toxin component comprising botulinum toxin of serotype A having a molecular weight of 150 kDa without accessory proteins; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20;
wherein the positively charged earner IS non-covalently associated with the botulinum toxin component; and
wherein at least one successive treatment repeating said administering step is administered to said individual;
wherein said one or more successive treatments has a greater likelihood of reducing the wrinkle, line, or furrow in said individual compared with said first treatment.

104. A pharmaceutical composition in sterile injectable formulations for use in increasing likelihood of reducing a wrinkle, line, or furrow in an individual in need thereof, for repeat administration in a plurality of successive treatments, said composition comprising:
a pharmaceutically acceptable diluent suitable for injection;
a botulinum toxin component in a dose of about 10 U to about 100 U, said botulinum toxin component comprising botulinum toxin of serotype A having a molecular weight of 150 kDa without accessory proteins; and
a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3),
wherein the subscripts p and q are each independently an integer of from 0 to 20;
wherein the positively charged earner is non-covalently associated with the botulinum toxin component;
wherein at least one successive treatment is administered to said individual; and
wherein said one or more successive treatments has a greater likelihood of reducing the wrinkle, line, or furrow in said individual compared with said first treatment.

105. The method according to claim 101 or 103, or the pharmaceutical composition for use according to claim 102 or 104, wherein the positively charged polylysine backbone has covalently attached thereto one or more positively charged efficiency groups having the amino acid sequence (gly)p-RGRDDRRQRRR-(gly)q (SEQ ID NO: 1), wherein the subscripts p and q are each independently an integer of from Oto 20.

106. The method according to claim 101 or 103, or the pharmaceutical composition for use according to claim 102 or 104, wherein the positively charged polylysine backbone has covalently attached thereto one or more positively charged efficiency groups having the amino acid sequence (gly)p-YGRKKRRQRRR-(gly)q (SEQ ID NO: 2), wherein the subscripts p and q are each independently an integer of from Oto 20.

107. The method according to claim 101 or 103, or the pharmaceutical composition for use according to claim 102 or 104, wherein the positively charged polylysine backbone has covalently attached thereto one or more positively charged efficiency groups having the amino acid sequence (gly)p-RKKRRQRRR-(gly)q (SEQ ID NO: 3), wherein the subscripts p and q are each independently an integer of from Oto 20.

108. The method or pharmaceutical composition for use according to any one of claims 101 to 107, wherein (i) the subscripts p and q are each independently an integer of from Oto 8; or (ii) are each independently an integer of from 2 to 5.

109. The method or pharmaceutical composition for use according to any one of claims 101 to 108, wherein the one or more positively charged efficiency groups are attached to both ends of the positively charged polylysine backbone of the positively charged carrier.

110. The method or pharmaceutical composition for use according to claim 109, wherein the positively charged carrier has the amino acid sequence RKKRRQRRRG-(K)15-GRKKRRQRRR (SEQ ID NO: 4).

111. The method or pharmaceutical composition for use according to claim 110, wherein the composition achieves an extended duration of action for at least about 24 weeks following said first treatment.

112. The method or pharmaceutical composition for use according to claim 110, wherein the composition achieves an extended duration of action for at least about 25 weeks following said first treatment.

113. The method or pharmaceutical composition for use according to claim 110, wherein the composition achieves an extended duration of action for at least about 26 weeks following said first treatment.

114. The method or pharmaceutical composition for use according to claim 110, wherein the composition achieves an extended duration of action for at least about 28 weeks following said first treatment.

115. The method or pharmaceutical composition for use according to claim 110, wherein the composition achieves an extended duration of action for at least about 30 weeks following said first treatment.

116. The method or pharmaceutical composition for use according to claim 110, wherein the composition achieves an extended duration of action for at least about 32 weeks following said first treatment.

117. The method or pharmaceutical composition for use according to claim 110, wherein the composition achieves an extended duration of action for at least about 34 weeks following said first treatment.

118. The method or pharmaceutical composition for use according to claim 110, wherein the composition achieves an extended duration of action for at least about 35 weeks following said first treatment.

119. The method or pharmaceutical composition for use according to claim 110, wherein the composition achieves an extended duration of action for at least about 36 weeks following said first treatment.

120. The method or pharmaceutical composition for use according to any one of claims 110-119, wherein at least one said successive treatment is administered about 12 weeks to about 36 weeks following a prior treatment.

121. The method or pharmaceutical composition for use according to any one of claims 110-119, wherein at least one said successive treatment is administered about 16 weeks to about 32 weeks following a prior treatment.

122. The method or pharmaceutical composition for use according to any one of claims 110-119, wherein at least one said successive treatment is administered about 20 weeks to about 36 weeks following a prior treatment.

123. The method or pharmaceutical composition for use according to any one of claims 110-119, wherein at least one said successive treatment is administered about 22 weeks to about 34 weeks following a prior treatment.

124. The method or pharmaceutical composition for use according to any one of claims 110-119, wherein at least one said successive treatment is administered about 24 weeks to about 32 weeks following a prior treatment.

125. The method or pharmaceutical composition for use according to any one of claims 110-119, wherein at least one said successive treatment is administered about 26 weeks to about 30 weeks following a prior treatment.

126. The method or pharmaceutical composition for use according to any one of claims 120-125, wherein said successive treatment is the second treatment.

127. The method or pharmaceutical composition for use according to any one of claims 110-126, wherein the treatment dose of botulinum toxin administered to the individual is about 20 U to about 60 U per injection treatment.

128. The method or pharmaceutical composition for use according claim 127, wherein the treatment dose of botulinum toxin administered to the individual is about 40 U per injection treatment.

129. The method or pharmaceutical composition for use according to any one of claims 101-128, wherein said positively charged carrier and said botulinum toxin component are present in said pharmaceutical composition in a mass ratio of about 20,000:1 to about 55,000:1.

130. The method or pharmaceutical composition for use according to claim 129, wherein said positively charged carrier is present in said pharmaceutical composition in an amount of about 8 μg to about 15 μg per 40 U of said botulinum toxin component.

131. The method or pharmaceutical composition for use according to claim 130, wherein said positively charged carrier is present in said pharmaceutical composition in an amount of 11.7 μg per 40 U.

132. The method or pharmaceutical composition for use according to any one of claims 101-131, wherein said excipient comprises at least one component selected from the group consisting of L-Histidine, L-Histidine hydrochloride, polysorbate 20, and trehalose dihydrate.

133. The method or pharmaceutical composition for use according to claim 132, wherein said excipient comprises trehalose dihydrate.

134. The method or pharmaceutical composition for use according to any one of claims 101-133, wherein the wrinkle, line, or furor is a glabellar line.

135. The method or pharmaceutical composition for use according to claim 134, wherein the administration comprises at least one injection into one or more muscle selected from the group

136. The method or pharmaceutical composition for use according to claim 135, or according to any one of claims 100-109, wherein about 16 U of said botulinum toxin component are injected into said right corrugator muscle, about 16 U of said botulinum toxin component are injected into said left corrugator muscle, and about 8 U of said botulinum toxin component are injected into a procerus muscle.

137. The method or pharmaceutical composition for use according to claim 136, wherein about 8 U of said botulinum toxin component are injected into said the medial aspect of the right corrugator muscle, about 8 U of said botulinum toxin component are injected into said the lateral aspect of the right corrugator muscle; about 8 U of said botulinum toxin component are injected into said the medial aspect of the left corrugator muscle, about 8 U of said botulinum toxin component are injected into said the lateral aspect of the left corrugator muscle; and about 8 U of said botulinum toxin component are injected into a procerus muscle.

138. The method or pharmaceutical composition for use according to any one of claims 101-137, wherein side effects of botulinum toxin administration are reduced following administration of successive treatments after the first treatment.

139. The method or pharmaceutical composition for use according to claim 138, wherein the reduction of side effects is a reduction in eyelid ptosis.

140. The method or pharmaceutical composition for use according to any one of claims 101-139, wherein length of an interval between said subsequent botulinum toxin treatments is greater than the interval between the first and second treatments.

141. The method or pharmaceutical composition for use according claim 140, wherein the interval between subsequent botulinum toxin treatments is greater than about 20 weeks to about 36 weeks.

142. The method or pharmaceutical composition for use according claim 140, wherein the interval between subsequent botulinum toxin treatments is greater than about 22 weeks to about 34 weeks.

143. The method or pharmaceutical composition for use according claim 140, wherein the interval between subsequent botulinum toxin treatments is greater than about 24 weeks to about 32 weeks.

144. The method or pharmaceutical composition for use according claim 140, wherein the interval between subsequent botulinum toxin treatments is greater than about 26 weeks to about 30 weeks.

145. The method or pharmaceutical composition for use according to any one of claims 101-144, wherein the reduction in the wrinkle, line, or furrow endures for at least about 4 weeks in at least 80% of individuals following administration of the first treatment dose, and/or for at least 85% of individuals following administration of the second or subsequent treatment dose.

146. The method or pharmaceutical composition for use according to any one of claims 101-144, wherein the reduction in the wrinkle, line, or furrow endures for at least about 4 weeks in at least 85% of individuals following administration of the first treatment dose, and/or for at least 90% of individuals following administration of the second or subsequent treatment dose.

147. The method or pharmaceutical composition for use according to any one of claims 101-144, wherein the reduction in the wrinkle, line, or furrow endures for at least about 4 weeks in at least 90% of individuals following administration of the first treatment dose, and/or for at least 95% of individuals following administration of the second or subsequent treatment dose.

148. The method or pharmaceutical composition for use according to any one of claims 101-147, wherein said reduction in the wrinkle, line, or furrow corresponds to a score of 0 or 1 on IGA-FWS.

149. The method or pharmaceutical composition for use according to any one of claims 101-147, wherein said reduction in the wrinkle, line, or furrow corresponds to a score of 0 or 1 on PFWS.

150. The method or pharmaceutical composition for use according to any one of claims 17, 42, 67, or 87, wherein said positively charged carrier is present in said pharmaceutical composition in an amount of 11.7 μg per 40 U.

151. The method or pharmaceutical composition for use according to any one of the above where the subject is at least 65 years of age.
